(19) United States
(12) Patent Application Publication
(10) Pub. No.: US 2014/0348880 A1
(43) Pub. Date: Nov. 27, 2014

(54) Ortho-Aminothiophenol Compounds and Uses Thereof

(71) Applicant: Cape Breton University, Sydney (CA)

(72) Inventors: Matthias Bierenstiel, Sydney (CA); Harshiny Muthukumar, Muthukumar (IN)

(21) Appl. No.: 14/286,376

(22) Filed: May 23, 2014

Related U.S. Application Data
(60) Provisional application No. 61/827,376, filed on May 24, 2013.

(51) Int. Cl. C07C 323/36; A61L 31/16; C07D 401/12; A01N 55/02; C07D 409/12; A01N 43/10; C07D 213/53; C07F 1/08; A01N 33/00; A01N 43/40

(52) U.S. Cl. CPC C07C 323/36; A01N 33/00; A61L 31/16; C07D 401/12; A01N 43/40; C07D 409/12; A01N 43/10; C07D 213/53; C07F 1/08; A01N 55/02; C07C 2101/16
USPC 424/400; 514/646; 564/440; 514/332; 546/264; 514/444; 549/59; 514/357; 546/334; 514/184; 549/3; 546/12; 514/499; 556/34

(57) ABSTRACT

A method for treating or preventing a microbial infection in a subject in need thereof, the method comprising administering to the subject, a therapeutically effective amount of a compound of Formula Ia, a compound of Formula IIa, one or more compounds of Formula Ia or Formula IIa complexed with a metal core, or a pharmaceutically acceptable salt, prodrug or hydrate thereof.

### CROSS-REFERENCE TO RELATED APPLICATIONS

This application claims the benefit of U.S. Provisional Application No. 61/827,326, filed on May 24, 2013, the content of which is incorporated by reference herein in its entirety.

### BACKGROUND

The statements in this section merely provide background information related to the present disclosure and may not constitute prior art.

The development of new resistant strains of bacteria to current antibiotics poses a serious threat to public health. There is resurgence in research and development efforts to develop new classes of antimicrobials. Many pathogens have been repeatedly exposed to commonly-used antibiotics. This exposure has led to the selection of variant antibacterial strains resistant to a broad spectrum of antibiotics. The loss of potency and effectiveness of an antibiotic caused by resistant mechanisms renders the antibiotic ineffective and consequently can lead to life-threatening infections that are virtually untreatable. Several clinically important bacterial strains are now overcoming potent antibiotics reserved as last-line defense measures. In one example, antibiotic-resistant enterococci e.g., E. faecium are particularly significant cause of bloodstream infection in hospitalized patients, endocarditis, catheter-associated bacteremia, meningitis, and intra-abdominal infection. Those susceptible to infection include patients with neutropenia and/or cancer, patients receiving long-term hemodialysis, and liver transplant recipients.

In another clinically relevant example, Pseudomonas aeruginosa is an invasive, gram-negative bacterial pathogen that causes a wide range of severe infections which may cause morbidity in immunocompromised subjects e.g., caused by HIV infection, chemotherapy, or immunosuppressive therapy. Furthermore, P. aeruginosa causes serious infections of the lower respiratory tract, the urinary tract, and wounds in younger and older hospitalized ill patients, including those suffering from cystic fibrosis. As with Acinetobacter species and ESBL-producing Enterobacteriaceae, the incidence of P. aeruginosa infection among intensive care unit patients is increasing. Moreover, P. aeruginosa is much more difficult to treat due to its inherent ability to resist antibiotic treatment than most gram-positive and many gram-negative pathogens. New synthetic antimicrobial agents can lead to treatment options against not only “natural” pathogens, but also intermediate drug resistant and drug resistant pathogens.

What are needed are effective antimicrobial compounds that offer enhanced antimicrobial activity concomitant with reduced toxicity and side effects.

### SUMMARY

The following only summarizes certain aspects of the invention and is not intended to be limiting in nature. These aspects and other aspects and embodiments are described more fully below. All references cited in this specification are hereby incorporated by reference in their entirety. In the event of a discrepancy between the express disclosure of this specification and the references incorporated by reference, the express disclosure of this specification shall control.

It can be an object of the present invention to provide such compounds having antimicrobial activity, particularly, antibacterial activity against both Gram-positive and Gram-negative bacteria, with lower mammalian cytotoxicities and negligible hemolysis, at such concentrations, as compared to compounds of the prior art.

In a first aspect, the present invention provides a method for the treatment or prevention of a microbial infection in a subject in need thereof. The method comprises administering to the subject a compound of Formula I, a compound of Formula II, a compound of Formula I or a compound of Formula II complexed with a metal core, or a pharmaceutically acceptable salt, prodrug or hydrate thereof wherein,

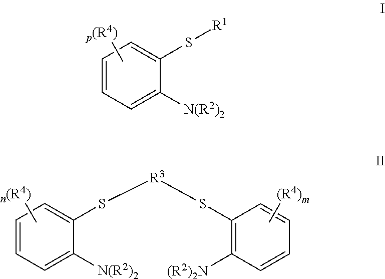

wherein,

Each R¹ is C₁₋₆ alkyl, C₂₋₆ alkenyl, aryl, heteroaryl, C₃₋₁₀ cycloaliphatic, or 5-10 membered heterocycloaliphatic having 1-3 heteroatoms independently selected from N, O, or S, any of which is optionally substituted;

Each R² is —Z^{A}R⁵, wherein each Z^{A} is independently a bond or an optionally substituted branched or straight C₁₋₆ aliphatic chain wherein up to two carbon units of Z^{A} are optionally and independently replaced by —CO—, —CS—, —CONR^{A}—, —CO₂—, —OCO—, —NR^{A}CO₂—, —O—, —NR^{A}CONR^{A}—, —OCONR^{A}—, —NR^{A}NR^{A}—, —NR^{A}CO—, —S—, —SO—, —SO₂—, —NR^{A}—, —SO₂NR^{A}—, —NR^{B}SO₂—, or —NR^{A}SO₂NR^{A}—,

Each R⁵ is independently R^{A}, halo, —OH, —NH₂, —NO₂, —CN, —CF₃, or —OCF₃,

Each R^{A} is independently hydrogen, an optionally substituted aliphatic, an optionally substituted cycloaliphatic, an optionally substituted heterocycloaliphatic, an optionally substituted aryl, or an optionally substituted heteroaryl; or

two R² groups taken together with the nitrogen atom to which they are attached form an optionally substituted 5-7 membered heterocycle having up to 3 heteroatoms, wherein up to 2 heteroatoms are independently selected from N, O, or S; or

two R² groups taken together with the nitrogen atom to which they are attached form —N═CR¹⁰R¹¹;

Each R⁴ is —Z^{B}R⁶, wherein each Z^{B} is independently a bond or an optionally substituted branched or straight C₁₋₆ aliphatic chain wherein up to two carbon units of Z^{B} are optionally and independently replaced by —CO—, —CS—, —CONR^{B}—, —CO₂—, —OCO—, —NR^{B}CO₂—, —O—, —NR^{B}CONR^{B}—, —OCONR^{B}—, —NR^{B}NR^{B}—, —NR^{B}CO—, —S—, —SO—, —SO₂—, —NR^{B}—, —SO₂NR^{B}—, —NR^{B}SO₂—, or —NR^{B}SO₂NR^{B}—,

Each R⁶ is independently R^{B}, halo, —OH, —NH₂, —NO₂, —CN, —CF₃, or —OCF₃,

Each R^{B} is independently hydrogen, an optionally substituted aliphatic, an optionally substituted cycloaliphatic, an optionally substituted heterocycloaliphatic, an optionally substituted aryl, or an optionally substituted heteroaryl; or

two R⁴ groups together with the carbon atoms to which they are attached form an optionally substituted 5-6 membered ring having 0-3 heteroatoms independently selected from N, O, or S;

Each of R¹⁰ and R¹¹ is independently is —Z^{C}R⁷, wherein each Z^{C} is independently a bond or an optionally substituted branched or straight C₁₋₆ aliphatic chain wherein up to two carbon units of Z^{C} are optionally and independently replaced by —CO—, —CS—, —CONR^{C}—, —CO₂—, —OCO—, —NR^{C}CO₂—, —O—, —NR^{C}CONR^{C}—, —OCONR^{C}—, —NR^{C}NR^{C}—, —NR^{C}CO—, —S—, —SO—, —SO₂—, —NR^{C}—, —SO₂NR^{C}—, —NR^{C}SO₂—, or —NR^{C}SO₂NR^{C}—,

Each R⁷ is independently R^{C}, halo, —OH, —NH₂, —NO₂, —CN, —CF₃, or —OCF₃,

Each R^{C} is independently hydrogen, an optionally substituted aliphatic, an optionally substituted cycloaliphatic, an optionally substituted heterocycloaliphatic, an optionally substituted aryl, or an optionally substituted heteroaryl;

R³ is —X^{A}—R⁷—X^{A}—, wherein each X^{A} is independently a bond or an optionally substituted C₁₋₆ alkylidene chain, R⁷ is a bond, or an optionally substituted aryl, or an optionally substituted heteroaryl; and

Each of m, n, and p is independently 0 or a positive integer from 1-3.

In a second aspect, the metal core is an alkali metal, an alkali earth metal, or a transition metal. For example, in another aspect, the metal core is Cu, Ag, or Au. In some aspects of the method, the compound is a compound of Formula I.

In a third aspect, the compound is a compound of Formula I, and R¹ is C₁₋₆ alkyl, optionally substituted with aryl or heteroaryl, C₁₋₆ alkyl optionally substituted with phenyl, nephthyl, pyridine-yl, pyrimidine-yl, or pyrazine-yl, an optionally substituted phenyl or naphthyl, or thiophenyl, pyrrole-yl, pyridine-yl, pyrimidine-yl, pyrazine-yl, quinolone-yl, or quinolizine-yl, any of which is optionally substituted. In some aspects, R¹ is optionally substituted 5-10 membered mono- or bicyclic cycloaliphatic, for example, cyclopentyl, cyclohexyl, cycloheptyl, bicyclo[2.2.1]heptyl, bicyclo[2.2.2]octyl, or bicyclo[2.1.1]hexyl, any of which is optionally substituted, or R¹ is optionally substituted 5-10 membered mono- or bicyclic heterocycloaliphatic, for example, tetrahydrofuran, pyrrolidine-yl, piperidine-yl, or piperazine-yl.

In a fourth aspect, the invention provides a method for treating or preventing an infection caused by a microorganism, the method comprising: administering to a subject in need thereof, a therapeutically effective amount of a compound of Formula I or Formula II complexed with a metal core, or a pharmaceutically acceptable salt, solvate or prodrug thereof, or a pharmaceutical composition comprising a therapeutically effective amount of a compound of Formula I, a compound of Formula II, a compound of Formula I or a compound of Formula II complexed with a metal core, or a pharmaceutically acceptable salt, prodrug or hydrate thereof, and a pharmaceutically acceptable carrier, excipient, or diluent.

In a fifth aspect, the present invention provides a method for sterilizing a solid surface, the method comprising: contacting the surface, with a therapeutically effective amount of a compound of Formula I, a compound of Formula II, a compound of Formula I or a compound of Formula II complexed with a metal core, or a pharmaceutically acceptable salt, prodrug or hydrate thereof, or a pharmaceutical composition comprising a therapeutically effective amount of a compound of Formula I, a compound of Formula II, a compound of Formula I or a compound of Formula II complexed with a metal core, or a pharmaceutically acceptable salt, prodrug or hydrate thereof, and a pharmaceutically acceptable carrier, excipient, or diluent.

In a sixth aspect, the compound of Formula I, a compound of Formula II, a compound of Formula I or a compound of Formula II complexed with a metal core, or a pharmaceutically acceptable salt, prodrug or hydrate thereof may be used to coat a medical device used for diagnostic or for therapeutic treatment concomitant to other surgical procedures to prevent hospital borne nosocomial infection, the method comprising: contacting a medical device, or medical instrument of portion thereof, with a therapeutically effective amount of a compound of Formula I, a compound of Formula II, a compound of Formula I or a compound of Formula II complexed with a metal core, or a pharmaceutically acceptable salt, prodrug or hydrate thereof for a period of time sufficient to sterilize or reduce the amount of pathological microorganisms colonized on at least a portion of the medical device, or medical instrument.

In a seventh aspect, a pharmaceutical composition for use in the treatment of a microbial infection in a subject in need thereof, the pharmaceutical composition comprising a therapeutically effective amount of a compound of Formula I, a compound of Formula II, a compound of Formula I or a compound of Formula II complexed with a metal core, or a pharmaceutically acceptable salt, prodrug or hydrate thereof, or an antimicrobial composition comprising a therapeutically effective amount of a compound of Formula I, a compound of Formula II, a compound of Formula I or a compound of Formula II complexed with a metal core, or a pharmaceutically acceptable salt, prodrug or hydrate thereof, and a solvent or diluent

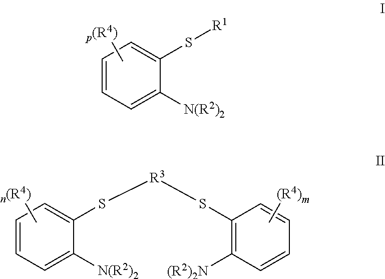

wherein,

Each R¹ is C₁₋₆ alkyl, C₂₋₆ alkenyl, aryl, heteroaryl, C₃₋₁₀ cycloaliphatic, or 5-10 membered heterocycloaliphatic having 1-3 heteroatoms independently selected from N, O, or S, any of which is optionally substituted;

Each R² is —Z^{A}R⁵, wherein each Z^{A} is independently a bond or an optionally substituted branched or straight C₁₋₆ aliphatic chain wherein up to two carbon units of Z^{A} are optionally and independently replaced by —CO—, —CS—, —CONR^{A}—, —CO₂—, —OCO—, —NR^{A}CO₂—, —O—, —NR^{A}CONR^{A}—, —OCONR^{A}—, —NR^{A}NR^{A}—, —NR^{A}CO—, —S—, —SO—, —SO₂—, —NR^{A}—, —SO₂NR^{A}—, —NR^{B}SO₂—, or —NR^{A}SO₂NR^{A}—,

Each R⁵ is independently R^{A}, halo, —OH, —NH₂, —NO₂, —CN, —CF₃, or —OCF₃,

Each R^{A} is independently hydrogen, an optionally substituted aliphatic, an optionally substituted cycloaliphatic, an optionally substituted heterocycloaliphatic, an optionally substituted aryl, or an optionally substituted heteroaryl; or

two R² groups taken together with the nitrogen atom to which they are attached form an optionally substituted 5-7 membered heterocycle having up to 3 heteroatoms, wherein up to 2 heteroatoms are independently selected from N, O, or S; or

two R² groups taken together with the nitrogen atom to which they are attached form —N═CR¹⁰R¹¹;

Each R⁴ is —Z^{B}R⁶, wherein each Z^{B} is independently a bond or an optionally substituted branched or straight C₁₋₆ aliphatic chain wherein up to two carbon units of Z^{B} are optionally and independently replaced by —CO—, —CS—, —CONR^{B}—, —CO₂—, —OCO—, —NR^{B}CO₂—, —O—, —NR^{B}CONR^{B}—, —OCONR^{B}—, —NR^{B}NR^{B}—, —NR^{B}CO—, —S—, —SO—, —SO₂—, —NR^{B}—, —SO₂NR^{B}—, —NR^{B}SO₂—, or —NR^{B}SO₂NR^{B}—,

Each R⁶ is independently R^{B}, halo, —OH, —NH₂, —NO₂, —CN, —CF₃, or —OCF₃,

Each R^{B} is independently hydrogen, an optionally substituted aliphatic, an optionally substituted cycloaliphatic, an optionally substituted heterocycloaliphatic, an optionally substituted aryl, or an optionally substituted heteroaryl; or

two R⁴ groups together with the carbon atoms to which they are attached form an optionally substituted 5-6 membered ring having 0-3 heteroatoms independently selected from N, O, or S;

Each of R¹⁰ and R¹¹ is independently is —Z^{C}R⁷, wherein each Z^{C} is independently a bond or an optionally substituted branched or straight C₁₋₆ aliphatic chain wherein up to two carbon units of Z^{C} are optionally and independently replaced by —CO—, —CS—, —CONR^{C}—, —CO₂—, —OCO—, —NR^{C}CO₂—, —O—, —NR^{C}CONR^{C}—, —OCONR^{C}—, —NR^{C}NR^{C}—, —NR^{C}CO—, —S—, —SO—, —SO₂—, —NR^{C}—, —SO₂NR^{C}—, —NR^{C}SO₂—, or —NR^{C}SO₂NR^{C}—,

Each R⁷ is independently R^{C}, halo, —OH, —NH₂, —NO₂, —CN, —CF₃, or —OCF₃,

Each R^{C} is independently hydrogen, an optionally substituted aliphatic, an optionally substituted cycloaliphatic, an optionally substituted heterocycloaliphatic, an optionally substituted aryl, or an optionally substituted heteroaryl;

R³ is —X^{A}—R⁷—X^{A}—, wherein each X^{A} is independently a bond or an optionally substituted C₁₋₆ alkylidene chain, R⁷ is a bond, or an optionally substituted aryl, or an optionally substituted heteroaryl; and

Each of m, n, and p is independently 0 or a positive integer from 1-3, wherein administration of said pharmaceutical composition to said subject provides for a minimum period of time of 6-12 hours a concentration in a target tissue of at least about 2-10 times the MIC of the bacteria.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1: ¹H NMR spectrum of 2,2′-((1,2-phenylenebis(methylene))bis(sulfanediyl))dianiline and CuCl and CuBr in CDCl₃.

FIG. 2: ¹H NMR spectrum of 2,2′-((1,2-phenylenebis(methylene))bis(sulfanediyl))dianiline and Cu (CO₂CH₃)₂, CuCl₂ and Cu(ClO₄)₂

FIG. 3: ¹H NMR spectrum of 2,2′-((1,2-phenylenebis(methylene))bis(sulfanediyl))dianiline+CuBr and CuCl

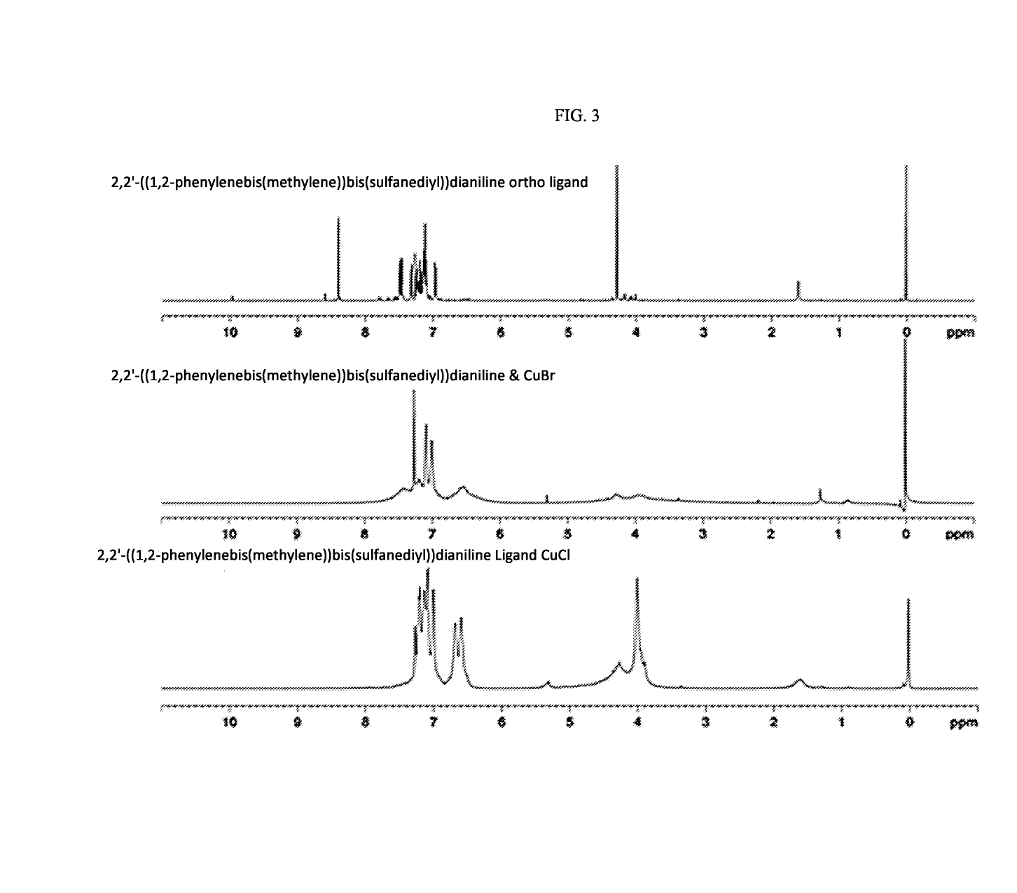

FIG. 4: Mass Spectrum (MS) of 2,2′-((1,2-phenylenebis(methylene))bis(sulfanediyl))dianiline in acetonitrile.

FIG. 5: Mass Spectrum (MS) of 2,2′-((1,2-phenylenebis(methylene))bis(sulfanediyl))dianiline+Cu(OAc)₂ in MeOH.

FIG. 6: Mass Spectrum (MS) of 2,2′-((1,2-phenylenebis(methylene))bis(sulfanediyl))dianiline+CuCl₂.

### DETAILED DESCRIPTION

The following description is merely exemplary in nature and is not intended to limit the present disclosure, application, or uses.

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

The headings (such as “Introduction” and “Summary”) and sub-headings used herein are intended only for general organization of topics within the present technology, and are not intended to limit the disclosure of the present technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. Any discussion of the content of references cited in the Introduction is intended merely to provide a general summary of assertions made by the authors of the references, and does not constitute an admission as to the accuracy of the content of such references. All references cited in the “Description” section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

Although the open-ended term “comprising,” as a synonym of terms such as including, containing, or having, is use herein to describe and claim the present invention, the invention, or embodiments thereof, may alternatively be described using more limiting terms such as “consisting of” or “consisting essentially of” the recited ingredients.

### DEFINITIONS

The symbol “—” means a single bond, “═” means a double bond, “≡” means a triple bond, “ [symbol] ” means a single or double bond. The symbol “ [symbol] ” refers to a group on a double-bond as occupying either position on the terminus of a double bond to which the symbol is attached; that is, the geometry, E- or Z-, of the double bond is ambiguous. When a group is depicted removed from its parent Formula, the “ [symbol] ” symbol will be used at the end of the bond which was theoretically cleaved in order to separate the group from its parent structural Formula.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual Formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

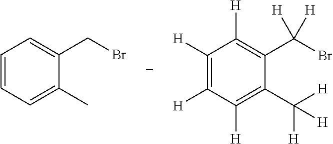

If a group “R” is depicted as “floating” on a ring system, as for example in the Formula:

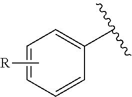

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused or bridged ring system, as for example:

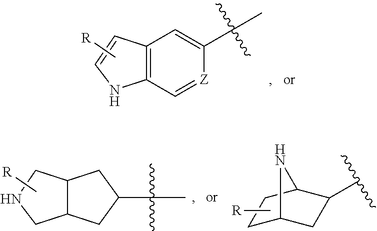

then, unless otherwise defined, a substituent “R” may reside on any atom of the fused or bridged ring system, assuming replacement of a depicted hydrogen (for example the —NH— in the Formula above), implied hydrogen (for example as in the Formula above, where the hydrogens are not shown but understood to be present), or expressly defined hydrogen (for example where in the Formula above, “Z” equals ═CH—) from one of the ring atoms, so long as a stable structure is formed. In the example depicted, the “R” group may reside on either the 5-membered or the 6-membered ring of the fused or bridged ring system.

When a group “R” is depicted as existing on a ring system containing saturated carbons, as for example in the Formula:

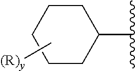

where, in this example, “y” can be more than one, assuming each replaces a currently depicted, implied, or expressly defined hydrogen on the ring; then, unless otherwise defined, where the resulting structure is stable, two “R's” may reside on the same carbon. In another example, two R's on the same carbon, including that carbon, may form a ring, thus creating a spirocyclic ring structure with the depicted ring as for example in the Formula:

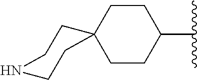 [SMILES: *C1CCC2(CCNCC2)CC1]

“Acyl” means a —C(O)R radical where R is alkyl, haloalkyl, alkenyl, cycloalkyl, cycloalkylalkyl, aryl, aralkyl, heteroaryl, heteroaralkyl, heterocycloalkyl, or heterocycloalkylalkyl, as defined herein, e.g., acetyl, trifluoromethylcarbonyl, or 2-methoxyethylcarbonyl, and the like.

“Acylamino” means a —NRR′ radical where R is hydrogen, hydroxy, alkyl, or alkoxy and R′ is acyl, as defined herein.

“Acyloxy” means an —OR radical where R is acyl, as defined herein, e.g. cyanomethylcarbonyloxy, and the like.

“Administration” and variants thereof (e.g., “administering” a compound) in reference to a compound of the invention means introducing the compound of the invention into the system of the animal, for example, a human in need of treatment. When a compound of the invention or prodrug thereof is provided in combination with one or more other active agents (e.g., surgery, radiation, and chemotherapy, etc.), “administration” and its variants are each understood to include concurrent and sequential introduction of the compound or prodrug thereof and other agents.

“Alkenyl” means a means a linear monovalent hydrocarbon radical of two to six carbon atoms or a branched monovalent hydrocarbon radical of three to 6 carbon atoms which radical contains at least one double bond, e.g., ethenyl, propenyl, 1-but-3-enyl, and 1-pent-3-enyl, and the like.

“Alkoxy” means an —OR group where R is alkyl group as defined herein. Examples include methoxy, ethoxy, propoxy, isopropoxy, and the like.

“Alkoxyalkyl” means an alkyl group, as defined herein, substituted with at least one, specifically one, two, or three, alkoxy groups as defined herein. Representative examples include methoxymethyl and the like.

“Alkoxycarbonyl” means a —C(O)R group where R is alkoxy, as defined herein.

“Alkyl” means a linear saturated monovalent hydrocarbon radical of one to six carbon atoms or a branched saturated monovalent hydrocarbon radical of three to 6 carbon atoms, e.g., methyl, ethyl, propyl, 2-propyl, butyl (including all isomeric forms), or pentyl (including all isomeric forms), and the like.

“Alkylamino” means an —NHR group where R is alkyl, as defined herein.

“Alkylaminoalkyl” means an alkyl group substituted with one or two alkylamino groups, as defined herein.

“Alkylaminoalkyloxy” means an —OR group where R is alkylaminoalkyl, as defined herein.

“Alkylcarbonyl” means a —C(O)R group where R is alkyl, as defined herein.

“Alkylsulfonyl” means an —S(O)₂R group where R is alkyl, as defined herein.

“Alkylsulfonylalkyl” means an alkyl group, as defined herein, substituted with at least one, preferably one or two, alkylsulfonyl groups, as defined herein.

“Alkynyl” means a linear monovalent hydrocarbon radical of two to six carbon atoms or a branched monovalent hydrocarbon radical of three to 6 carbon atoms which radical contains at least one triple bond, e.g., ethynyl, propynyl, butynyl, pentyn-2-yl and the like.

“Amino” means —NH₂.

“Aminoalkyl” means an alkyl group substituted with at least one, specifically one, two or three, amino groups.

“Aminoalkyloxy” means an —OR group where R is aminoalkyl, as defined herein.

“Aminocarbonyl” means a —C(O)NH₂ group.

“Alkylaminocarbonyl” means a —C(O)NHR group where R is alkyl as defined herein.

“Aryl” means a monovalent six- to fourteen-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical, valency rules permitting. Representative examples include phenyl, naphthyl, and indanyl, and the like.

“Arylalkyl” means an alkyl radical, as defined herein, substituted with one or two aryl groups, as defined herein, e.g., benzyl and phenethyl, and the like.

“Arylalkyloxy” means an —OR group where R is arylakyl, as defined herein.

“Cyanoalkyl” means an alkyl group, as defined herein, substituted with one or two cyano groups.

“Cycloalkyl” means a monocyclic or fused or bridged bicyclic or tricyclic, saturated or partially unsaturated (but not aromatic), monovalent hydrocarbon radical of three to ten carbon ring atoms. Unless stated otherwise, the valency of the group may be located on any atom of any ring within the radical, valency rules permitting. One or two ring carbon atoms may be replaced by a —C(O)—, —C(S)—, or —C(═NH)— group. More specifically, the term cycloalkyl includes, but is not limited to, cyclopropyl, cyclobutyl, cyclopentyl, cyclohexyl, cyclohexyl, cyclohex-3-enyl, or (1r,3r,5R,7R)-tricyclo[3.3.1.1^{3,7}]decan-2-yl, and the like.

“Cycloalkylalkyl” means an alkyl group substituted with at least one, specifically one or two, cycloalkyl group(s) as defined herein.

“Dialkylamino” means a —NRR′ radical where R and R′ are alkyl as defined herein, or an N-oxide derivative, or a protected derivative thereof, e.g., dimethylamino, diethylamino, N,N-methylpropylamino or N,N-methylethylamino, and the like.

“Dialkylaminoalkyl” means an alkyl group substituted with one or two dialkylamino groups, as defined herein.

“Dialkylaminoalkyloxy” means an —OR group where R is dialkylaminoalkyl, as defined herein. Representative examples include 2-(N,N-diethylamino)-ethyloxy, and the like.

“Dialkylaminocarbonyl” means a —C(O)NRR′ group where R and R′ are alkyl as defined herein.

“Halogen” or “halo” refers to fluorine, chlorine, bromine and iodine.

“Haloalkoxy” means an —OR′ group where R′ is haloalkyl as defined herein, e.g., trifluoromethoxy or 2,2,2-trifluoroethoxy, and the like.

“Haloalkyl” mean an alkyl group substituted with one or more halogens, specifically 1, 2, 3, 4, 5, or 6 halo atoms, e.g., trifluoromethyl, 2-chloroethyl, and 2,2-difluoroethyl, and the like.

“Heteroaryl” means a monocyclic or fused or bridged bicyclic monovalent radical of 5 to 14 ring atoms containing one or more, specifically one, two, three, or four ring heteroatoms where each heteroatom is independently —O—, —S(O)ₙ— (n is 0, 1, or 2), —NH—, —N═, or N-oxide, with the remaining ring atoms being carbon, wherein the ring comprising a monocyclic radical is aromatic and wherein at least one of the fused rings comprising the bicyclic radical is aromatic. One or two ring carbon atoms of any nonaromatic rings comprising a bicyclic radical may be replaced by a —C(O)—, —C(S)—, or —C(═NH)— group. Unless stated otherwise, the valency may be located on any atom of any ring of the heteroaryl group, valency rules permitting. More specifically, the term heteroaryl includes, but is not limited to, 1,2,4-triazolyl, 1,3,5-triazolyl, phthalimidyl, pyridinyl, pyrrolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, and the like), isoindolyl, indolinyl, isoindolinyl, benzimidazolyl, benzodioxol-4-yl, benzofuranyl, cinnolinyl, indolizinyl, naphthyridin-3-yl, phthalazin-3-yl, phthalazin-4-yl, pteridinyl, purinyl, quinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, 2,3-dihydrobenzofuranyl, benzo[d][1,3]dioxolyl, 2,3-dihydrobenzo[b][1,4]dioxinyl, thiazolyl, isothiazolyl, thiadiazolyl, benzothiazolyl, benzothienyl, and the derivatives thereof, or N-oxide or a protected derivative thereof. The term “5- or 6-membered heteroaryl” describes a subset of the term “heteroaryl.”

“Heteroarylalkyl” means an alkyl group, as defined herein, substituted with at least one, specifically one or two heteroaryl group(s), as defined herein.

“Heterocycloalkyl” means a saturated or partially unsaturated (but not aromatic) monovalent monocyclic group of 3 to 8 ring atoms or a saturated or partially unsaturated (but not aromatic) monovalent fused or bridged, bicyclic or tricyclic group of 5 to 12 ring atoms in which one or more, specifically one, two, three, or four ring heteroatoms where each heteroatom is independently O, S(O)ₙ (n is 0, 1, or 2), —N═, or —NH—, the remaining ring atoms being carbon. One or two ring carbon atoms may be replaced by a —C(O)—, —C(S)—, or —C(═NH)— group. Unless otherwise stated, the valency of the group may be located on any atom of any ring within the radical, valency rules permitting. When the point of valency is located on a nitrogen atom, R^{y} is absent. More specifically the term heterocycloalkyl includes, but is not limited to, azetidinyl, pyrrolidinyl, 2-oxopyrrolidinyl, 2,5-dihydro-1H-pyrrolyl, piperidinyl, 4-piperidonyl, morpholinyl, piperazinyl, 2-oxopiperazinyl, tetrahydropyranyl, 2-oxopiperidinyl, thiomorpholinyl, thiamorpholinyl, perhydroazepinyl, pyrazolidinyl, imidazolinyl, imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydrocyclopenta[c]pyrrolyl, octahydroindolyl, octahydroisoindolyl, decahydroisoquinolyl, tetrahydrofuryl, tetrahydropyranyl, (3aR,6aS)-5-methyloctahydrocyclopenta[c]pyrrolyl, and (3aS,6aR)-5-methyl-1,2,3,3a,4,6a-hexahydrocyclopenta[c]pyrrolyl, and the derivatives thereof and N-oxide or a protected derivative thereof.

“Heterocycloalkylalkyl” means an alkyl radical, as defined herein, substituted with one or two heterocycloalkyl groups, as defined herein, e.g., morpholinylmethyl, N-pyrrolidinylethyl, and 3-(N-azetidinyl)propyl, and the like.

“Heterocycloalkyloxy” means an —OR group where R is heterocycloalkyl, as defined herein.

“Hydroxyalkyl” means an alkyl group, as defined herein, substituted with at least one, preferably 1, 2, 3, or 4, hydroxy groups.

“Phenylalkyl” means an alkyl group, as defined herein, substituted with one or two phenyl groups.

“Phenylalkyloxy” means an —OR group where R is phenylalkyl, as defined herein.

“Optional” or “optionally” means that the subsequently described event or circumstance may or may not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term, unless stated otherwise. A list of exemplary optional substitutions is presented below in the definition of “substituted.”

“Optionally substituted aryl” means an aryl group, as defined herein, optionally substituted with one, two, or three substituents independently acyl, acylamino, acyloxy, alkyl, haloalkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, or aminoalkoxy; or aryl is pentafluorophenyl. Within the optional substituents on “aryl”, the alkyl and alkenyl, either alone or as part of another group (including, for example, the alkyl in alkoxycarbonyl), are independently optionally substituted with one, two, three, four, or five halo.

“Optionally substituted arylalkyl” means an alkyl group, as defined herein, substituted with optionally substituted aryl, as defined herein.

“Optionally substituted cycloalkyl” means a cycloalkyl group, as defined herein, substituted with one, two, or three groups independently acyl, acyloxy, acylamino, alkyl, haloalkyl, alkenyl, alkoxy, alkenyloxy, alkoxycarbonyl, alkenyloxycarbonyl, alkylthio, alkylsulfinyl, alkylsulfonyl, aminosulfonyl, alkylaminosulfonyl, dialkylaminosulfonyl, alkylsulfonylamino, halo, hydroxy, amino, alkylamino, dialkylamino, aminocarbonyl, alkylaminocarbonyl, dialkylaminocarbonyl, nitro, alkoxyalkyloxy, aminoalkoxy, alkylaminoalkoxy, dialkylaminoalkoxy, carboxy, or cyano. Within the above optional substituents on “cycloalkyl”, the alkyl and alkenyl, either alone or as part of another substituent on the cycloalkyl ring, are independently optionally substituted with one, two, three, four, or five halo, e.g. haloalkyl, haloalkoxy, haloalkenyloxy, or haloalkylsulfonyl.

“Optionally substituted cycloalkylalkyl” means an alkyl group substituted with at least one, specifically one or two, optionally substituted cycloalkyl groups, as defined herein.

“Optionally substituted heteroaryl” means a heteroaryl group optionally substituted with one, two, or three substituents independently acyl, acylamino, acyloxy, alkyl, haloalkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, alkylaminoalkoxy, or dialkylaminoalkoxy. Within the optional substituents on “heteroaryl”, the alkyl and alkenyl, either alone or as part of another group (including, for example, the alkyl in alkoxycarbonyl), are independently optionally substituted with one, two, three, four, or five halo.

“Optionally substituted heteroarylalkyl” means an alkyl group, as defined herein, substituted with at least one, specifically one or two, optionally substituted heteroaryl group(s), as defined herein.

“Optionally substituted heterocycloalkyl” means a heterocycloalkyl group, as defined herein, optionally substituted with one, two, or three substituents independently acyl, acylamino, acyloxy, haloalkyl, alkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, or phenylalkyl. Within the optional substituents on “heterocycloalkyl”, the alkyl and alkenyl, either alone or as part of another group (including, for example, the alkyl in alkoxycarbonyl), are independently optionally substituted with one, two, three, four, or five halo.

“Optionally substituted heterocycloalkylalkyl” means an alkyl group, as defined herein, substituted with at least one, specifically one or two, optionally substituted heterocycloalkyl group(s) as defined herein.

“Optionally substituted phenyl” means a phenyl group optionally substituted with one, two, or three substituents independently acyl, acylamino, acyloxy, alkyl, haloalkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, or aminoalkoxy, or aryl is pentafluorophenyl. Within the optional substituents on “phenyl”, the alkyl and alkenyl, either alone or as part of another group (including, for example, the alkyl in alkoxycarbonyl), are independently optionally substituted with one, two, three, four, or five halo.

“Optionally substituted phenylalkyl” means an alkyl group, as defined herein, substituted with one or two optionally substituted phenyl groups, as defined herein.

“Optionally substituted phenylsulfonyl” means an —S(O)₂R group where R is optionally substituted phenyl, as defined herein.

“Oxo” means an oxygen which is attached via a double bond.

“Yield” for each of the reactions described herein is expressed as a percentage of the theoretical yield.

“Metabolite” refers to the break-down or end product of a compound or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 8.sup.th Ed., Pergamon Press, Gilman et al. (eds), 1990 for a discussion of biotransformation). As used herein, the metabolite of a compound of the invention or its salt may be the biologically active form of the compound in the body. In one example, a prodrug may be used such that the biologically active form, a metabolite, is released in vivo. In another example, a biologically active metabolite is discovered serendipitously, that is, no prodrug design per se was undertaken. An assay for activity of a metabolite of a compound of the present invention is known to one of skill in the art in light of the present disclosure.

“Prodrug” refers to compounds that are transformed (typically rapidly) in vivo to yield the parent compound of the invention, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Patient” and “Subject” are used interchangeably herein and for the purposes of the present invention include humans and other animals, particularly mammals, and other organisms, plants and cell cultures. Thus the methods are applicable to both human therapy and veterinary applications. In a specific embodiment the subject is a mammal, and in a more specific embodiment the subject is a human.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Specific salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine.

“Therapeutically effective amount” is an amount of a compound of the invention, that when administered to a patient, prevents, inhibits or ameliorates a symptom of the microbial infection. The term “effective concentration” or “effective amount” means that a sufficient amount of the antimicrobial compound is added to decrease, prevent or inhibit the growth of microbial organisms, for example, bacterial organisms or bacterial colonization. The term “inhibiting” or “reducing” as used herein, is taken to mean the act of limiting the growth of microbes or pathogenic bacteria. The amount will vary for each compound and upon known factors such as pharmaceutical characteristics; the type of medical device; age, sex, health and weight of the recipient subject; and the use and length of use. It is within one of ordinary skill in the art's ability to relatively easily determine an effective concentration for each compound provided herein. The therapeutically effective amount can be determined routinely by one of ordinary skill in the art having regard to their knowledge in the antimicrobial arts, routine titration practices, performance in clinical trials using subjects of various sex, age and disease conditions, and to this disclosure without under experimentation.

“Preventing” or “prevention” of a microbial infection, includes inhibiting the microbial infection from occurring in a subject, e.g. a human, i.e. causing the clinical symptoms of the infection not to develop in a subject that may be exposed to or predisposed to the infection but does not yet experience or display symptoms of the infection.

“Treating” or “treatment” of a disease, disorder, or syndrome, as used herein, include alleviating, abating or ameliorating a microbial infection, symptoms, preventing additional symptoms, inhibiting the infection, e.g., arresting the development of the infection, relieving the infection, causing regression of the infection, relieving a condition caused by the infection, or stopping the symptoms of the infection either prophylactically and/or therapeutically. In some embodiments, the microbial infection is a bacterial infection, a viral infection, a fungal infection or a protozoan infection. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art. Treatment as a prophylactic measure is also included. Treatment includes combination treatments and therapies, in which two or more treatments or therapies are combined, for example, sequentially or simultaneously.

“Co-administration” or “combined administration” or the like as utilized herein are meant to include modes of administration of the selected active, therapeutic agents to a single patient, and are intended to include treatment regimens in which the agents are not necessarily administered by the same route of administration or at the same time. Co-administration can also include delivery of the active ingredients in a “fixed combination,” e.g. a compound of Formulae I, or II as the first active agent, and an antibiotic, an antiviral agent, an antifungal agent or an antiprotozoan agent as a second active agent, which are both administered to a patient simultaneously in the form of a single entity or dosage. The term “non-fixed combination” means that the active ingredients, e.g. a compound of Formula I, Formula II, Formula Ia, Formula Ib, Formula IIa, metal complexes thereof, or pharmaceutically acceptable salts, prodrugs and hydrates thereof and a second active agent, for example, an antibiotic agent as exemplified below, are both administered to a patient as separate entities either simultaneously, concurrently or sequentially with no specific time limits, such that the administration provides therapeutically effective levels of the combination of active agents in the body of the patient.

### A. Compounds

The present invention relates to compounds useful as antimicrobial agents for the treatment and prevention of microbial infection. In some embodiments, compounds of the present invention, including methods of synthesizing said compounds are described in Cross, E. D., et. al., “Synthesis and Characterization of Donor-Functionalized N,S-Compounds Containing the ortho-Aminothiophenol Motif”, Synthesis, (2011) No. 2, pp 303-315, the disclosure of which is incorporated herein by reference in its entirety.

In some embodiments, compounds of the present invention include a compound of Formula I, a compound of Formula II, a compound of Formula I or a compound of Formula II complexed with a metal core, or a pharmaceutically acceptable salt, prodrug or hydrate thereof,

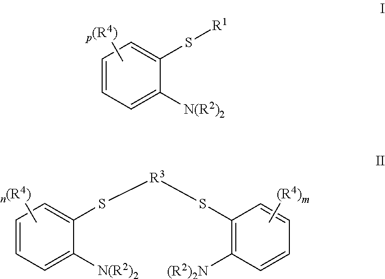

wherein,

Each R¹ is C₁₋₆ alkyl, C₂₋₆ alkenyl, aryl, heteroaryl, C₃₋₁₀ cycloaliphatic, or 5-10 membered heterocycloaliphatic having 1-3 heteroatoms independently selected from N, O, or S, any of which is optionally substituted;

Each R² is —Z^{A}R⁵, wherein each Z^{A} is independently a bond or an optionally substituted branched or straight C₁₋₆ aliphatic chain wherein up to two carbon units of Z^{A} are optionally and independently replaced by —CO—, —CS—, —CONR^{A}—, —CO₂—, —OCO—, —NR^{A}CO₂—, —O—, —NR^{A}CONR^{A}—, —OCONR^{A}—, —NR^{A}NR^{A}—, —NR^{A}CO—, —S—, —SO—, —SO₂—, —NR^{A}—, —SO₂NR^{A}—, —NR^{B}SO₂—, or —NR^{A}SO₂NR^{A}—,

Each R⁵ is independently R^{A}, halo, —OH, —NH₂, —NO₂, —CN, —CF₃, or —OCF₃,

Each R^{A} is independently hydrogen, an optionally substituted aliphatic, an optionally substituted cycloaliphatic, an optionally substituted heterocycloaliphatic, an optionally substituted aryl, or an optionally substituted heteroaryl; or

two R² groups taken together with the nitrogen atom to which they are attached form an optionally substituted 5-7 membered heterocycle having up to 3 heteroatoms, wherein up to 2 heteroatoms are independently selected from N, O, or S; or

two R² groups taken together with the nitrogen atom to which they are attached form —N═CR¹⁰R¹¹;

Each R⁴ is —Z^{B}R⁶, wherein each Z^{B} is independently a bond or an optionally substituted branched or straight C₁₋₆ aliphatic chain wherein up to two carbon units of Z^{B} are optionally and independently replaced by —CO—, —CS—, —CONR^{B}—, —CO₂—, —OCO—, —NR^{B}CO₂—, —O—, —NR^{B}CONR^{B}—, —OCONR^{B}—, —NR^{B}NR^{B}—, —NR^{B}CO—, —S—, —SO—, —SO₂—, —NR^{B}—, —SO₂NR^{B}—, —NR^{B}SO₂—, or —NR^{B}SO₂NR^{B}—,

Each R⁶ is independently R^{B}, halo, —OH, —NH₂, —NO₂, —CN, —CF₃, or —OCF₃,

Each R^{B} is independently hydrogen, an optionally substituted aliphatic, an optionally substituted cycloaliphatic, an optionally substituted heterocycloaliphatic, an optionally substituted aryl, or an optionally substituted heteroaryl; or

two R⁴ groups together with the carbon atoms to which they are attached form an optionally substituted 5-6 membered ring having 0-3 heteroatoms independently selected from N, O, or S;

Each of R¹⁰ and R¹¹ is independently is —Z^{C}R⁷, wherein each Z^{C} is independently a bond or an optionally substituted branched or straight C₁₋₆ aliphatic chain wherein up to two carbon units of Z^{C} are optionally and independently replaced by —CO—, —CS—, —CONR^{C}—, —CO₂—, —OCO—, —NR^{C}CO₂—, —O—, —NR^{C}CONR^{C}—, —OCONR^{C}—, —NR^{C}NR^{C}—, —NR^{C}CO—, —S—, —SO—, —SO₂—, —NR^{C}—, —SO₂NR^{C}—, —NR^{C}SO₂—, or —NR^{C}SO₂NR^{C}—,

Each R⁷ is independently R^{C}, halo, —OH, —NH₂, —NO₂, —CN, —CF₃, or —OCF₃,

Each R^{C} is independently hydrogen, an optionally substituted aliphatic, an optionally substituted cycloaliphatic, an optionally substituted heterocycloaliphatic, an optionally substituted aryl, or an optionally substituted heteroaryl;

R³ is —X^{A}—R⁷—X^{A}—, wherein each X^{A} is independently a bond or an optionally substituted C₁₋₆ alkylidene chain, R⁷ is a bond, or an optionally substituted aryl, or an optionally substituted heteroaryl; and

Each of m, n, and p is independently 0 or a positive integer from 1-3.

In some embodiments, the metal core is an alkali metal, an alkali earth metal, or a transition metal, for example, the metal core is Cu, Ag, or Au.

In some embodiments, the compound is a compound of Formula I, and R¹ is C₁₋₆ alkyl, optionally substituted with aryl or heteroaryl.

In some embodiments, the compound is a compound of Formula I, and R¹ is C₁₋₆ alkyl optionally substituted with phenyl, nephthyl, pyridine-yl, pyrimidine-yl, or pyrazine-yl.

In some embodiments, the compound is a compound of Formula I, and R¹ is optionally substituted phenyl or naphthyl.

In some embodiments, the compound is a compound of Formula I, and R¹ is thiophenyl, pyrrole-yl, pyridine-yl, pyrimidine-yl, pyrazine-yl, quinolone-yl, or quinolizine-yl, any of which is optionally substituted.

In some embodiments, the compound is a compound of Formula I, and R¹ is optionally substituted 5-10 membered mono- or bicyclic cycloaliphatic.

In some embodiments, R¹ is cyclopentyl, cyclohexyl, cycloheptyl, bicyclo[2.2.1]heptyl, bicyclo[2.2.2]octyl, or bicyclo[2.1.1]hexyl, any of which is optionally substituted.

In some embodiments, the compound is a compound of Formula I, and R¹ is optionally substituted 5-10 membered mono- or bicyclic heterocycloaliphatic, for example, R¹ is tetrahydrofuran, pyrrolidine-yl, piperidine-yl, or piperazine-yl.

In some embodiments, a method for treating or preventing a microbial infection in a subject in need thereof, comprises administering to the subject, a therapeutically effective amount of a compound of Formula Ia, a compound of Formula IIa, or one or two compounds independently selected from of Formula Ia or Formula IIa complexed with a metal core, or a pharmaceutically acceptable salt, prodrug or hydrate thereof,

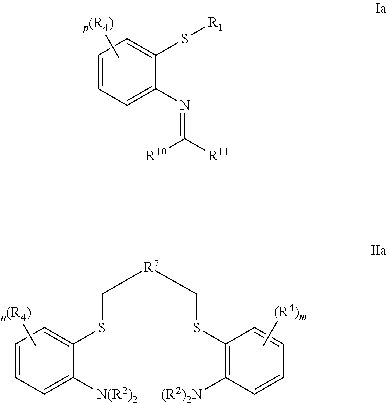

wherein,

R¹ is C₁₋₆ alkyl, C₂₋₆ alkenyl, aryl, heteroaryl, C₃₋₁₀ cycloaliphatic, or 5-10 membered heterocycloaliphatic having 1-3 heteroatoms independently selected from N, O, or S, any of which is optionally substituted;

R² is independently hydrogen, or each pair of R² groups is —N═CR¹⁰R¹¹, wherein one of R¹⁰ and R¹¹ is hydrogen and the other is optionally substituted phenyl or an optionally substituted 5-6 membered heteroaryl having 1-2 heteroatoms independently selected from N, O, or S;

R⁴ is —Z^{B}R⁶, wherein each Z^{B} is independently a bond or an optionally substituted branched or straight C₁₋₆ aliphatic chain wherein up to two carbon units of Z^{B} are optionally and independently replaced by —CO—, —CS—, —CONR^{B}—, —CO₂—, —OCO—, —NR^{B}CO₂—, —O—, —NR^{B}CONR^{B}—, —OCONR^{B}—, —NR^{B}NR^{B}—, —NR^{B}CO—, —S—, —SO—, —SO₂—, —NR^{B}—, —SO₂NR^{B}—, —NR^{B}SO₂—, or —NR^{B}SO₂NR^{B}—;

R⁶ is independently R^{B}, halo, —OH, —NH₂, —NO₂, —CN, —CF₃, or —OCF₃, R^{B} is independently hydrogen, an optionally substituted aliphatic, an optionally substituted cycloaliphatic, an optionally substituted heterocycloaliphatic, an optionally substituted aryl, or an optionally substituted heteroaryl; or

two R⁴ groups together with the carbon atoms to which they are attached form an optionally substituted 5-6 membered ring having 0-3 heteroatoms independently selected from N, O, or S;

R⁷ is an optionally substituted phenyl or an optionally substituted 6 membered heteroaryl having 1-2 heteroatoms independently selected from N, O, or S;

One of R¹⁰ and R¹¹ is hydrogen and the other is optionally substituted phenyl or an optionally substituted 5-6 membered heteroaryl having 1-2 heteroatoms independently selected from N, O, and S; and

Each of m, n, and p is independently 0 or a positive integer from 1-3.

In some embodiments, the compound is a compound of Formula I, and the compound of Formula I is selected from a compound of Formula Ib

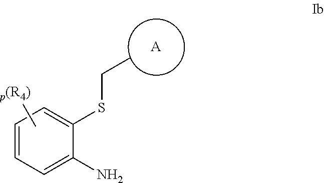

wherein

Each R⁴ is —Z^{B}R⁶, wherein each Z^{B} is independently a bond or an optionally substituted branched or straight C₁₋₆ aliphatic chain wherein up to two carbon units of Z^{B} are optionally and independently replaced by —CO—, —CS—, —CONR^{B}—, —CO₂—, —OCO—, —NR^{B}CO₂—, —O—, —NR^{B}CONR^{B}—, —OCONR^{B}—, —NR^{B}NR^{B}—, —NR^{B}CO—, —S—, —SO—, —SO₂—, —NR^{B}—, —SO₂NR^{B}—, —NR^{B}SO₂—, or —NR^{B}SO₂NR^{B}—,

Each R⁶ is independently R^{B}, halo, —OH, —NH₂, —NO₂, —CN, —CF₃, or —OCF₃,

Each R^{B} is independently hydrogen, an optionally substituted aliphatic, an optionally substituted cycloaliphatic, an optionally substituted heterocycloaliphatic, an optionally substituted aryl, or an optionally substituted heteroaryl; or

two R⁴ groups together with the carbon atoms to which they are attached form an optionally substituted 5-6 membered ring having 0-3 heteroatoms independently selected from N, O, or S; and

Ring A is optionally substituted phenyl or an optionally substituted 6 membered heteroaryl.

In some embodiments, the compound of Formulas Ia, and Ib, or one or more compounds selected independently from Formula Ia and/or Formula Ib are complexed with a metal core, or a pharmaceutically acceptable salt, prodrug or hydrate thereof.

In some embodiments of the compounds of Formula Ia, R¹ is benzyl optionally substituted at the phenyl ring; and

one of R¹⁰ and R¹¹ is hydrogen and the other is optionally substituted phenyl or an optionally substituted 5-6 membered heteroaryl having 1-2 heteroatoms independently selected from N, O, or S;

In some embodiments, the compound is a compound of Formula IIa, or a pharmaceutically acceptable salt thereof, wherein

R⁷ is an optionally substituted phenyl or an optionally substituted 6 membered heteroaryl having 1-2 heteroatoms independently selected from N, O, or S;

Each R² is hydrogen, or each pair of R² groups is —N═CR¹⁰R¹¹, wherein one of R¹⁰ and R¹¹ is hydrogen and the other is optionally substituted phenyl or an optionally substituted 5-6 membered heteroaryl having 1-2 heteroatoms independently selected from N, O, or S.

In some embodiments of the compounds of Formula Ia, and IIa, or a pharmaceutically acceptable salt thereof, or one or more compounds of Formulas Ia and/or IIa each complexed with a metal core, or a pharmaceutically acceptable salt thereof, R¹ is benzyl optionally substituted at the phenyl ring, R⁴ is independently H, R² is independently H, and R¹⁰ is independently H, and R¹¹ is independently pyridyl, phenyl or thiophene. In some embodiments, R¹¹ is 2-pyridyl, phenyl, or 2-thiophene.

In some embodiments of the compounds of Formula IIa, or one or more compounds of Formula IIa complexed with a metal core, or a pharmaceutically acceptable salt thereof, R₇ is selected from ortho-xylylene and para-xylylene.

In some embodiments, one or more compounds (for example, one or two compounds) independently selected from Formula I, Formula II, Formula Ia, Formula Ib, Formula IIa, or pharmaceutically acceptable salts thereof is complexed with a metal core, for example, the metal core is Au, Ag, Cu(I) or Cu(II). In some embodiments the compound of Formula I, Formula II, Formula Ia, Formula Ib, Formula IIa, Formula Ia, Formula Ib, Formula IIa or a pharmaceutically acceptable salt thereof is complexed with a bi-metal core comprising two metal ions, for example, the bi-metal core is one or two Cu(I) ions or one or two Cu(II) ions or one Cu(I) ion and one Cu(II) ion.

In some embodiments, the compound of Formula Ia, or Formula IIa or a pharmaceutically acceptable salts thereof is complexed with a metal core, for example, the metal core is a mono Cu(I) or Cu(II) complex, wherein R¹¹ is 2-pyridyl, phenyl, or 2-thiophenyl. In some embodiments, the compound of Formula Ia, or Formula IIa or a pharmaceutically acceptable salt thereof, the compound of Formulas Ia and/or IIa, or a pharmaceutically acceptable salt thereof is complexed with a metal core, for example, the metal core is Cu(I) or Cu(II), wherein R¹¹ is 2-pyridyl. In some embodiments, the compound of Formula Ia, or Formula IIa or a pharmaceutically acceptable salt thereof is complexed with a metal core, for example, the metal core is Cu(I), and wherein R¹¹ is phenyl or 2-thiophene. In some of these embodiments, two compounds of Formula Ia are complexed to one or two metal ions, selected from Ag, Au, or Cu, for example, Cu(I) and/or Cu(II) metal ions. In some embodiments, the two compounds of Formula Ia can be the same or different.

In various embodiments, the compounds of Formula Ia and Formula IIa may be complexed with one or two metal ions selected from Cu, Ag, or Au, for example, Cu(I) and/or Cu(II) metal ions. As described herein, each individual compound of Formula Ia and Formula IIa may be complexed with one or two metal ions. In other embodiments, two compounds selected from Formula Ia and/or Formula IIa may be complexed with one or two metal ions selected from Cu, Ag, or Au, for example, Cu(I) and/or Cu(II) metal ions, wherein the compounds may be the same or different. In some of these embodiments, two compounds of Formula Ia are complexed to one or two metal ions, selected from Ag, Au, or Cu, for example, Cu(I) and/or Cu(II) metal ions. In some embodiments, the two compounds of Formula Ia can be the same or different. A representative illustration of two compounds of Formula Ia complexed to one or two metal ions is shown in the structure of compound: 2-(methylthio)-N-(2-pyridylmethylene)-benzenamine copper (I) iodide

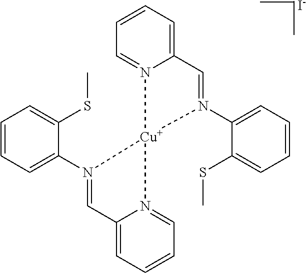

Representative compounds of Formulas I, II, Ia, Ib, IIa, pharmaceutically acceptable salts thereof and metal complexes thereof are depicted below. The examples are merely illustrative and do not limit the scope of the invention in any way. Antimicrobial compounds of the invention are named according to systematic application of the nomenclature rules agreed upon by the International Union of Pure and Applied Chemistry (IUPAC), International Union of Biochemistry and Molecular Biology (IUBMB), and the Chemical Abstracts Service (CAS). Specifically, names in Table 1 were generated using ChemDraw Ultra, Version 11.0 (3) or 12.0 (CambridgeSoft). In some embodiments, the antimicrobial compound is selected from a compound of Table 1.

TABLE 1
Exemplary compounds useful in the methods, pharmaceutical compositions and
medical devices of the present invention.

| Compound Name | Structure |
|---|---|
| 2,2′-((1,2- phenylenebis(methylene))bis(sulfanediyl))dianiline | 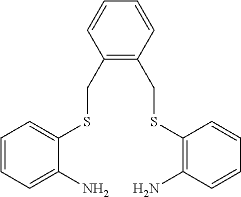 [SMILES: Nc1ccccc1SCc1ccccc1CSc1ccccc1N] |
| 2,2′-((1,4- phenylenebis(methylene))bis(sulfanediyl))dianiline. | 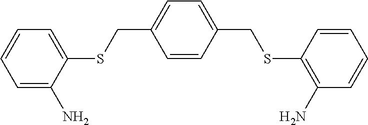 [SMILES: Nc1ccccc1SCc1ccc(CSc2ccccc2N)cc1] |
| (NZ,N′Z)-2,2′-((1,2- phenylenebis(methylene))bis(sulfanediyl))bis(N- (pyridin-2-ylmethylene)aniline) | 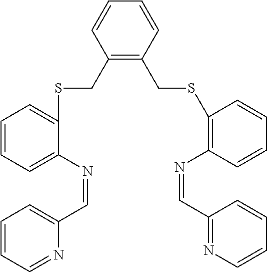 [SMILES: C(=N/c1ccccc1SCc1ccccc1CSc1ccccc1/N=C\c1ccccn1)/c1ccccn1] |
| (NZ,N′Z)-2,2′-((1,2- phenylenebis(methylene))bis(sulfanediyl))bis(N- (thiophen-2-ylmethylene)aniline) | 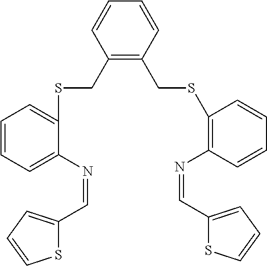 [SMILES: C(=N/c1ccccc1SCc1ccccc1CSc1ccccc1/N=C\c1cccs1)/c1cccs1] |
| (Z)-2-(benzylthio)-N-(pyridin-2-ylmethylene)aniline | 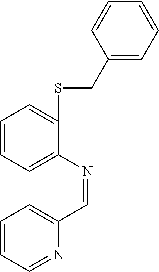 [SMILES: C(=N/c1ccccc1SCc1ccccc1)/c1ccccn1] |
| 2,2′-((1,4- phenylenebis(methylene))bis(sulfanediyl))dianiline | 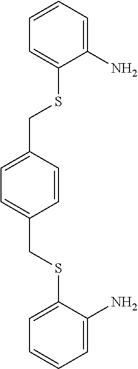 [SMILES: Nc1ccccc1SCc1ccc(CSc2ccccc2N)cc1] |
| 2,2′-((1,2- phenylenebis(methylene))bis(sulfanediyl))dianiline | 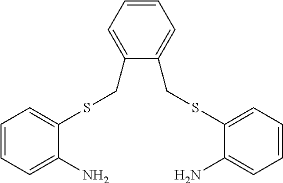 [SMILES: Nc1ccccc1SCc1ccccc1CSc1ccccc1N] |
| (E)-2-(benzylthio)-N-(pyridin-2- ylmethylene)aniline | 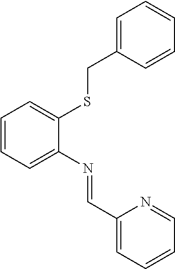 [SMILES: C(=N\c1ccccc1SCc1ccccc1)/c1ccccn1] |
| (NE,N′E)-2,2′-((1,2- phenylenebis(methylene))bis(sulfanediyl))bis(N- (pyridin-2-ylmethylene)aniline) | 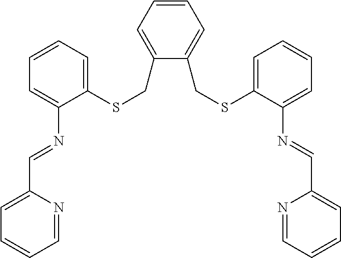 [SMILES: C(=N\c1ccccc1SCc1ccccc1CSc1ccccc1/N=C/c1ccccn1)/c1ccccn1] |
| 2-(methylthio)-N-(2-thienylmethylene)- benzenamine copper (II) bromide | 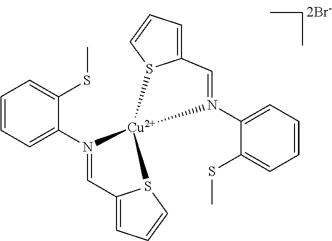 |
| 2-(methylthio)-N-(2-pyridylmethylene)- benzenamine copper (I) bromide | 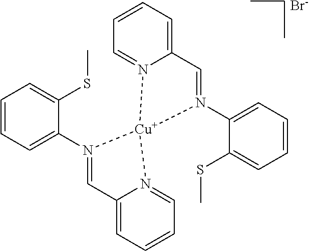 |
| 2-(methylthio)-N-(2-pyridylmethylene)- benzenamine copper (I) iodide | 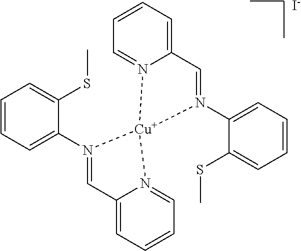 |
| 2-(methylthio)-N-(2-pyridylmethylene)- benzenamine copper (II) bromide | 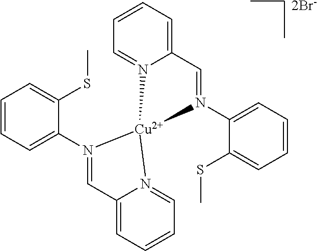 |
| 2-(methylthio)-N-(2-phenylmethylene)- benzenamine copper (II) bromide | 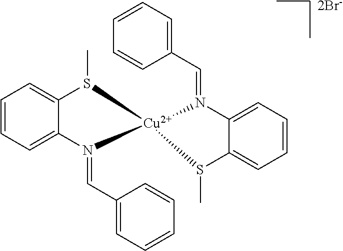 |

### B. Microbial Targets

The compounds of Formula I, Formula II, Formula Ia, Formula Ib, Formula IIa, metal complexes thereof, or pharmaceutically acceptable salts, prodrugs and hydrates thereof, are antimicrobial, i.e. the ability of a compound or combination of compounds or antimicrobial composition as described herein to beneficially control or kill pathogenic, spoilage, or otherwise harmful microorganisms, including, but not limited to, bacteria, fungi, viruses, protozoa, yeasts, mold, and mildew.

Accordingly, the compounds of Formula I, Formula II, Formula Ia, Formula Ib, Formula IIa, metal complexes thereof, or pharmaceutically acceptable salts, prodrugs and hydrates thereof of the present invention are useful, for example, for treating a localized or systemic bacterial infection in a subject, for preserving food stuff, e.g., by preventing colonization with a microorganism that causes food-poisoning in a subject or a microorganism that causes food-spoilage. For example, an antimicrobial composition of the invention is useful for preventing colonization by a bacterium, such as, for example, Staphylococcus sp., Bacillus sp., Salmonella sp., Clostridium perfringens, Campylobacter sp., Listeria monocytogenes, Vibrio parahaemolyticus, and Entero-pathogenic Escherichia coli or a fungus of the genera Aspergillus, Penicillium or Rhizopus.

The antimicrobial compounds of Formula I, Formula II, Formula Ia, Formula Ib, Formula IIa, metal complexes thereof, or pharmaceutically acceptable salts, prodrugs and hydrates thereof of the invention are useful for the treatment of an infection by a microorganism, such as, for example, a virus, a bacterium, a yeast, a fungus, or a protozoa. Organisms against which compounds of Formula I, Formula II, Formula Ia, Formula Ib, Formula IIa, metal complexes thereof, or pharmaceutically acceptable salts, prodrugs and hydrates thereof of the invention are active will be apparent to the skilled artisan and include, for example, a virus from a family selected from the group consisting of Astroviridae, Caliciviridae, Picornaviridae, Togaviridae, Flaviviridae, Caronaviridae, Paramyxviridae, Orthomyxoviridae, Bunyaviridae, Arenaviridae, Rhabdoviridae, Filoviridae, Reoviridae, Bornaviridae, Retroviridae, Poxyiridae, Herpesviridae, Adenoviridae, Papovaviridae, Parvoviridae, Hepadnaviridae, (eg., a virus selected from the group consisting of a Coxsackie A-24 virus Adenovirus 11, Adenovirus 21, Coxsackie B virus, Borna Diease Virus, Respiratory syncytial virus, Parainfluenza virus, California encephalitis virus, human papilloma virus, varicella zoster virus, Colorado tick fever virus, Herpes Simplex Virus, vaccinia virus, parainfluenza virus 1, parainfluenza virus 2, parainfluenza virus 3, dengue virus, Ebola virus, Parvovirus B19 Coxsackie A-16 virus, HSV-1, hepatitis A virus, hepatitis B virus, hepatitis C virus, hepatitis D virus, hepatitis E virus, human immunodeficiency virus, Coxsackie B1-B5, Influenza viruses A, B or C, LaCross virus, Lassayirus, rubeola virus Coxsackie A or B virus, Echovirus, lymphocytic choriomeningitis virus, HSV-2, mumps virus, Respiratory Syncytial Virus, Epstein-Barr Virus, Poliovirus Enterovirus, rabies virus, rubivirus, variola virus, WEE virus, Yellow fever virus and varicella zoster virus).

Preferably, the compounds of Formula I, Formula II, Formula Ia, Formula Ib, Formula IIa, metal complexes thereof, or pharmaceutically acceptable salts, prodrugs and hydrates thereof, are useful for the treatment and/or prevention of an infection (localized or systemic) by a bacterium, such as for example, a gram-positive bacterium or a gram-negative bacterium. For example, the present invention is useful for treating or preventing an infection caused by a bacterium, such as, for example, Streptococcus pyogenes, Streptococcus agalactiae, Streptococcus equi, Streptococcus canis, Streptococcus bovis, Streptococcus equinus, Streptococcus anginosus, Streptococcus sanguis, Streptococcus salivarius, Streptococcus mitis, Streptococcus mutans, Enterococcus faecalis, Enterococcus faecium, Staphylococcus epidermidis, Staphylococcus aureus (Staphylococcus aureus strains that are methicillin-resistant (MRSA) and vancomycin intermediate-resistant (VISA)), Aeromonas hydrophila, Bacillus cereus, Bacillus anthracis, Bacillus subtilis, Bacillus circulans, Bacillus pumilus, Bacillus licheniformis, Bacillus sphaericus, Bacillus coagulans, Hemophilus influenzae, Pseudomonas aeruginosa, Pseudomonas pseudomallei, Pseudomonas mallei, Brucella melitensis, Brucella suis, Brucella abortus, Bordetella pertussis, Neisseria meningitidis, Neisseria gonorrhoeae, Moraxella catarrhalis, Campylobacter jejuni, Clostridium difficile, Clostridium botulinum, Clostridium perfringens, Clostridium tetanii, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, Listeria monocytogenes, Nocardia asteroides, Bacteroides species, Actinomycetes species, Treponema pallidum, Leptospirosa species, Klebsiella pneumoniae; Escherichia coli, E. coli 0157:H7, Salmonella enterica, Vibrio cholerae, Vibrio parahaemolyticus, Mycobacterium tuberculosis, Proteus species, Shigella species, Serratia species, Acinetobacter, Yersinia pestis, Yersinia enterocolitica, Enterobacter species, Bacteriodes species or Legionella species. In some embodiments, the compounds of Formula Ia, or Formula IIa or a pharmaceutically acceptable salts thereof, and metal complexes thereof can be used to prevent or treat an infection in a subject caused by bacterial species Bacillus cereus and Staphylococcus aureus sp., (including Staphylococcus aureus strains that are methicillin-resistant (MRSA) and vancomycin intermediate-resistant (VISA) strains of Staphylococcus aureus).

Preferably, the antimicrobial compositions of the present invention are useful for treating and/or preventing an infection caused by a bacterium such as, for example, E. coli, B. cereus, P. vulgaris, P aeruginosa, S. aureus, S. epididermis, S. pyogenes or K. pneumonia, most preferably, B. cereus and S. aureus.

The antimicrobial compositions of the present invention is preferably also useful for treating an infection caused by a yeast or a fungus, such as, for example, Aspergillus sp., Dermatophytes, Blastomyces derinatitidis, Candida sp., (for example, Candida albicans) Malassezia furfur, Exophiala werneckii, Piedraia hortai, Trichosporon beigelii, Pseudallescheria boydii, Madurella grisea, Histoplasma capsulatum, Sporothrix schenckii, Histoplasma capsulatum, T. rubrum, T. interdigitale, T. tonsurans, M. audouini, T. violaceum, M. ferrugineum, T. schoenleinii, T. megninii, T. soudanense, T. yaoundei, M. canis, T. equinum, T. erinacei, T. verrucosum, M. distortum, M. gypseum or M. falvum.

The antimicrobial compositions of the present invention are preferably also useful for treating and/or preventing an infection caused by a protozoan organism, for example, Plasmodium malariae, P. falciparum, P. ovale, P. knowlesi, P. vivax, Leishmania tropica, Trypanosoma cruzi and T. brucei gambiense.

In some embodiments, the compounds of Formula I, Ia, Ib, II, IIa, pharmaceutically acceptable salts thereof, and metal complexes thereof can be used to disinfect, sterilize or otherwise used to kill bacteria selected from Streptococcus pyogenes, Streptococcus agalactiae, Streptococcus equi, Streptococcus canis, Streptococcus bovis, Streptococcus equinus, Streptococcus anginosus, Streptococcus sanguis, Streptococcus salivarius, Streptococcus mitis, Streptococcus mutans, Enterococcus faecalis, Enterococcus faecium, Staphylococcus epidermidis, Staphylococcus aureus (Staphylococcus aureus strains that are methicillin-resistant (MRSA) and vancomycin intermediate-resistant (VISA)), Aeromonas hydrophila, Bacillus cereus, Bacillus anthracis, Bacillus subtilis, Bacillus circulans, Bacillus pumilus, Bacillus licheniformis, Bacillus sphaericus, Bacillus coagulans, Hemophilus influenzae, Pseudomonas aeruginosa, Pseudomonas pseudomallei, Pseudomonas mallei, Brucella melitensis, Brucella suis, Brucella abortus, Bordetella pertussis, Neisseria meningitidis, Neisseria gonorrhoeae, Moraxella catarrhalis, Campylobacter jejuni, Clostridium difficile, Clostridium botulinum, Clostridium perfringens, Clostridium tetanii, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, Listeria monocytogenes, Nocardia asteroides, Bacteroides species, Actinomycetes species, Treponema pallidum, Leptospirosa species, Klebsiella pneumoniae; Escherichia coli, E. coli 0157:H7, Salmonella enterica, Vibrio cholerae, Vibrio parahaemolyticus, Mycobacterium tuberculosis, Proteus species, Shigella species, Serratia species, Acinetobacter, Yersinia pestis, Yersinia enterocolitica, Enterobacter species, Bacteriodes species or Legionella species found on a solid, flexible or porous substrate, in which bacteria are found. In some embodiments, the compounds of Formula I, Ia, Ib, II, IIa, pharmaceutically acceptable salts thereof, and metal complexes thereof can be formulated either alone or with other known bactericidal, bacteriostatic active agents discussed further herein, to form soaps, creams, detergents, solutions, and other cleansing agents known for cleaning and disinfecting human skin or surfaces that are contacted by humans, for example, eating or drinking utensils and bathroom surfaces.

### C. Pharmaceutical Compositions

a. Formulations

The antimicrobial pharmaceutical compositions and other compositions (e.g. sterilization composition, antimicrobial coating composition, preservative compositions and the like), collectively referred to as “antimicrobial compositions” unless stated otherwise, can be made using conventional procedures. For example, in some embodiments, components of the antimicrobial compositions can be conveniently dissolved or dispersed in an inert fluid medium that serves as an excipient. The term “inert” means that the excipient does not have a deleterious effect on the active ingredient(s) upon storage, nor does it substantially diminish its activity, nor does it adversely react with any other component of the composition.

In some embodiments, the invention provides pharmaceutical compositions comprising a compound of Formula I, Formula II, Formula Ia, Formula Ib, Formula IIa, metal complexes thereof, or pharmaceutically acceptable salts, prodrugs and hydrates thereof, according to the invention and a pharmaceutically acceptable carrier, excipient, or diluent. In certain embodiments, administration is by the oral route or parenterally. Administration of the compounds of the invention, or their pharmaceutically acceptable salts, prodrugs and hydrates thereof, in pure form or in an appropriate pharmaceutical composition, can be carried out via any of the accepted modes of administration or agents for serving similar utilities. Pharmaceutical preparations can be prepared in accordance with standard procedures and are administered at dosages that are selected to treat or prevent a microbial infection (see, e.g., Remington's Pharmaceutical Sciences, Mack Publishing Company, Easton, Pa. and Goodman and Gilman's “The Pharmaceutical Basis of Therapeutics,” Pergamon Press, New York, N.Y., the contents of which are incorporated herein by reference, for a general description of methods for administering various antimicrobial agents for human therapy).

Depending on the specific microorganism being treated, embodiments of the antimicrobial compositions can be formulated and administered systemically or locally. Suitable routes can include, for example, oral, rectal, transdermal, vaginal, transmucosal, or intestinal administration; parentral delivery, including, but not limited to, intravenous, intramuscular, subcutaneous, intramedullary, injections, as well as intrathecal, direct intraventricular, intraperitonial, intranesal, or intraocular injections. Dosage forms include, but are not limited to, solutions, suspensions, tablets, capsules, pills, powders, dispersions, emulsions, troches, injectable preparations, patches, ointments, creams, lotions, shampoos, dusting powders and the like.

Embodiments of pharmaceutical compositions suitable for oral administration can be presented as discrete units such as, for example, tablets, capsules, cachets, pouches, or aerosol sprays, each containing a predetermined amount of the active ingredient(s), as a powder, granules, mini or microtablets, or as a solution or a suspension in an aqueous liquid, a non-aqueous liquid, an oil-in-water emulsion, or a water-in-oil liquid emulsion. Such compositions can be prepared by any of the methods of pharmaceutical dosage preparation known in the art, but all methods include the step of bringing into association the active ingredient with the excipient, which constitutes one or more ingredients. Embodiments of the pharmaceutical compositions can be prepared by uniformly and intimately admixing the active ingredient with liquid excipients or finely divided solid excipients or both, and then, if necessary, shaping the product into the desired presentation. Methods for preparing pharmaceutical formulations are well known, methods of which are taught in Remington's Pharmaceutical Sciences. 18th Ed. Mack Printing Company, 1990, the entire disclosure of which is incorporated herein by reference in its entirety.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In some embodiments, solid oral compositions can include an amount of a compound of Formula I, Formula II, Formula Ia, Formula Ib, Formula IIa, metal complexes thereof, or pharmaceutically acceptable salts, prodrugs and hydrates thereof in an amount ranging from about 0.001 mg to about 500 mg per dose. In some embodiments, the compositions should be formulated so that a dosage of between about 0.001 to about 10,000 mg/kg body weight/day of the compound of Formula I, Formula II, Formula Ia, Formula Ib, Formula IIa, metal complexes thereof, or pharmaceutically acceptable salts, prodrugs and hydrates thereof can be administered to a patient receiving these compositions. In some embodiments, the amount of compound of Formula I, Formula II, Formula Ia, Formula Ib, Formula IIa, metal complexes thereof, or pharmaceutically acceptable salts, prodrugs and hydrates thereof administered to a subject in need thereof may be from about 0.001 mg/kg/day to about 100 mg/kg/day, from about 0.01 mg/kg/day to about 100 mg/kg/day, from about 0.1 mg/kg/day to about 100 mg/kg/day, from about 1 mg/kg/day to about 100 mg/kg/day, from about 5 mg/kg/day to about 100 mg/kg/day, or from about 10 mg/kg/day to about 100 mg/kg/day. In some embodiments, the amount of compound of Formula I, Formula II, Formula Ia, Formula Ib, Formula IIa, metal complexes thereof, or pharmaceutically acceptable salts, prodrugs and hydrates thereof administered to a subject in need thereof, may be from about 0.003 mg/kg/day to about 70 mg/kg/day. In some embodiments, amount of compound of Formula I, Formula II, Formula Ia, Formula Ib, Formula IIa, metal complexes thereof, or pharmaceutically acceptable salts, prodrugs and hydrates thereof may be from about 0.01 mg/kg/day to about 40 mg/kg/day. In some embodiment, the amount of compound of Formula I, Formula II, Formula Ia, Formula Ib, Formula IIa, metal complexes thereof, or pharmaceutically acceptable salts, prodrugs and hydrates thereof administered to a subject in need thereof may be from about 0.05 mg/kg/day to about 50 mg/kg/day. In some embodiments, the dosage may be 0.01 mg/day to 1,500 mg/day, more preferably 0.01 mg/day to 600 mg/day. The amount of compound of Formula I, Formula II, Formula Ia, Formula Ib, Formula IIa, metal complexes thereof, or pharmaceutically acceptable salts, prodrugs and hydrates thereof in the antimicrobial composition may preferably be about 0.01 mg to about 5,000 mg, about 1 mg to about 5,000 mg, from about 10 mg to about 3,000 mg, from about 50 mg to about 1,500 mg, from about 100 mg to about 1,000 mg. In some embodiments, the amount of compound of Formula I, Formula II, Formula Ia, Formula Ib, Formula IIa, metal complexes thereof, or pharmaceutically acceptable salts, prodrugs and hydrates thereof in the antimicrobial composition may be about from about 0.5 mg to about 5,000 mg, from about 1 mg to about 5,000 mg, from about 10 mg to about 5,000 mg, from about 25 mg to about 5,000 mg, from about 50 mg to about 5,000 mg, from about 100 mg to about 5,000 mg, from about 200 mg to about 5,000 mg, from 300 mg to about 5,000 mg, from about 0.5 mg to about 3,000 mg, from about 1 mg to about 3,000 mg, from about 10 mg to about 3,000 mg, from about 25 mg to about 3,000 mg, from 50 mg to about 3,000 mg, from about 0.1 mg to about 1,000 mg, from about 1 mg to about 1,000 mg, from about 10 mg to about 1,000 mg, from about 50 mg to about 1,000 mg, from about 100 mg to about 1,000 mg, from about 300 mg to about 1,000 mg, or from 450 mg to about 1,000 mg. In some embodiments, the amount of compound of Formula I, Formula II, Formula Ia, Formula Ib, Formula IIa, metal complexes thereof, or pharmaceutically acceptable salts, prodrugs and hydrates thereof is from about 15 mg to about 900 mg. These doses may be administered as a single daily dose, or may be divided into several doses administered throughout the day, for example, 1 to 5 doses, preferably two or three doses per day. In some embodiments, the amount of compound of Formula I, Formula II, Formula Ia, Formula Ib, Formula IIa, metal complexes thereof, or pharmaceutically acceptable salts, prodrugs and hydrates thereof administered as a daily dose or one or more divided doses (1-5) throughout the day, is from about 0.05 mg to about 1,000 mg. In some embodiments, the therapeutically effective amount of compound of Formula I, Formula II, Formula Ia, Formula Ib, Formula IIa, metal complexes thereof, or pharmaceutically acceptable salts, prodrugs and hydrates thereof is from about 0.5 mg to about 800 mg dosed daily. In some embodiments, the therapeutically effective amount of compound of Formula I, Formula II, Formula Ia, Formula Ib, Formula IIa, metal complexes thereof, or pharmaceutically acceptable salts, prodrugs and hydrates thereof is from about 1 mg to about 600 mg dosed daily. In some embodiments, the therapeutically effective amount of a compound of Formula I, Formula II, Formula Ia, Formula Ib, Formula IIa, metal complexes thereof, or pharmaceutically acceptable salts, prodrugs and hydrates thereof is from about 500 mg to about 1,000 mg dosed daily.

In some embodiments, the pharmaceutical composition comprises at least about 50 mg of a compound of Formula I, Formula II, Formula Ia, Formula Ib, Formula IIa, metal complexes thereof, or pharmaceutically acceptable salts, prodrugs and hydrates thereof. In some embodiments, the pharmaceutical composition comprises at least about 0.025 mg of a compound of Formula I, Formula II, Formula Ia, Formula Ib, Formula IIa, metal complexes thereof, or pharmaceutically acceptable salts, prodrugs and hydrates thereof. In some embodiments, the pharmaceutical composition comprises at least about 0.1 mg of a compound of Formula I, Formula II, Formula Ia, Formula Ib, Formula IIa, metal complexes thereof, or pharmaceutically acceptable salts, prodrugs and hydrates thereof. In some embodiments, the pharmaceutical composition comprises at least about 1 mg of a compound of Formula I, Formula II, Formula Ia, Formula Ib, Formula IIa, metal complexes thereof, or pharmaceutically acceptable salts, prodrugs and hydrates thereof. In some embodiments, the pharmaceutical composition comprises at least about 10 mg of a compound of Formula I, Formula II, Formula Ia, Formula Ib, Formula IIa, metal complexes thereof, or pharmaceutically acceptable salts, prodrugs and hydrates thereof. In some embodiments, the pharmaceutical composition comprises at least about 50 mg of a compound of Formula I, Formula II, Formula Ia, Formula Ib, Formula IIa, metal complexes thereof, or pharmaceutically acceptable salts, prodrugs and hydrates thereof. In some embodiments, the pharmaceutical composition comprises at least about 100 mg of a compound of Formula I, Formula II, Formula Ia, Formula Ib, Formula IIa, metal complexes thereof, or pharmaceutically acceptable salts, prodrugs and hydrates thereof. In some embodiments, the pharmaceutical composition comprises at least about 200 mg of a compound of Formula I, Formula II, Formula Ia, Formula Ib, Formula IIa, metal complexes thereof, or pharmaceutically acceptable salts, prodrugs and hydrates thereof. In some embodiments, the pharmaceutical composition comprises at least about 500 mg of a compound of Formula I, Formula II, Formula Ia, Formula Ib, Formula IIa, metal complexes thereof, or pharmaceutically acceptable salts, prodrugs and hydrates thereof. In some embodiments, the pharmaceutical composition comprises at least about 600 mg of a compound of Formula I, Formula II, Formula Ia, Formula Ib, Formula IIa, metal complexes thereof, or pharmaceutically acceptable salts, prodrugs and hydrates thereof. In some embodiments, the pharmaceutical composition comprises at least about 750 mg of a compound of Formula I, Formula II, Formula Ia, Formula Ib, Formula IIa, metal complexes thereof, or pharmaceutically acceptable salts, prodrugs and hydrates thereof. In some embodiments, the pharmaceutical composition comprises at least about 1,000 mg of a compound of Formula I, Formula II, Formula Ia, Formula Ib, Formula IIa, metal complexes thereof, or pharmaceutically acceptable salts, prodrugs and hydrates thereof.

The compositions of the present invention may be administered orally, preferably as a solid oral dose, and more preferably as a solid oral dose that may be a capsule or tablet. In preferred embodiments, the compositions of the present invention may be formulated as tablets for oral administration. In some embodiments, the composition is a solid oral dosage form, and in some embodiments, the composition is a liquid solution dosage form.

Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, illustrative examples of antimicrobial compositions for in vivo administration can be provided as solutions, especially aqueous solutions, or alcoholic solutions. Such solutions can be especially convenient for oral administration, and can also be formulated for parenteral administration, for example, by intravenous, subcutaneous, intramuscular, intraperitoneally, intraocular, and the like. In some embodiments, administration of the active compounds of the present invention can be performed by admixing the compound with ethanol because of its low toxicity. Usually ethanol will be present in the minimum concentration needed to keep the components in solution. For external topical application, isopropanol can be used. Embodiments of antimicrobial compositions preferred for topical administration can be provided as, for example, emulsions, creams, or liposome dispersions, or as an ointment in a hydrophobic carrier, such as, for example, petrolatum. In some embodiments, the amount of the active compound of Formula I, Formula II, Formula Ia, Formula Ib, Formula IIa, metal complexes thereof, or pharmaceutically acceptable salts, prodrugs and hydrates thereof in a fluid, for example, a solution or inhalable solution, emulsion or semi-solid composition can range from about 0.1 to about 1,000 μg/mL, or any range or integer in between. In particular embodiments, the amount of the active compound of Formula I, Formula II, Formula Ia, Formula Ib, Formula IIa, metal complexes thereof, or pharmaceutically acceptable salts, prodrugs and hydrates thereof is less than 100 μg/mL. In other embodiments, the amount of the active compound of Formula I, Formula II, Formula Ia, Formula Ib, Formula IIa, metal complexes thereof, or pharmaceutically acceptable salts, prodrugs and hydrates thereof is 0.1, 1, 10, 20, 30, 40, 50, 60, 70, 80, 90, 100, 110, 120, 130, 140, 150, 160, 170, 180, 190, 200, 225, 250, 275, 300, 350, 400, 450, 500, or 550 μg/mL, or any integer therebetween, for example, 1, 2, 4, 8, 16, 32, 64, 128, 256 or 512 μg/mL, or any integer therebetween.

Other possibilities also exist, including, for example, serial administration of the antimicrobial composition(s), if any. For example, in certain embodiments, the antimicrobial compositions can be constituted at the point of use, or alternatively two or more components of the compositions can be previously combined, in appropriate ratios, so that the antimicrobial compositions can be constituted at the point of use by adding the remaining components and acceptable carriers or modifying agents in appropriate ratios to achieve effective concentrations of composition components.

In some embodiments of the methods for treating, the active ingredient(s) can be administered in pro-drug forms, i.e., the active compound(s) is administered in a form which is modified within the cell to produce the functional form.

Embodiments of the antimicrobial compositions include, but are not limited to, compositions such as, for example, microemulsions, suspensions, solutions, elixirs, aerosols, and solid dosage forms. Excipients can be used in any case, and especially the case of oral solid preparations (such as, for example, powders, capsules and tablets), with the oral solid preparations being used in certain preferred embodiments. Particularly preferred oral solid preparations can be tablets.

Because of their ease of administration, tablets and capsules can represent in some embodiments the most advantageous oral dosage unit form, in which case solid pharmaceutical carriers can be preferably employed. For these embodiments, examples of suitable excipients include, but are not limited to, lactose, white sugar, sodium chloride, glucose solution, urea, starch, calcium carbonate, kaolin, crystalline cellulose and silicic acid, binders such as water, ethanol, propanol, simple syrup, glucose, starch solution, gelatine solution, carboxymethyl cellulose, shellac, methyl cellulose, potassium phosphate and polyvinyl pyrrolidone, disintegrants such as dried starch, sodium alginate, agar powder, laminaria powder, sodium hydrogen carbonate, calcium carbonate, Tween (fatty acid ester of polyoxyethylenesorbitan), sodium lauryl sulfate, stearic acid monoglyceride, starch, and lactose, disintegration inhibitors such as white sugar, stearic acid glyceryl ester, cacao butter and hydrogenated oils, absorption promoters such as quaternary ammonium bases and sodium lauryl sulfate, humectants such as glycerol and starch, absorbents such as starch, lactose, kaolin, bentonite and colloidal silicic acid, and lubricants such as purified talc, stearic acid salts, boric acid powder, polyethylene glycol and solid polyethylene glycol.

In certain embodiments, the tablet, if used, can be coated, and made into sugar-coated tablets, gelatine-coated tablets, enteric-coated tablets, film-coated tablets, or tablets containing two or more layers. If desired, tablets can be coated by standard aqueous or nonaqueous techniques.

In molding embodiments of the pharmaceutical compositions into pills, a wide variety of conventional excipients can be used. Examples include, but are not limited to, glucose, lactose, starch, cacao butter, hardened vegetable oils, kaolin and talc, binders such as gum arabic powder, tragacanth powder, gelatin, and ethanol, and disintegrants such as, for example, laminaria and agar.

In molding embodiments of the pharmaceutical compositions into a suppository form, a wide variety of conventional excipients can be used. Examples include, but are not limited to, polyethylene glycol, cacao butter, higher alcohols, gelatin, and semi-synthetic glycerides.

Other embodiments of the pharmaceutical compositions can be administered by controlled release means.

Embodiments of the pharmaceutical composition formulated into an injectable preparation can be formulated into a solution or suspension. Any conventional excipient can be used. Examples include, but are not limited to, water, ethyl alcohol, polypropylene glycol, ethoxylated isostearyl alcohol, polyoxyethylene sorbitol, and sorbitan esters. Sodium chloride, glucose or glycerol can also be incorporated into a therapeutic agent.

Embodiments of the antimicrobial compositions can contain, for example, ordinary dissolving aids, buffers, pain-alleviating agents, and preservatives, and optionally coloring agents, perfumes, flavors, sweeteners, and other drugs.

For topical application embodiments, there can be employed, as non-sprayable forms, viscous to semi-solid or solid forms comprising a carrier compatible with topical application and having a dynamic viscosity preferably greater than water. Formulations of these embodiments include, but are not limited to, solutions, suspensions, emulsions, creams, ointments, powders, liniments, salves, aerosols, etc., which can be, if desired, sterilized or mixed with auxiliary agents, e.g., preservatives, antioxidants, stabilizers, wetting agents, buffers or salts for influencing osmotic pressure, etc. For other topical application embodiments, sprayable aerosol preparations can be used wherein, for example, the active ingredient can be in combination with a solid or liquid inert carrier material.

For embodiments to be used in the disinfection of nonliving surfaces, such as, for example, countertops, surgical instruments, and bandages, antimicrobial compositions can be, for example, solutions, either aqueous or organic. For embodiments in which direct human contact with the disinfectant can be limited, such as, for example, in the disinfection of work surfaces or restrooms, mixed organic solutions can be appropriate, e.g., ethanol or isopropanol in water. Preferred alcohols for solvent purposes include, but are not limited to, ethyl, n-propyl, isopropyl, n-butyl, isobutyl, and t-butyl alcohols. Concentration of the alcohol in a mixed solvent system can range from 5% to nearly 100%. In these embodiments, there can be a cosolvent, such as, for example, be water or an aqueous buffer. In a majority of embodiments, the alcohol component can be limited to an amount necessary to keep the compounds of the present invention in solution.

In another aspect, the present invention provides a pharmaceutical formulation comprising microcrystalline cellulose in an amount from about 20% to about 50% by weight of said formulation; mannitol in about from about 10% to about 30% by weight of said formulation; crospovidone in an amount from about 2% to about 6% of said formulation; magnesium stearate in an amount from about 0.01% to about 2% of said composition; and a compound of Formula I, Formula II, Formula Ia, Formula Ib, Formula IIa, metal complexes thereof, or pharmaceutically acceptable salts, prodrugs and hydrates thereof. In some embodiments, the pharmaceutical composition comprises a diluent in an amount from about 20% to about 50% by weight of said formulation; optionally, a second diluent in an amount from about 10% to about 30% by weight of said formulation; optionally, a disintegrant in an amount from about 2% to about 6% of said formulation; optionally, a lubricant in an amount from about 0.01% to about 2% of said composition; and a compound of Formula I, Formula II, Formula Ia, Formula Ib, Formula IIa, metal complexes thereof, or pharmaceutically acceptable salts, prodrugs and hydrates thereof. In some embodiments, the pharmaceutical composition comprises microcrystalline cellulose, mannitol, croscarmellose sodium, magnesium stearate, or a combination thereof. In some embodiments, the pharmaceutically acceptable carrier comprises microcrystalline cellulose, mannitol or combination thereof; and further optionally comprises croscarmellose sodium or magnesium stearate, or combination thereof.

b. Combination Formulations

In order to increase the effectiveness of the antimicrobial composition, it may be desirable to combine these compositions and methods of the invention with a known antibacterial agent effective in the treatment or prevention of bacterial infections. Antibacterial agents and classes thereof that may be co-administered with a compound of the present invention include, without limitation, penicillins and related drugs, carbapenems, cephalosporins and related drugs, aminoglycosides, bacitracin, gramicidin, mupirocin, chloramphenicol, thiamphenicol, fusidate sodium, lincomycin, clindamycin, macrolides, novobiocin, polymyxins, rifamycins, spectinomycin, tetracyclines, vancomycin, teicoplanin, streptogramins, anti-folate agents including sulfonamides, trimethoprim and its combinations and pyrimethamine, synthetic antibacterials including nitrofurans, methenamine mandelate and methenamine hippurate, nitroimidazoles, quinolones, fluoroquinolones, isoniazid, ethambutol, pyrazinamide, para-aminosalicylic acid (PAS), cycloserine, capreomycin, ethionamide, prothionamide, thiacetazone, viomycin, eveminomicin, glycopeptide, glycylcycline, ketolides, oxazolidinone; imipenen, amikacin, netilmicin, fosfomycin, gentamicin, ceftriaxone, Ziracin, LY 333328, CL 331002, Linezolid, Synercid, Aztreonam, Metronidazole, Epiroprim, OCA-983, GV-143253, Sanfetrinem sodium, CS-834, Biapenem, A-99058.1, A-165600, A-179796, KA 159, Dynemicin A, DX8739, DU 6681; Cefluprenam, ER 35786, Cefoselis, Sanfetrinem celexetil, HGP-31, Cefpirome, HMR-3647, RU-59863, Mersacidin, KP 736, Rifalazil; Kosan, AM 1732, MEN 10700, Lenapenem, BO 2502A, NE-1530, PR 39, K130, OPC 20000, OPC 2045, Veneprim, PD 138312, PD 140248, CP 111905, Sulopenem, ritipenam acoxyl, RO-65-5788, Cyclothialidine, Sch-40832, SEP-132613, micacocidin A, SB-275833, SR-15402, SUN A0026, TOC 39, carumonam, Cefozopran, Cefetamct pivoxil, and T 3811.

In some embodiments, the composition of the present invention may precede, be co-current with and/or follow the other antibacterial agent(s) by intervals ranging from minutes to weeks. In embodiments where the antimicrobial composition of the present invention, and other antibacterial agent(s) are applied separately to a cell, tissue or organism, one would generally ensure that a significant period of time did not expire between the time of each delivery, such that the composition and antibacterial agent(s) would still be able to exert an advantageously combined effect on the cell, tissue or organism.

In some embodiments, methods for treating or preventing an infection includes administration of one or more various combination regimens comprising a therapeutically effective amount of one or more antimicrobial compounds of the present invention along with a therapeutically effective amount of one or more antibacterial agents. One of skill in the art is aware that the antimicrobial compound of the present invention and antibiotic agent can be administered in any order or combination. In other aspects, one or more antibacterial agents may be administered substantially simultaneously, or within about minutes to hours to days to weeks and any range derivable therein, prior to and/or after administering the antimicrobial compound of the present invention.

c. Administrations

The present invention relates to a method of treating or preventing a microbial infection in a mammalian subject (e.g., a human patient). In this aspect of the invention, methods are provided for inhibiting microbial cell growth. Methods encompassed in the present invention comprises administering one or more doses of one or more of the compounds of Formula I, Formula II, Formula Ia, Formula Ib, Formula IIa, metal complexes thereof, or pharmaceutically acceptable salts, prodrugs and hydrates thereof such that the compound of Formula I, Formula II, Formula Ia, Formula Ib, Formula IIa, metal complexes thereof, or pharmaceutically acceptable salts, prodrugs and hydrates thereof will contact the microbial cells in vivo, and reduce the growth and/or activity of the microorganism. Effective doses of any one or more compounds of Formula I, Formula II, Formula Ia, Formula Ib, Formula IIa, metal complexes thereof, or pharmaceutically acceptable salts, prodrugs and hydrates thereof are administered to a subject in need of such therapy. In embodiments for treating in vivo infections, the antimicrobial compositions can be administered as an active ingredient either internally or externally. For external administration, the antimicrobial compositions can be used to treat, for example, infections of the skin or mucosal surfaces, corneas, infected cuts, burns, or abrasions, bacterial skin infections, or fungal infections (e.g., athlete's foot). For internal administration, the antimicrobial compositions can be useful for treating, for example, systemic bacterial infections, especially Escherichia coli, Staphylococcus sp. and Streptococcus sp. infections. Antimicrobial compositions can also be administered internally by topical administration to mucosal surfaces, such as, for example, nasal, throat, ocular, and vaginal mucosa, for treatment of infections, particularly bacterial and yeast infections.

The compositions will include a conventional pharmaceutical carrier or excipient and a compound of the invention as the/an active agent, and, in addition, may include carriers and adjuvants, etc.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

The choice of formulation depends on various factors such as the mode of drug administration (e.g., for oral administration, formulations in the form of tablets, pills or capsules) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One specific route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound of the present invention is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds of the present invention in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Compressed gases may be used to disperse a Compound of this invention in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of the invention, or a pharmaceutically acceptable salt, prodrug or hydrate thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt, prodrug or hydrate thereof, for treatment or prevention of an infection in accordance with the teachings of this invention.

The compounds of the invention, or their pharmaceutically acceptable salts or solvates or prodrugs thereof, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of Formula I, Formula II, Formula Ia, Formula Ib, Formula IIa, metal complexes thereof, or pharmaceutically acceptable salts, prodrugs and hydrates thereof of the present invention can be administered to a patient at dosage levels in the range of about 0.1 to about 5,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art. In some embodiments, the administration of one or more doses of the compound or compounds of Formula I, Formula II, Formula Ia, Formula Ib, Formula IIa, metal complexes thereof, or pharmaceutically acceptable salts, prodrugs and hydrates thereof of the present invention, provides a plasma concentration in the subject ranging from about 0.1 μg/mL to about 100 μg/mL.

If formulated as a fixed dose, such combination products employ the compounds of this invention within the dosage range described above and the other pharmaceutically active agent(s) within its approved dosage range. Compounds of the instant invention may alternatively be used sequentially with known pharmaceutically acceptable agent(s) when a combination formulation is inappropriate.

### D. Methods for Determining Antimicrobial Activity

In some embodiments, the compounds of Formula I, Formula II, Formula Ia, Formula Ib, Formula IIa, metal complexes thereof, or pharmaceutically acceptable salts, prodrugs and hydrates thereof can be used in various antimicrobial tests to determine their antimicrobial activity for various purposes. In one embodiment, the Minimal Inhibitory Concentration (MIC) of the compounds can be determined for calculation of therapeutically effective dosages useful in the various methods described herein.

a. Dilution Methods

MIC determinations of the compounds of Formula I, Formula II, Formula Ia, Formula Ib, Formula IIa, metal complexes thereof, or pharmaceutically acceptable salts, prodrugs and hydrates thereof can be performed according to Clinical and Laboratory Standards Institute (CLSI) M7-A7 (2006) broth microdilution methods. Unless otherwise indicated, MIC values are provided in units of micrograms per milliliter.

The broth dilution method involves subjecting the isolate to a series of concentrations of antimicrobial agents in a broth environment. Microdilution testing uses about 0.05 to 0.1 ml total broth volume and can be conveniently performed in a microtiter format. Macrodilution testing uses broth volumes at about 1.0 mL in standard test tubes. For both of these broth dilution methods, the lowest concentration at which the isolate is completely inhibited (as evidenced by the absence of visible bacterial growth) is recorded as the MIC. The MIC is thus the minumum concentration of the antibiotic that will inhibit this particular isolate. The test is only valid if the positive control shows growth and the negative control shows no growth.

A procedure similar to broth dilution is agar dilution. Agar dilution method follows the principle of establishing the lowest concentration of the serially diluted antibiotic concentration at which bacterial growth is still inhibited.

b. Diffusion Methods

Because of convenience, efficiency and cost, the disk diffusion method is probably the most widely used method for determining antimicrobial resistance in clinical laboratories and private veterinary clinics.

A growth medium, usually Mueller-Hinton agar, is first evenly seeded throughout the plate with the isolate of interest that has been diluted at a standard concentration (approximately 1 to 2×10⁸ colony forming units per mL). Commercially prepared disks, each of which are pre-impregnated with a standard concentration of a particular antibiotic, are then evenly dispensed and lightly pressed onto the agar surface. The test antibiotic immediately begins to diffuse outward from the disks, creating a gradient of antibiotic concentration in the agar such that the highest concentration is found close to the disk with decreasing concentrations further away from the disk. After an overnight incubation, the bacterial growth around each disc is observed. If the test isolate is susceptible to a particular antibiotic, a clear area of “no growth” will be observed around that particular disk.

The zone around an antibiotic disk that has no growth is referred to as the zone of inhibition since this approximates the minimum antibiotic concentration sufficient to prevent growth of the test isolate. This zone is then measured in mm and compared to a standard interpretation chart used to categorize the isolate as susceptible, intermediately susceptible or resistant. MIC measurement cannot be determined from this qualitative test, which simply classifies the isolate as susceptible, intermediate or resistant.

### E. Treatment and Prevention

In some illustrative embodiments, the antimicrobial compositions useful in the present methods can be used in methods for treating in vivo infections, promoting health in animals, especially mammals, by killing or inhibiting the growth of harmful microorganisms, disinfecting surfaces, for coatings on medical devices, and protecting materials from the harmful effects of microbial contaminants. For example, in some embodiments, the antimicrobial compositions can be used in methods for disinfecting surfaces and materials, including, but not limited to, food preparation surfaces, hospital furniture and equipment, diagnostic and biomedical devices, for example, blood analysis devices, bandages, bodily appliances, catheters, and surgical instruments. In some embodiments, the antimicrobial compounds of the present invention find utility as preservatives to inhibit or prevent growth of microorganisms during manufacturing and in finished products, preservatives to inhibit or prevent growth of microorganisms in food and beverage products, stand-alone antimicrobials for direct food contact (e.g., produce wash), cosmeceuticals for promotion of skin health care, hard surface sanitation and disinfection, application to carcasses for the control of microorganisms, environmental remediation (e.g., mold and mildew), antibiotic synergism (resistance reversal), stand-alone antimicrobials for human and animal health care (topical, injectable, oral, pulmonary delivery), and decontamination of infectious biowarfare agents. In other embodiments, the antimicrobial compositions can be used in methods for combating resistant microorganisms. As used herein, the term “infection” shall be taken to mean the invasion, development and/or multiplication of a microorganism within or on another organism. An infection may be localized to a specific region of a subject or systemic. Infections for which a compound of the invention are useful for treating include any infection caused by a bacteria, a virus, a fungus or a protozoan and will be apparent to the skilled artisan from the disclosure herein.

a. In Vivo Treatment Of Microbial Infection

In the case of a systemic infection or a localized infection of a tissue or part thereof that is within a subject, caused by a bacterium, a virus, a fungus, or a protozoan, a compound or mixture of compounds of the present invention, or a combination of a compounds of the present invention and an antibiotic known in the art may be administered by, for example, perorally (e.g. a tablet, a capsule, a pill, micro-tablets and the like, or parenterally, for example, intravenous administration, intraperitoneal administration, or subcutaneous administration. In such a case, it is preferable to administer a compound or mixture of compounds of the present invention with reduced toxicity or side-effects. Preferably, the use of the compound or mixtures of compounds of the present invention are well tolerated. The use of the compounds for the treatment and prophylaxis of microbial infections, for example, antibacterial use can be performed in accordance to medical standards and guidelines promulgated and developed in collaboration with the American Academy of Pediatrics and members of the American Academy of Family Physicians, American College of Physicians, Infectious Diseases Society of America, the America College of Emergency Physicians and the Centers for Disease Control and Prevention (CDC), and, as a consequence, such use will provide therapeutic/prophylactic benefit.

The compounds of the present invention can be administered to a patient at dosage levels in the range of about 0.001 to about 10,000 mg per day. For a normal human adult having a body weight of about 70 kilograms, a dosage in the range of about 0.001 to about 1,000 mg per kilogram of body weight per day is an example. In some embodiments, the therapeutically effective amount of a compound of Formula I, Formula II, Formula Ia, Formula Ib, Formula IIa, metal complexes thereof, or pharmaceutically acceptable salts, prodrugs and hydrates thereof for treatment or prevention of a microbial infection may preferably be about 0.001 mg to about 5,000 mg, preferably from about 0.01 mg to about 4,000 mg, preferably about 1 mg to about 3,000 mg, preferably from about 300 mg to about 1,500 mg, more preferably from about 500 mg to about 1,000 mg. In some embodiments, therapeutically effective amount of a compound of Formula I, Formula II, Formula Ia, Formula Ib, Formula IIa, metal complexes thereof, or pharmaceutically acceptable salts, prodrugs and hydrates thereof per dose or per day, may be about from about 0.001 mg to about 5,000 mg, from about 0.1 mg to about 5,000 mg, from about 1 mg to about 5,000 mg, from about 10 mg to about 5,000 mg, from about 100 mg to about 5,000 mg, from about 200 mg to about 5,000 mg, from about 300 mg to about 5,000 mg, from 450 mg to about 5,000 mg, from about 0.01 mg to about 3,000 mg, from about 0.05 mg to about 3,000 mg, from about 0.1 mg to about 3,000 mg, from about 1 mg to about 3,000 mg, from 5 mg to about 3,000 mg, from about 15 mg to about 1,000 mg, from about 0.02 mg to about 1,000 mg, from about 0.1 mg to about 1,000 mg, from about 1 mg to about 1,000 mg, from about 10 mg to about 1,000 mg, from about 50 mg to about 1,000 mg, or from 100 mg to about 1,000 mg. In some embodiments, the amount of a compound of Formula I, Formula II, Formula Ia, Formula Ib, Formula IIa, metal complexes thereof, or pharmaceutically acceptable salts, prodrugs and hydrates thereof is from about 0.1 mg to about 300 mg. This dose may be administered as a single daily dose, or may be divided into several doses administered throughout the day, for example, 1 to 5 doses per day, preferably two to three doses per day.

Notwithstanding the relatively broad spectra of activity of the compounds of Formula I, Formula II, Formula Ia, Formula Ib, Formula IIa, metal complexes thereof, or pharmaceutically acceptable salts, prodrugs and hydrates thereof described herein their in vitro antimicrobial activities, for example, with respect to bacteria, expressed as minimum inhibitory concentration (MIC) or the minimum bactericidal concentration (MBC), are important considerations when selecting a compound for a particular treatment context. This is because efficacy of a compound for any particular treatment context requires a good affinity of the compound to specific binding sites in the microbe at a critical concentration and for a sufficient period of time. The pharmacokinetic properties of the compounds of the present invention can determine a critical concentration at the site of infection as well as the duration of in-vivo exposure. These concentrations and pharmacokinetic parameters such as Cₘₐₓ, Cₘᵢₙ, C₍ₘₐₓ₎ₛₛ, AUC₀₋₂₄, AUC_{0-∞}, Tₘₐₓ, and T_{1/2} can be determined through clinical trials and other experimentally controlled administrations that can serve to calculate appropriate dosages for treatment. Other factors, e.g., in-vivo disposition of the drug may affect the compound-microbe interaction in a clinical setting. The integration of these pharmacokinetic characteristics and the microbiologic activity of an antimicrobial compounds of the present invention define the pharmacodynamic parameters that form the basis for the optimal method of administration and will enhance its clinical efficacy.

Kinetics of bacterial killing are a function of the period of time required for efficacy and the MIC of the antimicrobial compound. Accordingly, it is preferred to administer an antimicrobial compound of Formula I, Formula II, Formula Ia, Formula Ib, Formula IIa, metal complexes thereof, or pharmaceutically acceptable salts, prodrugs and hydrates thereof of the present invention for a minimum period of time of 6-12 hours and/or at a concentration in target tissue (e.g., skin, serum, etc.) of at least about 2-20 times the MIC of the bacteria, preferably at least about 5 times the MIC of the bacteria or at least about 10 times the MIC of the bacteria. The time between doses may also affect efficacy of treatment, and it is preferred to administer the antimicrobial compound of the present invention such that serum antimicrobial compound levels exceed MIC by at least about 4 times during at least 60-70% of the dose interval, achievable e.g., by daily, b.i.d., q.i.d. or more frequent dosing, by dosing at higher concentration and at longer time intervals or by continuous infusion following a bolus dose to obviate any observed lag period required to reach a steady state by constant infusion.

Post-antibiotic effect (PAE) of the antimicrobial compounds of the present invention i.e., the time period after an exposure to and removal of an antimicrobial compound of Formula I, Formula II, Formula Ia, Formula Ib, Formula IIa, metal complexes thereof, or pharmaceutically acceptable salts, prodrugs and hydrates thereof during which inhibition of bacterial growth persists, may also vary for different compound-microbe interactions. This may, to a certain extent, be dependent upon the concentration of the compound of Formula I, Formula II, Formula Ia, Formula Ib, Formula IIa, metal complexes thereof, or pharmaceutically acceptable salts, prodrugs and hydrates thereof administered and/or the duration of exposure to the compound and/or the antimicrobial combination being administered.

The maximum or peak serum level (Cₘₐₓ or Peak) integrated with the MIC or MBC may define the time exposure threshold of an antimicrobial compound of the present invention. These parameters are expressed as the ratio of peak or maximal serum concentration to MIC (Cₘₐₓ/MIC), the ratio of the area under the concentration time curve (AUC) to the MIC (AUIC), and the time in which serum levels exceed the MIC (time>MIC). These parameters are studied to determine which correlate best with antimicrobial efficacy for different antimicrobial compounds. Preferred determinants of successful outcome are selected from the group consisting of peak plasma level (e.g., as determined by stepwise logistic regression taking into account significant pharmacokinetic, clinical and microbial factors), mean geometric MIC, maximal peak, mean peak/MIC, and maximal peak/MIC. Preferred Cₘₐₓ target of 10×MIC should provide at least about 90% efficacy, combined with maintenance of maximal serum level of an antimicrobial compound of Formula I, Formula II, Formula Ia, Formula Ib, Formula IIa, metal complexes thereof, or pharmaceutically acceptable salts, prodrugs and hydrates thereof to prevent the emergence of resistant mutants.

Preferably, these parameters are not inconsistent with an effective compound concentration in the target tissue in the microgram range, preferably about 1-500 μg/mL. Such dosage concentrations generally lend themselves to formulations comprising the antimicrobial compound at relatively low concentration, preferably less than about 1-100 mg/mL, and more preferably at sub-milligram concentration, even assuming high turnover of 99% in the first 12 hours following administration. As can be anticipated by one of ordinary skill in the art, the actual therapeutic doses can be readily determined using existing methods of pharmaceutical drug titration to provide therapeutic efficacy with a reasonable benefit/risk ratio commensurate with the factors commonly assessed in dosage calculation using the guidance provided in the specification and common knowledge of prescribing physicians.

In some exemplary embodiments, the in vivo efficacy of an antibacterial compound of the present invention can be confirmed by any one of a number of methods known to those skilled in the art, for example, a murine model of infection is employed, such as the murine model of infection by Pseudomonas aeruginosa described, e.g., by Tang et al., Infection and Immunity, 1278-1285 (1995). This infant mouse model of P. aeruginosa pneumonia allows for the in vivo evaluation of bacterial and host factors important in the acute stages of pulmonary infection. The use of this model also provides a means to test preventative and therapeutic strategies against the acquisition of these organisms. The basic procedure is readily amenable to determining pharmokinetic data referred to in the preceding paragraphs.

In further embodiments, the methods of treatment of a subject further comprises diagnosing the subject as needing treatment for the bacterial infection prior to administering the antimicrobial composition. Such diagnostic methods are well known to those having skill in the art.

b. Non-Living Sterilization Uses

For embodiments to be used in the disinfection of nonliving surfaces, such as, for example, countertops, food storage equipment, surgical instruments, and bandages, antimicrobial compositions incorporating one or more compounds of Formula I, Formula II, Formula Ia, Formula Ib, Formula IIa, metal complexes thereof, or pharmaceutically acceptable salts, prodrugs and hydrates thereof can be, for example, solutions, either aqueous or organic. For embodiments in which direct human contact with the disinfectant can be limited, such as, for example, in the disinfection of work surfaces or restrooms, mixed organic solutions can be appropriate, e.g., ethanol or isopropanol in water. Preferred alcohols for solvent purposes include, but are not limited to, ethyl, n-propyl, isopropyl, n-butyl, isobutyl, and t-butyl alcohols. Concentration of the alcohol in a mixed solvent system can range from 5% to nearly 100%. In these embodiments, there can be a cosolvent, such as, for example, be water or an aqueous buffer. In a majority of embodiments, the organic solvent component can be limited to an amount necessary to keep the antimicrobial compound of Formula I, Formula II, Formula Ia, Formula Ib, Formula IIa, metal complexes thereof, or pharmaceutically acceptable salts, prodrugs and hydrates thereof in solution. The skilled artisan will be aware of suitable components of a composition suitable for spray application. For example the composition comprises an antimicrobial compound of Formula I, Formula II, Formula Ia, Formula Ib, Formula IIa, metal complexes thereof, or pharmaceutically acceptable salts, prodrugs and hydrates thereof, as described herein according to any embodiment and a suitable carrier, e.g., water or saline. Such a composition may also comprise, for example, a surfactant, e.g., Tween 20, preferably, a surfactant does not inhibit or reduce the antimicrobial activity of the compound.

A wide variety of applications are envisioned for the antimicrobial compositions of the present invention, including, but not limited to, nutriceuticals to enhance health, anti-oxidant/preservatives to inhibit or prevent growth of microorganisms during manufacturing and in finished products, anti-oxidant/preservatives to inhibit or prevent growth of microorganisms in food and beverage products, stand-alone antimicrobials for direct food contact (e.g., produce wash), anti-oxidant/cosmeceuticals for promotion of skin health care, hard surface sanitation and disinfection, application to carcasses for the control of microorganisms, environmental remediation (e.g., mold and mildew), and decontamination of infectious biowarfare agents.

c. Drug Coatings in Medical Devices

In some embodiments, a compound of Formula I, Formula II, Formula Ia, Formula Ib, Formula IIa, metal complexes thereof, or pharmaceutically acceptable salts, prodrugs and hydrates thereof, described herein according to any embodiment is applied to a surface of a device to prevent microbial proliferation on that surface of the device. The device is, for example, a medical device, which includes any material or device that is used on, in, or through a patient's body in the course of medical treatment (e.g., for a disease or injury). Medical devices include but are not limited to such items as medical implants, wound care devices, drug delivery devices, and body cavity and personal protection devices. The medical implants include but are not limited to urinary catheters, intravascular catheters, dialysis shunts, wound drain tubes, skin sutures, vascular grafts, implantable meshes, intraocular devices, heart valves, prosthetic devices (e.g., hip prosthetics) and the like. Wound care devices include but are not limited to general wound dressings, biologic graft materials, tape closures and dressings, and surgical incise drapes. Drug delivery devices include but are not limited to needles, drug delivery skin patches, drug delivery mucosal patches and medical sponges. The amount of compound of Formula I, Formula II, Formula Ia, Formula Ib, Formula IIa, metal complexes thereof, or pharmaceutically acceptable salts, prodrugs and hydrates thereof, generally provided by such coatings and drug delivery devices are generally therapeutically active amounts.

### EXAMPLES

### Example 1

### Synthesis of Ortho-Aminothiophenol Compounds of Formula I and II and Metal Complexes Thereof

Synthesis of Metal Complexes

The compounds of Formula I and II as described herein can be complexed to metals and metal salts. M¹ can be any transition metal, alkali metal and alkaline earth metal, for example Cu(I) and Cu(II) centers with anions such as halides (fluoride, chloride, bromide, iodide), acetate, perchlorate, tosylate. There can also be solvent coordinated to metal center such as water, ethanol, methanol, acetonitrile, dimethylsulfoxide.

M¹ can also be Zn, Al and any transition metal, alkali metal and alkaline earth metal. In an example, the below

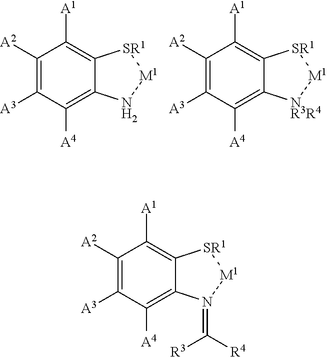

where M¹ is a transition metal, alkali metal and alkaline earth metal, for example Cu(I) and Cu(II). The compounds of Formula II with two subunits can have monometal complexes that can have chelation at one subunit or two subunits, for example, as shown below:

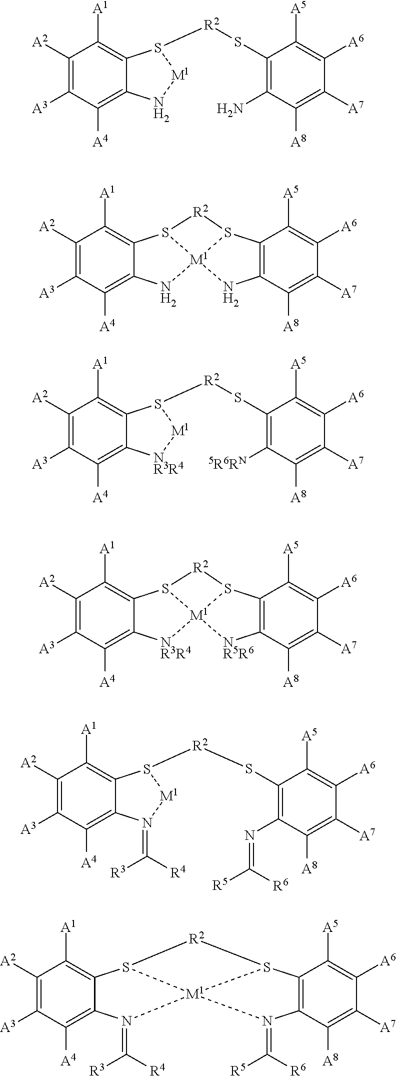

Other representative examples of Formula II compounds with metal complexes can include:

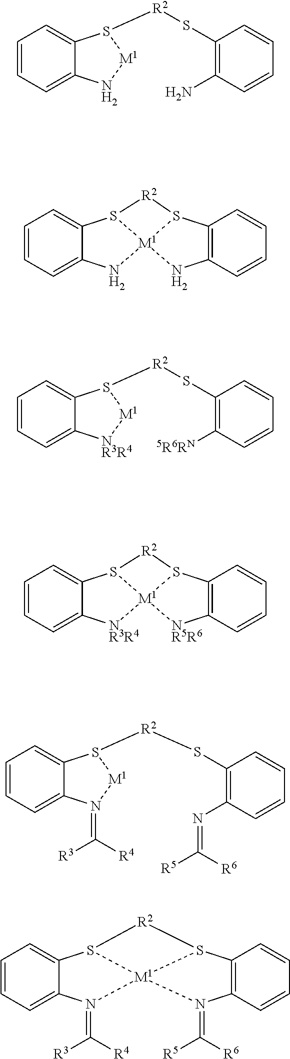

The compounds of Formula II with two subunits can have bimetal complexes with the same and different metal centers M¹ and M² as shown below:

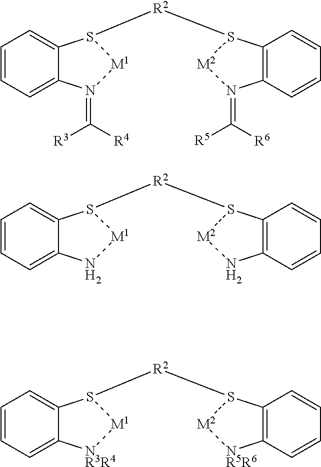

### Experimental Synthesis Procedure for Compounds

The compound numbering system for the synthesis procedure is given in scheme below:

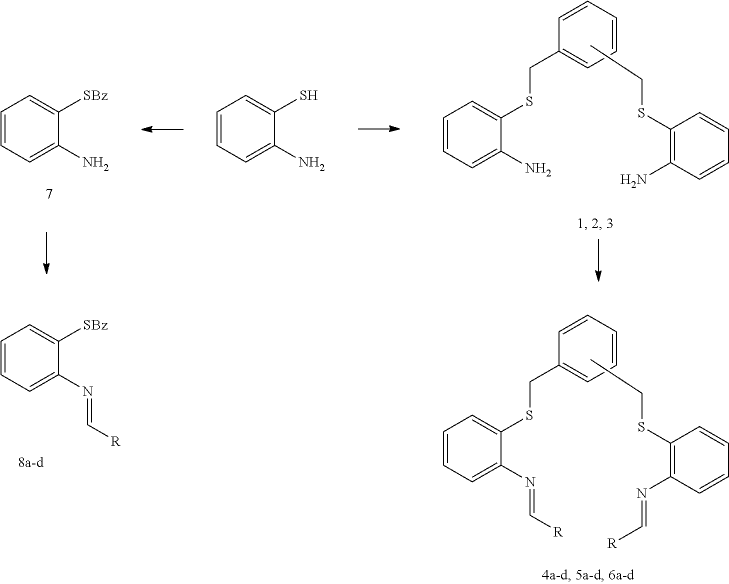

| | NH₂ | 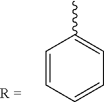 | 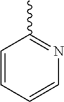 | 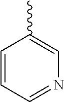 | 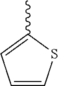 |
|---|---|---|---|---|---|
| ortho-xylene | 1 | 4a | 4b | 4c | 4d |
| meta-xylene | 2 | 5a | 5b | 5c | 5d |
| para-xylene | 3 | 6a | 6b | 6c | 6d |
| benzyl | 7 | 8a | 8b | 8c | 8d |

1a-c: Cu complexes of 1
4e: Cu complex of 4d

### α,α′-bis((o-aminophenyl)thio)-ortho-xylene (1)

In a 250 mL flask equipped with reflux condenser and under N₂ atmosphere, Na (378 mg, 16.4 mmol) was added in small portions (50 mg) to MeOH (50 mL) at r.t. After H₂ production had stopped (10 min), ortho-aminothiophenol (1.59 mL, 14.9 mmol) was added via syringe and was allowed to react for 30 min resulting in a yellow solution. α,α′-Dibromo-ortho-xylene (1.87 g, 7.08 mmol) was added and the reaction mixture was heated under refluxing condition for 5 h. The MeOH solvent was removed under reduced pressure and water (50 mL) was added and the product was extracted with diethyl ether (3×50 mL). The combined organic layers were washed with brine (50 mL), dried over anhydrous Na₂SO₄, filtered and the solvent was removed under reduced pressure affording crude product in quantitative yield. The product was dissolved in diethyl ether (50 mL) and precipitated out after treatment with anhydrous HCl gas (approx. 10 s). The precipitate was filtered and recrystallized from hot EtOH (30 mL). 1.HCl was dissolved in KOH solution (0.3 g KOH in 50 mL water) and 1 was extracted with diethyl ether (3×50 mL). The combined organic layers were washed with water (50 mL), dried over anhydrous Na₂SO₄, filtered and the solvent was removed under reduced pressure affording pure product in 92% yield (2.30 g). [152367-75-4], data congruent with literature.

Raman (solid): 3353, 3066, 3038, 1598, 1578, 1476, 1307, 1236, 1158, 1076, 1043, 1022, 842, 680 cm⁻¹.

### α,α′-bis((o-aminophenyl)thio)-meta-xylene (2)

Na (590 mg, 22.0 mmol), ortho-aminothiophenol (2.14 mL, 20.0 mmol), α,α′-dibromo-meta-xylene (2.64 g, 10.0 mmol), isolated yield (3.45 g, 98%). [354579-75-2], data congruent with literature.

Raman (oil): 3061, 1602, 1585, 1308, 1301, 1261, 1159, 1086, 1025, 1000, 833, 704, 679, 667, 558 cm⁻¹.

### α,α′-bis((o-aminophenyl)thio)-para-xylene (3)

Na (395 mg, 17.2 mmol), ortho-aminothiophenol (1.52 mL, 14.2 mmol), α,α′-dibromo-para-xylene (1.87 g, 7.09 mmol), isolated yield (2.44 g, 98%). [60786-79-0], data congruent with literature.

Raman (solid): 3059, 1602, 1450, 1314, 1230, 1199, 1117, 1080, 1021, 839, 765, 683, 549 cm⁻¹.

### S-benzyl ortho-aminothiophenol (7)

Na (319 mg, 13.9 mmol), ortho-aminothiophenol (1.30 mL, 12.2 mmol), benzyl chloride (1.33 mL, 11.6 mmol), isolated yield (2.02 g, 82%). [6325-92-4], data congruent with commercial sample.

Raman (oil): 3061, 3053, 1600, 1313, 1231, 1158, 1022, 1001, 840, 775, 689, 474 cm⁻¹.

### Large-Scale Synthesis of α,α′-bis((o-aminophenyl)thio)-ortho-xylene (1)

In a 1 L 3-necked flask equipped with thermometer and reflux condenser, Na (10.2 g, 442 mmol) was added in small 1-2 g portions to MeOH (200 mL) under N₂ atmosphere. After H₂ gas evolution ceased (45 min) and ortho-aminothiophenol (45.0 mL, 421 mmol) was added slowly and stirred for 10 min α,α′-Dibromo-ortho-xylene (58.3 g, 221 mmol) was added and the reaction mixture stirred vigorously at reflux temperature for 24 h. After cooling to room temperature (r.t.), water (500 mL) was added and product extracted with diethyl ether (5×200 mL). The combined organic layers were washed with water (200 mL), dried over anhydrous Na₂SO₄, filtered and solvent removed under reduced pressure resulting in 83% crude yield (61.2 g) containing minor impurities. 1 was recrystallized from hot MeOH (50 mL), filtered and dried in vacuo affording 78% isolated yield (57.9 g).

### Microwave Assisted Synthesis of α,α′-bis((o-aminophenyl)thio)-ortho-xylene

Na (378 mg, 16.4 mmol) was added in small portions to MeOH (10 mL) at r.t. After H₂ production ceased (10 min), ortho-aminothiophenol (1.59 mL, 14.9 mmol) was added via syringe and stirred for 5 min at r.t. α,α′-Dibromo-ortho-xylene (1.87 g, 7.08 mmol) was added and the reaction mixture heated with pulsed 300 W microwave energy and 23 bar maximum pressure for 30 min. Work-up was according to general procedure affording 91% isolated yield (2.28 g).

### General Procedure for the Imine Condensation (4a)

In a 250 mL flask equipped with reflux condenser, 1 (240 mg, 0.682 mmol) and benzaldehyde (138 μL, 1.36 mmol) were dissolved in MeOH (30 mL) and refluxed for 24 h. MeOH solvent was removed under reduced pressure and afforded 0.35 g crude product. 4a was recrystallized from hot MeOH resulting in 81% isolated yield (290 mg).

IR (KBr): 3059, 3028, 2881, 1623, 1572, 1460, 1436, 1309, 1282, 1190, 1166, 1038, 972, 881, 778 cm⁻¹.

Raman (solid): 3061, 1623, 1599, 1571, 1566, 1450, 1300, 1282, 1267, 1246, 1189, 1164, 1131, 999 cm⁻¹.

MS (EI, 70 eV): m/z (%)=92 (10), 108 (20), 184 (10), 211(100).

Anal. Calcd for C₃₄H₂₈N₂S₂: C, 77.23; H, 5.34. Found: C, 77.29; H, 5.49.

### Synthesis of 5a

2 (389 mg, 1.10 mmol), benzaldehyde (0.245 μL, 2.43 mmol), isolated yield (372 mg, 70%, >90% purity based on ¹H NMR).

IR (KBr): 3059, 3029, 2991, 2930, 2884, 1700, 1625, 1573, 1493, 1451, 1365, 1311, 1193, 1053, 761 cm⁻¹.

Raman (solid): 3060, 2901, 1624, 1599, 1572, 1461, 1449, 1442, 1363, 1189, 1167, 950, 857, 823 cm⁻¹.

MS (EI, 70 eV): m/z (%)=82 (25), 108 (85), 184 (20), 211 (100).

### Synthesis of 6a

3 (220 mg, 0.624 mmol), benzaldehyde (135 μL, 1.31 mmol), isolated yield (232 mg, 70%).

IR (KBr): 3055, 2880, 2847, 1623, 1572, 1511, 1492, 1450, 1365, 1310, 1188, 1165, 878, 848, 759 cm⁻¹.

Raman (solid): 3059, 1622, 1569, 1564, 1450, 1309, 1280, 1186, 1165, 1129, 1042, 878, 843, 772 cm⁻¹.

MS (EI, 70 eV): m/z (%)=137 (30), 152 (25), 199 (25), 277 (100).

### Synthesis of 8a

7 (914 mg, 4.25 mmol), benzaldehyde (430 μL, 4.25 mmol), isolated yield (997 mg, 78%). [91435-60-8], data congruent with literature.

Raman (solid): 3062, 1616, 1599, 1563, 1463, 1449, 1376, 1269, 1236, 1198, 1188, 1163, 999, 882 cm⁻¹.

MS (EI, 70 eV): m/z (%)=78 (65), 91 (85), 109 (20), 182 (100), 213 (100), 271 (55), 303 (15) M+.

### Synthesis of 4b

1 (334 mg, 0.948 mmol), 2-pyridine carboxaldehyde (190 μL, 1.99 mmol), isolated yield (304 mg, 61%).

IR (KBr): 3055, 3008, 1621, 1583, 1567, 1471, 1435, 1354, 1268, 1200, 1068, 1042, 992, 880, 777 cm⁻¹.

Raman (solid): 3058, 1620, 1581, 1572, 1568, 1437, 1292, 1215, 1198, 1161, 1040, 991, 879 cm⁻¹.

MS (EI, 70 eV): m/z (%)=64 (35), 91 (10), 105 (20), 183 (10), 211 (100), 349 (5), 452 (5).

### Synthesis of 5b

2 (717 mg, 2.03 mmol), 2-pyridine carboxaldehyde (413 μL, 4.32 mmol), yield after recrystallization (MeOH, 25 mL) and chromatography (hexanes/EtOAc, 2:1, Rf (5b)=0.3) on silica gel (441 mg, 41%, >90% purity based on ¹H NMR).

IR (KBr): 3054, 2922, 1711, 1627, 1586, 1567, 1500, 1472, 1316, 1265, 1198, 1088, 994, 880, 741 cm⁻¹.

Raman (neat): 3052, 2904, 1625, 1601, 1584, 1575, 1511, 1436, 1293, 1234, 1198, 1031, 1000, 795 cm⁻¹.

MS (EI, 70 eV): m/z (%)=82 (10), 108 (20), 184 (10), 211 (100).

### Synthesis of 6b

3 (405 mg, 1.15 mmol), 2-pyridine carboxaldehyde (230 μL, 2.41 mmol), isolated yield (364 mg, 60%).

IR (KBr): 3053, 3005, 2918, 1624, 1585, 1471, 1435, 1417, 1348, 1194, 1090, 1044, 993, 883, 741 cm⁻¹.

Raman (solid): 3059, 1624, 1585, 1566, 1442, 1295, 1203, 993, 845 cm⁻¹.

MS (EI, 70 eV): m/z (%)=91 (65), 136 (25), 180 (15), 214 (100), 305 (50).

### Synthesis of 8b

7 (1.08 g, 4.69 mmol), 2-pyridine carboxaldehyde (448 μL, 4.69 mmol), isolated yield (962 mg, 68%).

IR (KBr): 3059, 3029, 2920, 1621, 1585, 1563, 1493, 1473, 1281, 1067, 1038, 1026, 972, 954, 874 cm⁻¹.

Raman (solid): 3055, 1620, 1585, 1573, 1562, 1433, 1294, 1204, 1194, 1166, 1101, 991 cm⁻¹.

MS (EI, 70 eV): m/z (%)=91 (80), 109 (25), 182 (95), 215 (100), 225 (15), 271 (60), 303 (10) [M-H]⁺.

### Synthesis of 4c

1 (535 mg, 1.52 mmol), 3-pyridine carboxaldehyde (300 μL, 3.19 mmol), isolated yield (741 mg, 89%).

IR (KBr): 3060, 2934, 2860, 1623, 1585, 1571, 1563, 1474, 1461, 1438, 1325, 1183, 1025, 727, 700 cm⁻¹.

Raman (solid): 3058, 1620, 1583, 1460, 1437, 1324, 1281, 1229, 1214, 1193, 1132, 1040, 993, 788 cm⁻¹.

MS (EI, 70 eV): m/z (%)=64 (85), 108 (45), 133 (20), 211 (100), 269 (25).

### Synthesis of 5c

2 (296 mg, 0.84 mmol), 3-pyridine carboxaldehyde (181 μL, 1.93 mmol), yield after recrystallization (MeOH, 25 mL) and chromatography (hexanes/EtOAc, 2:1, Rf (5c)=0.35) on silica gel (312 mg, 70%, >90% purity based on ¹H NMR).

IR (KBr): 3055, 2924, 1624, 1605, 1587, 1478, 1447, 1419, 1323, 1204, 1159, 1083, 1025, 880, 751 cm⁻¹.

Raman (solid): 3054, 1624, 1573, 1564, 1495, 1204, 1038, 967 cm⁻¹.

MS (EI, 70 eV): m/z (%)=105 (70), 136 (15), 180 (15), 194 (20), 212 (100), 282 (20), 316 (15).

### Synthesis of 6c

3 (449 mg, 1.27 mmol), 3-pyridine carboxaldehyde (251 μL, 2.67 mmol), isolated yield (621 mg, 92%) after washing with hot MeOH (50 mL).

IR (KBr): 3061, 2920, 1622, 1586, 1572, 1512, 1474, 1436, 1325, 1272, 1183, 1069, 1026, 877, 801 cm⁻¹.

Raman (solid): 3058, 2922, 1619, 1583, 1562, 1435, 1324, 1200, 1194, 1182, 1039, 991, 877, 828 cm⁻¹.

MS (EI, 70 eV): m/z (%)=91 (15), 105 (65), 136 (30), 180 (15), 193 (15), 211 (100), 224 (15), 284 (25), 315 (55), 528 (15) [M−2H]⁺.

### Synthesis of 8c

7 (1.08 g, 5.00 mmol), 3-pyridine carboxaldehyde (470 μL, 5.10 mmol), isolated yield (1.34 g, 88%).

IR (KBr): 3058, 3025, 2915, 1623, 1584, 1494, 1473, 1462, 1372, 1324, 1183, 1069, 1025, 992, 801 cm⁻¹.

Raman (solid): 3100, 2900, 1621, 1584, 1571, 1563, 1200, 1192, 1183, 1040, 1002 cm-1.

MS (EI, 70 eV): m/z (%)=137 (30), 183 (35), 207 (40), 277 (100).

### Synthesis of 4d

1 (200 mg, 0.567 mmol), 2-thiophene carboxaldehyde (109 μL, 1.19 mmol), isolated yield (237 mg, 77%).

IR (KBr): 3095, 3056, 2864, 1609, 1571, 1564, 1462, 1424, 1268, 1193, 1068, 1042, 839, 726, 712 cm⁻¹.

Raman (solid): 3056, 1604, 1569, 1561, 1423, 1368, 1316, 1283, 1267, 1190, 1081, 1044, 1001, 956 cm⁻¹.

MS (EI, 70 eV): m/z (%)=64 (50), 84 (15), 108 (25), 207 (15), 217 (100).

Anal. Calcd for C₃₀H₂₄N₂S₄: C, 66.63; H, 4.47. Found: C, 66.85; H, 4.58.

### Synthesis of 5d

2 (400 mg, 1.13 mmol), 2-thiophene carboxaldehyde (218 μL, 2.38 mmol), isolated yield (380 mg, 62%).

IR (KBr): 3074, 1606, 1572, 1563, 1465, 1424, 1320, 1232, 1192, 1066, 1040, 841, 756, 728, 712, 579 cm⁻¹.

Raman (solid): 3054, 1605, 1572, 1563, 1464, 1424, 1320, 1223, 1191, 1079, 1040, 998, 964, 867 cm⁻¹.

MS (EI, 70 eV): m/z (%)=64 (40), 81 (15), 108 (35), 217 (100).

### Synthesis of 6d

3 (300 mg, 0.851 mmol), 2-thiophene carboxaldehyde (164 μL, 1.79 mmol), isolated yield (337 mg, 73%).

IR (KBr): 3051, 1612, 1572, 1565, 1423, 1267, 1237, 1190, 1067, 1040, 960, 853, 841, 767, 749, 716 cm⁻¹.

Raman (solid): 3055, 1614, 1571, 1565, 1422, 1236, 1198, 1190, 1158, 1079, 1040, 959, 867, 843 cm⁻¹.

MS (EI, 70 eV): m/z (%)=82 (10), 108 (15), 173 (10), 217 (100).

### Synthesis of 8d

7 (252 mg, 1.17 mmol), 2-thiophene carboxaldehyde (118 μL, 1.29 mmol), isolated yield (336 mg, 93%).

IR (KBr): 3081, 3058, 3029, 2917, 1605, 1561, 1499, 1463, 1423, 1316, 1189, 1067, 1044, 958, 840 cm⁻¹.

Raman (solid): 3056, 1604, 1569, 1561, 1462, 1423, 1369, 1316, 1283, 1267, 1190, 1082, 1044, 956 cm⁻¹.

MS (EI, 70 eV): m/z (%)=91 (60), 109 (55), 186 (45), 218 (65), 276 (100), 309 (30) M⁺.

### Large-Scale Synthesis of 6d

In a 1 L 3-necked flask equipped with reflux condenser, 3 (25.0 g, 70.9 mmol) and 2-thiophene carboxaldehyde (13.7 mL, 149 mmol) was reacted in MeOH (100 mL) under reflux for 24 h. After cooling to r.t. solids precipitated which were filtered and recrystallized from EtOH affording 96% isolated yield (36.8 g).

### Synthesis of Metal Complexes

General Procedure for Monometal Complexes with example of α,α′-bis((o-aminophenyl)thio)-ortho-xylene and Cu(ClO₄)₂

Cu(ClO₄)₂ (37.1 mg, 0.100 mmol) was dissolved in 2 mL of ethanol in a 10 mL vial at room temperature producing a blue colored solution; according to Honours thesis A. R. C. Brown 2012, Department of Chemistry, Cape Breton University. In a 25 mL round bottom flask, α,α′-bis((o-aminophenyl)thio)-ortho-xylene (35.3 mg, 0.100 mmol) was added to 3 mL absolute ethanol and heated to 50° C. for 1 h until complete dissolution, yielding a yellow colored solution. The Cu(ClO₄)₂ solution was added to the α,α′-bis((o-aminophenyethio)-ortho-xylene solution at room temperature resulting in a rapid darkening of the solution color and production of a deep blue precipitate. The reaction mixture was stirred for an additional 8 h at room temperature, before cooling to −20° C. for 16 h to increase the yield of precipitate. The precipitate was filtered, yielding 26.7 mg of a blue-black powder.

Crude yield: 26.7 mg

IR (KBr): 3442, 3250, 3053, 1603, 1560, 1475, 1093, 760, 625, 447 cm⁻¹.

### General Procedure for Dimetallic Complexes with Example of α,α′-bis((o-aminophenyl)thio)-ortho-xylene and Cu(ClO₄)₂

The same general procedure outlined above was employed for the synthesis of the binuclear Cu complex, using Cu(ClO₄)₂ (74.2 mg, 0.200 mmol) and 1 (35.3 mg, 0.100 mmol) in EtOH.

Crude yield: 100.1 mg

IR (KBr): 3448, 3253, 3066, 1616, 1475, 1088, 1082, 760, 625, 447, 438 cm⁻¹.

### α,α′-bis((o-aminophenyl)thio)-ortho-xylene and CuCl₂

The same general procedure outlined above was employed for the synthesis of the mononuclear Cu complex, using CuCl₂ (26.9 mg, 0.200 mmol) and α,α′-bis((o-aminophenyethio)-ortho-xylene (70.5 mg, 0.200 mmol) in EtOH. The red-brown powdered CuCl₂ was added to 4 mL of stirred absolute ethanol in a small vial at room temperature, dissolving within seconds to yield a bright green solution. The mixing of the two solutions resulted in a deep olive-green colored solution and the observation of a dark precipitate.

Crude yield: 43.3 mg

IR (KBr): 3415, 3161, 3057, 1585, 1477, 1304, 1238, 754, 444, 413 cm⁻¹.

### α,α′-bis((o-aminophenyl)thio)-ortho-xylene and Cu(ClO₄₂ in ACN

The same general procedure outlined above was employed for the synthesis of the mononuclear Cu complex, using Cu(ClO₄)₂ (37.1 mg, 0.100 mmol) and α,α′-bis((o-aminophenyethio)-ortho-xylene (35.3 mg, 0.100 mmol) in ACN (acetonitrile). α,α′-bis((o-aminophenyl)thio)-ortho-xylene dissolved within 3 min in stirring ACN at room temperature to yield a yellow solution. Crude yield: 31.0 mg

### α,α′-bis((o-aminophenyl)thio)-ortho-xylene and CuCl₂ in ACN

The same general procedure outlined above was employed for the synthesis of the mononuclear Cu complex, using CuCl₂ (26.9 mg, 0.200 mmol) and α,α′-bis((o-aminophenyethio)-ortho-xylene (70.5 mg, 0.200 mmol) in ACN. It was necessary sonicate the sample for the CuCl₂ to dissolve completely. Crude yield: 50.1 mg.

### α,α′-bis((o-aminophenyl)thio)-ortho-xylene and Cu(OAc)₂ in ACN

The same general procedure outlined above was employed for the synthesis of the mononuclear Cu complex, using Cu(OAc)₂ (36.3 mg, 0.200 mmol) and α,α′-bis((o-aminophenyethio)-ortho-xylene (70.5 mg, 0.200 mmol) in ACN. It was necessary to sonicate the sample for the Cu(OAc)₂ to dissolve completely to a bright blue solution. Crude yield:

6.1 mg. IR (KBr): 3427, 3049, 3008, 1583, 1458, 750, 461 cm⁻¹.

### α,α′-bis((o-aminophenyl)thio)-meta-xylene and Cu(OAc)₂ in ACN

The same general procedure outlined above was employed for the synthesis of the mononuclear Cu complex, using Cu(OAc)₂ (36.3 mg, 0.200 mmol) and α,α′-bis((o-aminophenyethio)-meta-xylene (70.5 mg, 0.200 mmol) in ACN. Crude yield: 16.9 mg. IR (KBr): 3427, 3049, 1575, 1458, 1263, 1221, 906, 738, 472, 436 cm⁻¹.

### α,α′-bis((o-aminophenyl)thio)-para-xylene and Cu(OAc)₂ in ACN

The same general procedure outlined above was employed for the synthesis of the mononuclear Cu complex, using Cu(OAc)₂ (36.3 mg, 0.200 mmol) and α,α′-bis((o-aminophenyethio)-para-xylene (70.5 mg, 0.200 mmol) in ACN. Crude yield: 21.8 mg. IR (KBr): 3427, 3049, 1574, 1446, 1263, 1221, 906, 746, 478 cm⁻¹.

### S-benzyl-ortho-aminothiophenol and Cu(OAc)₂ in ACN

The same general procedure outlined above was employed for the synthesis of the mononuclear Cu complex, using Cu(OAc)₂ (36.3 mg, 0.200 mmol) and S-benzyl-ortho-aminothiophenol (43.6 mg, 0.200 mmol) in ACN. Crude yield: 18.1 mg. IR (KBr): 3408, 3061, 1653, 1576, 1458, 1271, 1124, 904, 746, 474, 436 cm⁻¹.

### α,α′-bis((o-aminophenyl)thio)-ortho-xylene and Cu(OAc)₂ in ACN

The same general procedure outlined above was employed for the synthesis of the binuclear Cu complex, using Cu(OAc)₂ (72.6 mg, 0.400 mmol) and α,α′-bis((o-aminophenyethio)-ortho-xylene (70.5 mg, 0.200 mmol) in ACN. Crude yield: 4.8 mg.

### α,α′-bis((o-aminophenyl)thio)-meta-xylene and Cu(OAc)₂ in ACN

The same general procedure outlined above was employed for the synthesis of the binuclear Cu complex, using Cu(OAc)₂ (72.6 mg, 0.400 mmol) and 2 (70.5 mg, 0.200 mmol) in ACN. Crude yield: 18.1 mg. IR (KBr): 3423, 3363, 3057, 2914, 1579, 1450, 1263, 1221, 912, 793, 744, 478, 438 cm⁻¹.

### α,α′-bis((o-aminophenyl)thio)-para-xylene and Cu(OAc)₂ in ACN

The same general procedure outlined above was employed for the synthesis of the binuclear Cu complex using Cu(OAc)₂ (72.6 mg, 0.400 mmol) and 3 (70.5 mg, 0.200 mmol) in ACN. Crude yield: 21.4 mg. IR (KBr): 3439, 3365, 3047, 2912, 1578, 1456, 1267, 1225, 906, 786, 748, 476, 436 cm⁻¹.

### N-(2-pyridinyl methylidene) S-benzyl-ortho-aminothiophenol and Cu(OAc)₂ in ACN

The same general procedure outlined above was employed for the synthesis of the mononuclear Cu complex, using Cu(OAc)₂ (60.9 mg, 0.200 mmol) and N-(2-pyridinyl methylidene) S-benzyl-ortho-aminothiophenol (70.5 mg, 0.200 mmol) in ACN. After mixing, the initial solution was dark green in color and the formation of what appeared to be a light green precipitate was observed. The recovered retentate was light purple in color. Crude yield: 13.2 mg. IR (KBr): 3429, 3076, 3027, 2991, 1645, 1605, 1471, 1350, 1284, 1049, 850, 777, 694, 489, 420 cm⁻¹.

### NMR Experiment of the Formation of Cu (I&II) Complexes with 4d

The coordination behavior of Cu ions towards the compound of Formula II, 4d was tested by reaction with a variety of copper salts. The salts of interest were CuCl, CuBr, Cu(CO₂CH₃)₂, Cu(ClO₄)₂ and CuCl₂. In an NMR scale experiment, 2 mol equivalents of Cu salts were added to a solution of 1 mol equivalent of ligand solution in (1.5 mL) CDCl₃ (shown below). The reaction of 4d with Cu ions is instantaneous at room temperature for 10 min as the yellow colored ligand solution changed into color. The obtained complexes were characterized using NMR.

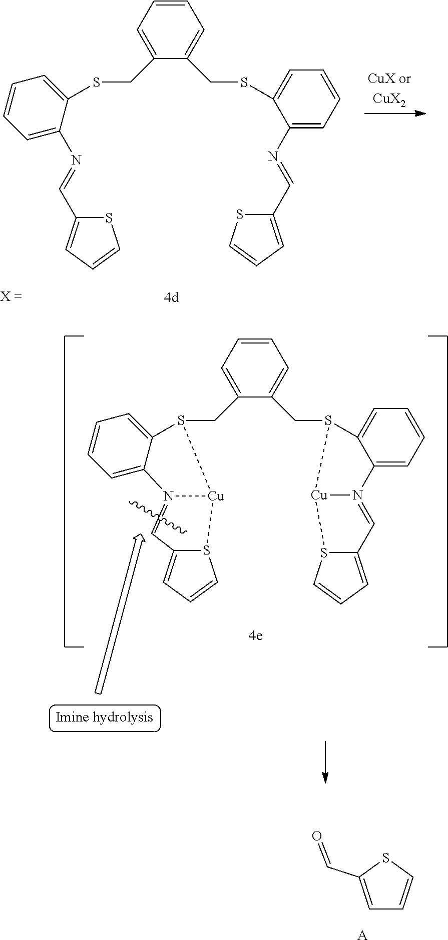

### Formation of Cu II Complex Using Ligand 1 and Cu Salts

2 mol equivalents of Cu salts (like CuCl, CuBr, Cu (CO₂CH₃)₂, and CuCl₂) were added to a solution of 1 mol equivalent of ligand 1 in (1.5 mL) CDCl₃. The reaction of 1 (α,α-bis[(o-aminophenyl)thio]-ortho-xylene) with Cu is instantaneous at room temperature and the reaction mixture was allowed to stir for 10 min as the yellow colored ligand solution changed into blue color.

### Results: Coordination of Cu Salts Towards 4d

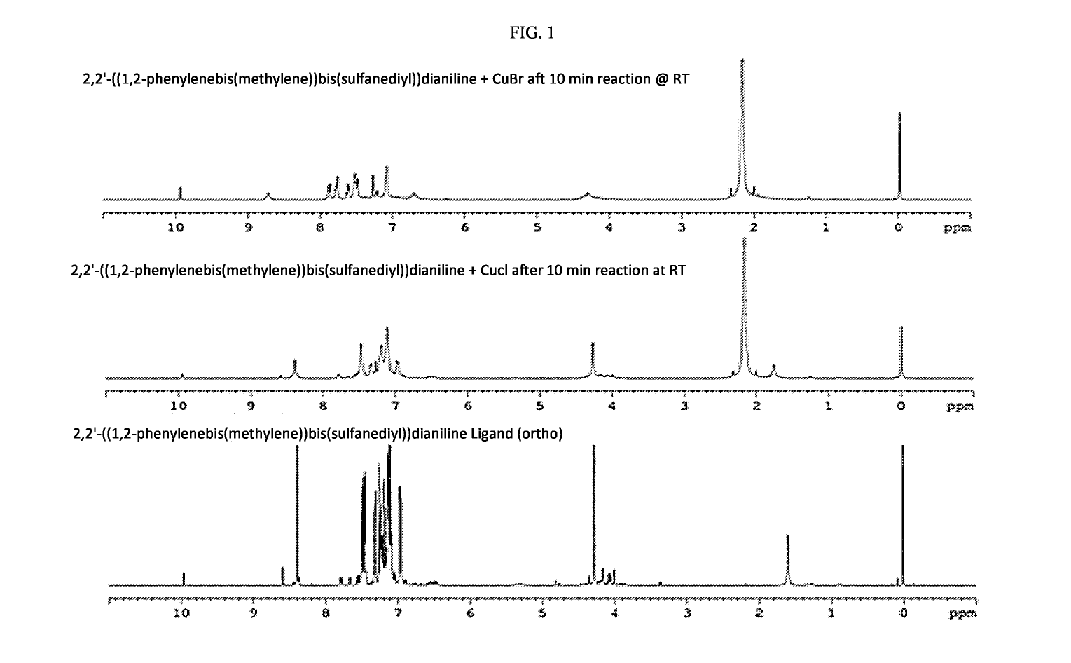

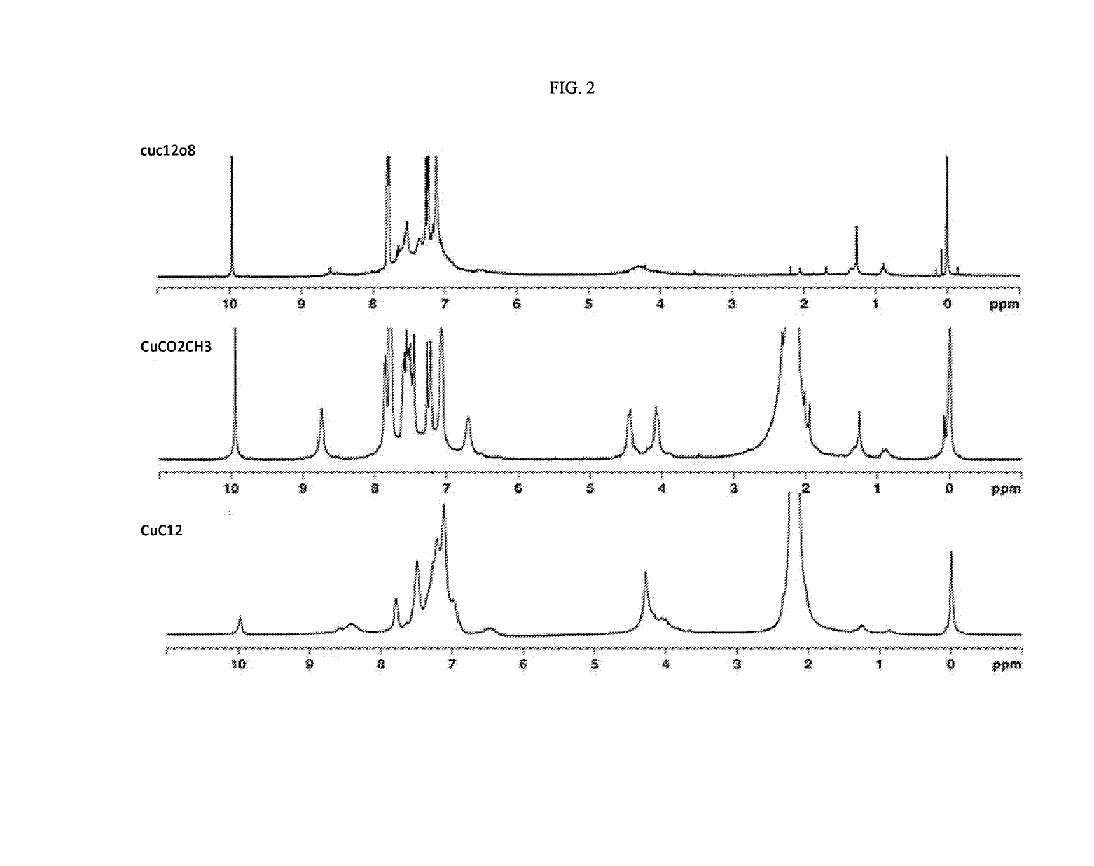

¹H NMR experiments were conducted in order to study the coordination behavior of Cu towards the thiophene pendant imine ligand 4d. At first as a control experiment, the ¹H NMR spectrum of ligand 4d was recorded in CDCl₃ solution with a concentration of 26.4 mol/L (FIG. 1). The most characteristic ¹H NMR signal is the singlet imine resonance at 8.4 ppm. The aromatic protons of the thiophene and benzene rings are observed in the 7-9 ppm region and the methylene signals are at 4.5 ppm. Therefore, we recorded the ¹H NMR spectra of the resulting Cu complexes of 4d by addition of 2 mol equivalent Cu salt to the deuterated chloroform solution. Upon addition of 2 mol equiv. CuCl, CuBr, Cu(CO₂CH₃)₂, and CuCl₂ at room temperature, an immediate color change from the yellow ligand solution to a dark brown solution with a dark colored precipitate was observed. Such a color change is typically associated with complexation behavior. The N═CH signal at 8.4 ppm completely disappeared and a large singlet signal at 10 ppm appeared showing that a hydrolyzation reaction of 4d occurred and a resulting 2-thiophene carboxaldehyde (A) was produced as 10 ppm signal is selective for aldehyde group. It could be that the color change is from complex 4e; however, such color would need to change as more aldehyde is produced. Alternative Cu complex suggestion could be the dinuclear formation of 1a as well as the mononuclear Cu complexes 3a and 11, which has not undergone deprotonation (FIG. 1). The least hydrolysis was observed with CuCl₂ as the aldehyde signal at 10 ppm is smallest (FIG. 2). In addition, the overall broadening of the ¹H NMR signals can be explained by complexation to paramagnetic Cu²⁺.

### Cu Complexes Using 1 α,α′-Bis[(o-aminophenyl)thio]-ortho-xylene

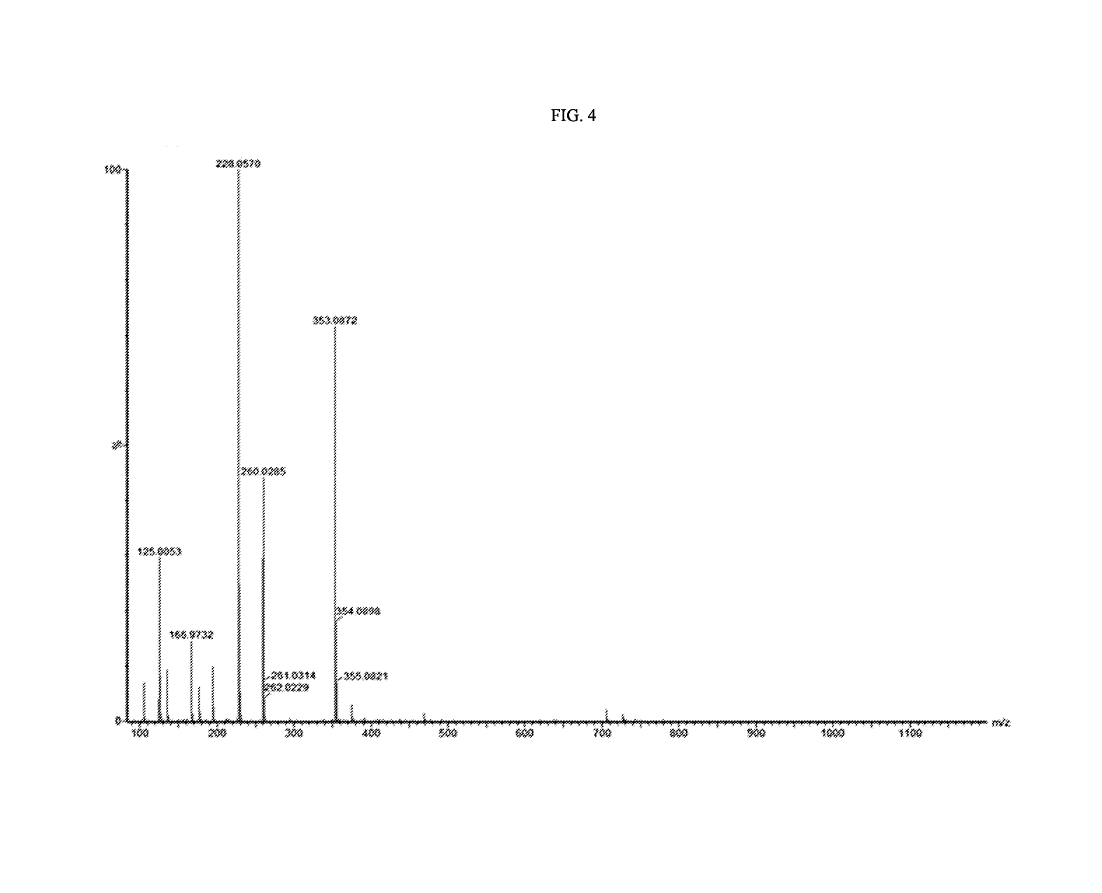

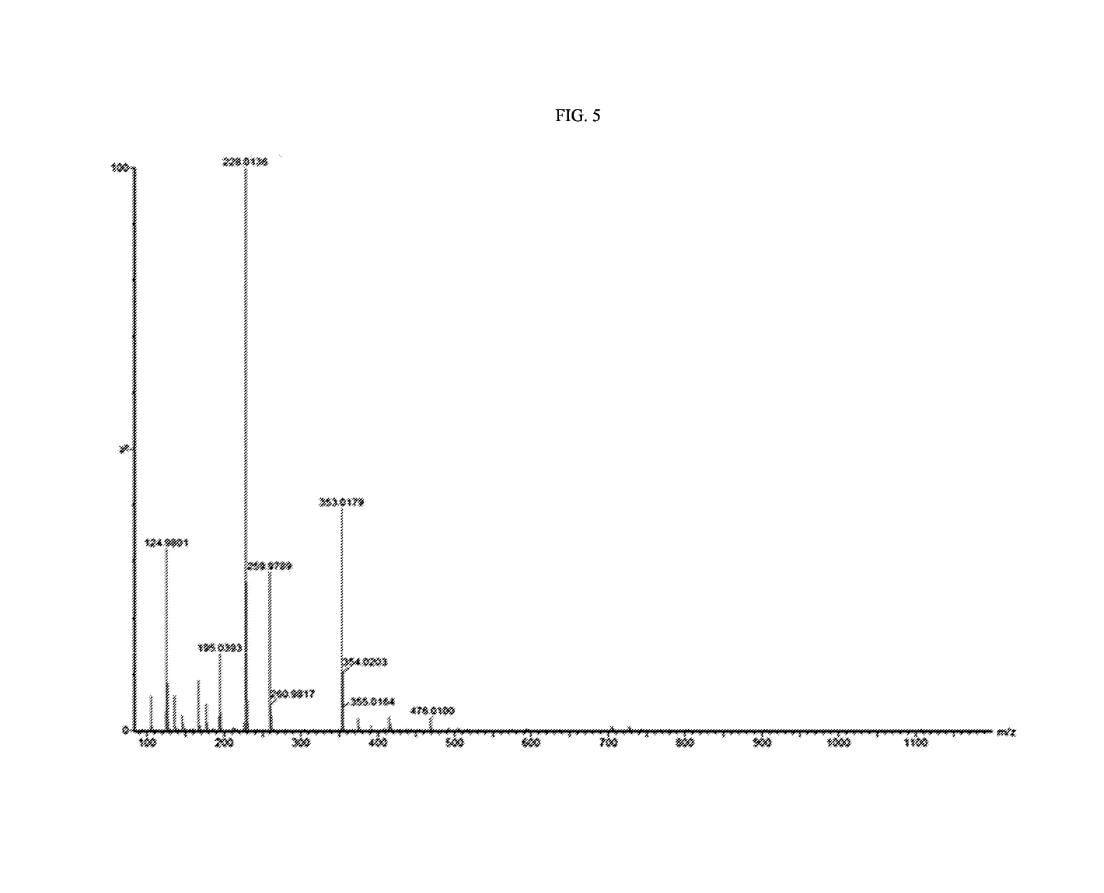

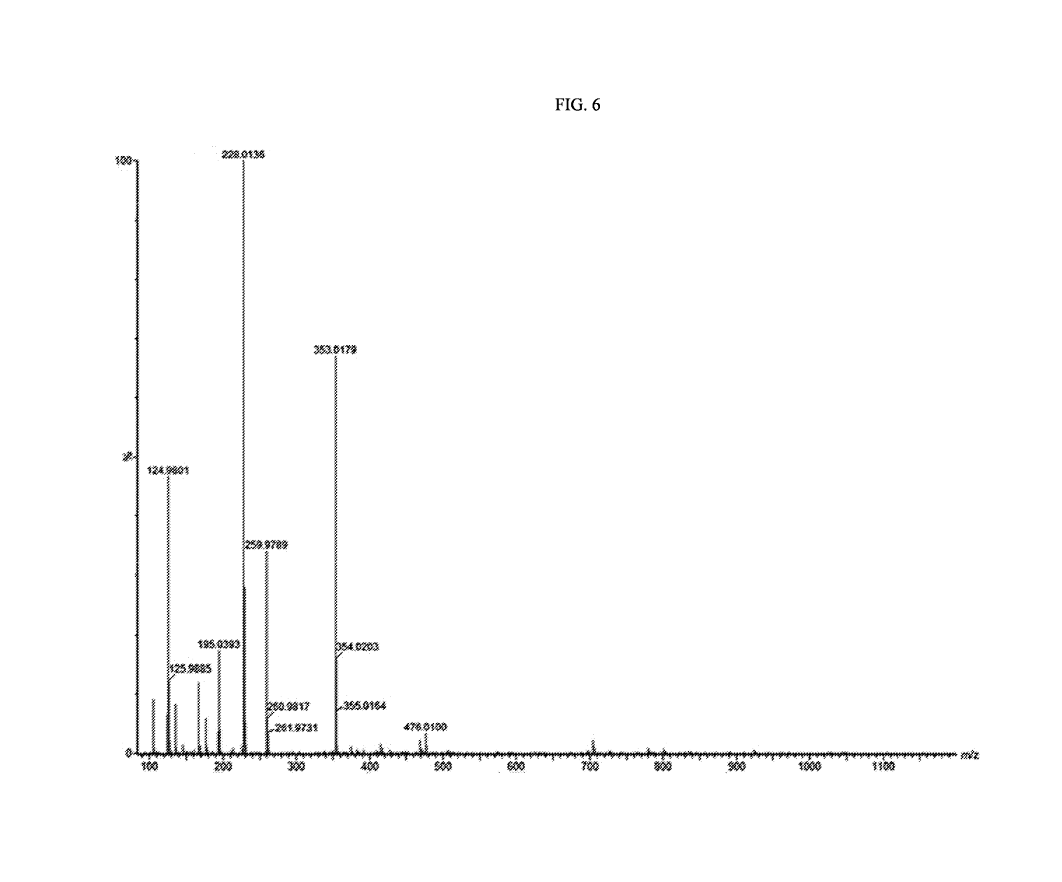

Experiments were conducted to study the coordination behavior of Cu towards the ligand 1. As 1 is produced in the hydrolysis of 4d, Cu complexation patterns were determined as shown above. At first, as a control experiment, the ¹H NMR spectrum of ligand 1 was recorded in CDCl₃ solution with a concentration of 26.4 mol/L (FIGS. 1 & 2) as well as with CuBr and CuCl salts. The characteristic ¹H NMR signal of the methylene CH₂ group is the singlet resonance at 4.29 ppm. It is expected that upon Cu coordination it is shifted. The overall boarding of the CuBr and CuCl complexes of 4 suggest that indeed complexation occurs; however, it is unclear if one or two Cu centers have been complexed. Mass spectrometric analysis was conducted using UPC-MS. Two main signals were observed a signal of 353.06 m/z that shows the M+1 signal of 1 (FIG. 4), which was protonated in the electrospray ionization environment, and 476.01 m/z which has the correct mass for 1+Cu (FIGS. 5 and 6). Higher m/z were not obtained to confirm a presence of binuclear complex. It is very likely that complex 1c has been formed in solution but further analysis is required. Nevertheless, such result is consistent with the various Cu salts and thus indicative of the high stability of the N₂S₂-tetradentate complex 1c.

### 2-(methylthio)-N-(2-thienylmethylene)-benzenamine copper (II) bromide (2-MTAtp-CuBr₂) (ECPP-129)

Proposed structure:

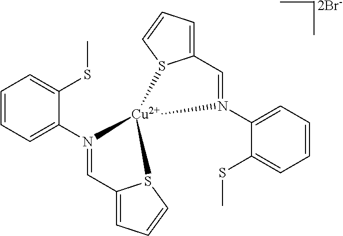

A yellow solution of 200.0 mg (0.857 mmol) of 2-MTAtp in 20 mL of toluene was added to a 25 mL brown MeOH solution containing 191.4 mg (0.857 mmol) CuBr2 with stirring at ambient temperature. The reaction mixture changed color to a dark brown/orange, and after stirring overnight some precipitation was observed. The solvent was removed under reduced pressure, followed by the addition of 20 mL of MeOH that was removed under reduced pressure leaving a dark residue. 5 mL of MeOH was added to the residue, and was sonicated for 15 mins. The undissolved material was filtered, collected and dried under vacuum yielding a black powder (245.5 mg, 83 yield). HRMS (ESI-TOF) m/z: [M]⁺ Calcd for C₂₄H₂₂CuN₂S₄ 528.9962; Found 528.9972. UV (DMF, 0.0375 mg/mL) λmax=269 (1.512), 360 (0.349).

### 2-(methylthio)-N-(2-pyridylmethylene)-benzenamine copper (I) bromide (2-MTApyr-CuBr) (ECPP-141)

Proposed Structure:

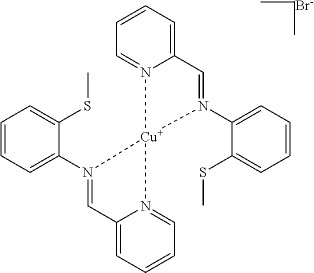

A yellow solution of 167.6 mg (0.734 mmol) of 2-MTApyr in 10 mL of toluene was added to a 20 mL green CH₃CN solution containing 53.7 mg (0.369 mmol) CuBr with stirring at ambient temperature. The reaction mixture changed color to a dark brown, and was allowed to stir for 6 h. The solvent was removed under reduced pressure, followed by the addition of 20 mL of MeOH that was removed under reduced pressure leaving a brown residue. The brown residue was dissolved in a minimal amount of dichloromethane, and was added dropwise to 20 mL of cold hexanes. The resulting brown precipitate was filtered, collected and dried under vacuum yielding a brown powder (57.9 mg, 26% yield). FTIR (KBr) 3425, 3056, 3006, 2918, 1627, 1593, 1466, 1436, 1300, 1269, 1236, 1201, 1157, 1106, 1092, 1069, 1046, 967, 958, 914, 849, 774, 767, 744, 694, 652, 567, 543, 501, 458, 417 cm-1. HRMS (ESI-TOF) m/z: [M]+ Calcd for C₁₃H₁₂CuN₂S, 291.0017; Found 290.9996. UV (DMF, 0.050 mg/mL) λmax=266 (1.020), 353 (0.208).

### 2-(methylthio)-N-(2-pyridylmethylene)-benzenamine copper (I) iodide (2-MTApyr-CuI) (ECPP-143)

Proposed Structure:

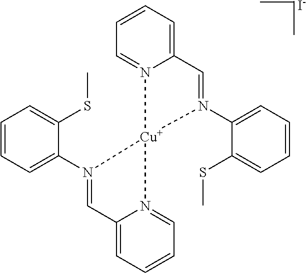

A yellow solution of 153.4 mg (0.671 mmol) of 2-MTApyr in 10 mL of toluene was added to a 20 mL colorless CH3CN solution containing 64.9 mg (0.341 mmol) CuI with stirring at ambient temperature. The reaction mixture changed color to a dark brown, and was allowed to stir for 6 h. The solvent was removed under reduced pressure, followed by the addition of 20 mL of MeOH that was removed under reduced pressure leaving a brown residue. The brown residue was dissolved in a minimal amount of dichloromethane, and was added dropwise to 20 mL of cold hexanes. The resulting brown precipitate was filtered, collected and dried under vacuum yielding a brown powder (111.2 mg, 50% yield). FTIR (KBr) 3435, 3053, 2992, 2916, 1612, 1588, 1559, 1472, 1440, 1424, 1411, 1356, 1323, 1297, 1271, 1260, 1201, 1155, 1201, 1155, 1102, 1069, 1041, 1010, 979, 957, 946, 907, 771, 740, 692, 654, 636, 570, 468, 461, 412 cm-1. HRMS (ESI-TOF) m/z: [M]+ Calcd for C₁₃H₁₂CuN₂S, 291.0017; Found 290.9996. UV (DMF, 0.050 mg/mL) λmax 266 (1.797), 362 (0.412).

### 2-(methylthio)-N-(2-pyridylmethylene)-benzenamine copper (II) bromide (2-MTApyr-CuBr2) (ECPP-145)

Proposed Structure:

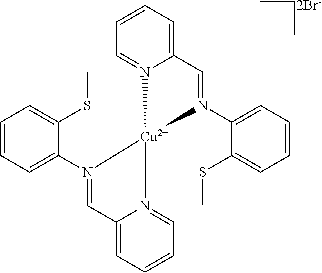

A yellow solution of 98.1 mg (0.430 mmol) of 2-MTApyr in 15 mL of 2:1 dichloromethane:methanol was added to a 5 mL brown MeOH solution containing 99.3 mg (0.444 mmol) CuBr₂ with stirring at ambient temperature. The reaction mixture changed color to a dark green, and a precipitate was observed after 30 min of stirring. After stirring overnight, the precipitate was filtered, collected and dried under vacuum yielding a green powder (121.9 mg, 42% yield). FTIR (KBr) 3435, 3077, 3060, 3005, 2951, 2937, 2920, 1613, 1593, 1560, 1479, 1441, 1415, 1300, 1274, 1236, 1204, 1157, 1107, 1041, 1018, 958, 915, 871, 847, 645, 584, 566, 543, 503, 466, 416 cm-1. HRMS (ESI-TOF) m/z: [M]+ Calcd for C₁₃H₁₂CuN₂S, 291.0017; Found 290.9996. UV (DMF, 0.025 mg/mL) λmax=269 (2.027), 365 (0.241).

### 2-(methylthio)-N-(2-phenylmethylene)-benzenamine copper (II) bromide (2-MTAtp-CuBr₂) (ECPP-167)

Proposed Structure:

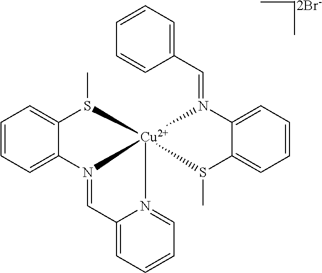

A yellow solution of 128.2 mg (0.564 mmol) of 2-MTAPh in 20 mL of toluene was added to a 20 mL brown MeOH solution containing 63.0 mg (0.281 mmol) CuBr2 with stirring at ambient temperature. The reaction mixture changed color to a dark brown/orange, and became dark brown/yellow after stiffing for 24 h. The solvent was removed under reduced pressure, followed by the addition of 10 mL of MeOH that was removed under reduced pressure orange residue. 5 mL of cold MeOH was added to the residue, and was sonicated for 5 min. The undissolved material was filtered, and dried under vacuum yielding a black solid (95.3 mg, 50% yield). FTIR (KBr) 3431, 3280, 3155, 3051, 2991, 2916, 1695, 1625, 1596, 1561, 1495, 1455, 1418, 1378, 1313, 1265, 1234, 1188, 1133, 1089, 1057, 1026, 999, 975, 962, 894, 874, 839, 813, 756, 728, 717, 689, 555, 503, 492, 465, 441 cm-1. HRMS (ESI-TOF) m/z: [M]+ Calcd for C₂₈H₂₇BrCuN₂S₂ 597.0090; Found 596.9946. UV (DMF, 0.025 mg/mL) λmax=266 (1.129), 363 (0.192) nm.

### Example 2

### Antimicrobial Activity of Compounds of Formula I and II and Metal Complexes Thereof

Introduction

The development of new resistant strains of bacteria and other organisms to current antibiotics poses a serious threat to public health. Metal complexes like copper have potential antibacterial properties which can be exploited to combat new bacteria strains (Olar R., et al. (2008) “Synthesis characterization and thermal behavior of some thiosulfato and sulfato Copper II complexes—Antibacterial activity” J. Thermal Anal Calorium 92(1) 245-251.). The antibacterial action is mediated by reaction of metal ions on carbonyl group in the peptide linkages of cell wall of bacteria leading to degradation of the protein with Cu complex. It leads to increase in oxygen concentration resulting in effective destruction of cell wall bacteria. Experimental evidence suggests that DNA loses its replication ability once the bacteria have been treated with metal complexes (Geraghty M., et al. (2000) “Synthesis and antimicrobial activity of copper (II) and manganese (II) alpha, omega-dicarboxylate complexes” Biometals 1-8. Hence, antimicrobial experiments with a selection of compounds of Formula I, Ia, Ib and Formula II, IIa, pharmaceutically acceptable salts thereof, and metal complexes thereof have been carried out on selected microorganisms in order to gauge the antibacterial properties.

Methods and Materials

The growth medium was prepared by addition of Müller-Hinton agar (38 g) to 1 L distilled water under vigorous stirring and boiled for 10 min until complete dissolution. After cooling to 25° C., the pH value of the medium was 7.3±0.1. The medium was sterilized in an autoclave at 121° C. for 15 min at 20 psi, cooled to 45-50° C. and dispended into sterile Petri dishes on a leveled, horizontal surface to give uniform depth. Stock solutions of the microorganisms were incubated at 37° C. for 24 h and the turbidity of these microbial colony were adjusted visually with sterile saline (0.85% w/v NaCl) to 0.5 McFarland standards (bioMerieux, SA (France) they are made from barium sulfate (BaSO₄). Within 15 min after adjusting the turbidity of the suspensions, the Petri dishes were inoculated using sterile cotton buds. The prepared antibiotic assay discs were placed on the surface of the seeded Müller-Hinton agar using flame sterilized forceps.

Stock solutions of compounds of Formula I, Ia, Ib and Formula II, IIa, pharmaceutically acceptable salts thereof, and metal complexes thereof were made in DMSO and were micropipetted onto the discs for 25 to 200 μg/disc. The discs were dried at ambient temperature for 30-50 min before placement onto the seeded Petri dishes which were then incubated at 37° C. for 24 h. Averaged inhibition zone diameters measurements (mm) have an accuracy of ±0.5 mm; adjacent overlapping ZOI were determined using radii measurements. Antibiotic disc like (sulfamethoxazole 23.75 μg trimethoprim 1.25 μg)/disc, Gentamicin 10 μg/disc and DMSO were used as positive and negative standards (control).

Results

Antimicrobial test was carried out to evaluate the efficacy of compounds of Formula I, Ia, Ib and Formula II, IIa, pharmaceutically acceptable salts thereof, and metal complexes thereof, as well as control tests with control compounds, against six major pathogenic bacteria namely Bacillus cereus, Pseudomonas aeruginosa, Klebsiella pneumonia, Proteus vulgaris, Staphylococcus aureus and Escherichia coli.

TABLE 2
Results of antimicrobial testing using compounds against B. cereus.

| | Amount | Zone of |
|---|---|---|
| | per disc | inhibition |
| Compound | (μg/disc) | (mm) |
| α,α′-bis((o-aminophenyl)thio)-para-xylene | 50 | 9 |
| α,α′-bis((o-aminophenyl)thio)-para-xylene | 100 | 16 |
| α,α′-bis((o-aminophenyl)thio)-para-xylene | 200 | 19 |
| S-benzyl-ortho-aminothiophenol | 166 | 12 |
| S-benzyl-ortho-aminothiophenol | 333 | 15 |
| S-benzyl-ortho-aminothiophenol | 666 | 16 |
| α,α′-bis((o-aminophenyl)thio)-ortho-xylene | 200 | 8 |
| α,α′-bis((o-aminophenyl)thio)-ortho-xylene-copper | 200 | 8 |
| (II) acetate | | |
| N-(2-pyridinyl methylidene) S-benzyl-ortho- | 75 | 7 |
| aminothiophenol | | |
| Bis [N,N′-(2-pyridinyl methylidene)]α,α′-bis | 200 | 7 |
| ((o-aminophenyl)thio)-ortho-xylene | | |
| 2-pyridine carboxaldehyde | 50 | 20 |
| 3-pyridine carboxaldehyde | 200 | 27 |
| 4-pyridine carboxaldehyde | 200 | 29 |
| Dimethyl sulfoxide | 20 μL/disc | 0 |
| Sulfamethoxazole and Trimethoprim | 23.75/1.25 | 0 |
| Gentamicin | 10 | 25 |

TABLE 3
Results of antimicrobial testing using compounds against E. coli

| | Amount | Zone of |
|---|---|---|
| | per disc | inhibition |
| Compound | (μg/disc) | (mm) |
| α,α′-bis((o-aminophenyl)thio)-ortho-xylene-copper | 200 | 7 |
| (II) chloride | | |
| α,α′-bis((o-aminophenyl)thio)-ortho-xylene-copper | 200 | 7 |
| (II) acetate | | |
| 2-pyridine carboxaldehyde | 50 | 17 |
| 3-pyridine carboxaldehyde | 200 | 24 |
| 4-pyridine carboxaldehyde | 200 | 25 |
| Dimethyl sulfoxide | 20 μL/disc | 0 |
| Sulfamethoxazole and Trimethoprim | 23.75/1.25 | 25 |
| Gentamicin | 10 | 24 |

TABLE 4
Results of antimicrobial testing using compounds against P. aeruginosa

| | Amount | Zone of |
|---|---|---|
| | per disc | inhibition |
| Compound | (μg/disc) | (mm) |
| α,α′-bis((o-aminophenyl)thio)-ortho-xylene-copper | 200 | 7 |
| (II) chloride | | |
| α,α′-bis((o-aminophenyl)thio)-ortho-xylene-copper | 200 | 7 |
| (II) acetate | | |
| 2-pyridine carboxaldehyde | 50 | 24 |
| 3-pyridine carboxaldehyde | 200 | 23 |
| 4-pyridine carboxaldehyde | 200 | 27 |
| Dimethyl sulfoxide | 20 μL/disc | 0 |
| Sulfamethoxazole and Trimethoprim | 23.75/1.25 | 20 |
| Gentamicin | 10 | 20 |

TABLE 5
Results of antimicrobial testing using compounds against P. vulgaris

| | Amount per | Zone of |
|---|---|---|
| | disc | inhibition |
| Compound | (μg/disc) | (mm) |
| α,α′-bis((o-aminophenyl)thio)-ortho-xylene-copper | 200 | 7 |
| (II) chloride | | |
| 2-pyridine carboxaldehyde | 50 | 22 |
| 3-pyridine carboxaldehyde | 200 | 21 |
| 4-pyridine carboxaldehyde | 200 | 25 |
| Dimethyl sulfoxide | 20 μL/disc | 0 |
| Sulfamethoxazole and Trimethoprim | 23.75/1.25 | 24 |
| Gentamicin | 10 | 24 |

TABLE 6
Results of antimicrobial testing using compounds against K. pnemonia

| | Amount | Zone of |
|---|---|---|
| | per disc | inhibition |
| Compound | (μg/disc) | (mm) |
| α,α′-bis((o-aminophenyl)thio)-ortho-xylene-copper | 200 | 7 |
| (II) chloride | | |
| α,α′-bis((o-aminophenyl)thio)-ortho-xylene-copper | 200 | 8 |
| (II) acetate | | |
| 2-pyridine carboxaldehyde | 50 | 23 |
| 3-pyridine carboxaldehyde | 200 | 22 |
| 4-pyridine carboxaldehyde | 200 | 26 |
| Dimethyl sulfoxide | 20 μL/disc | 0 |
| Sulfamethoxazole and Trimethoprim | 23.75/1.25 | 25 |
| Gentamicin | 10 | 25 |

TABLE 7
Results of antimicrobial testing using compounds against S. aureus

| | Amount | Zone of |
|---|---|---|
| | per disc | inhibition |
| Compound | (μg/disc) | (mm) |
| α,α′-bis((o-aminophenyl)thio)-para-xylene | 50 | 11 |
| α,α′-bis((o-aminophenyl)thio)-para-xylene | 100 | 13 |
| α,α′-bis((o-aminophenyl)thio)-para-xylene | 200 | 17 |
| S-benzyl-ortho-aminothiophenol | 166 | 11 |
| S-benzyl-ortho-aminothiophenol | 333 | 13 |
| S-benzyl-ortho-aminothiophenol | 666 | 15 |
| α,α′-bis((o-aminophenyl)thio)-ortho-xylene | 200 | 7 |
| α,α′-bis((o-aminophenyl)thio)-ortho-xylene-copper | 200 | 8 |
| (II) chloride | | |
| α,α′-bis((o-aminophenyl)thio)-ortho-xylene-copper | 200 | 12 |
| (II) acetate | | |
| N-(2-pyridinyl methylidene) S-benzyl-ortho- | 75 | 11 |
| aminothiophenol | | |
| Bis [N,N′-(2-pyridinyl methylidene)]α,α′-bis | 200 | 7 |
| ((o-aminophenyl)thio)-ortho-xylene | | |
| 2-pyridine carboxaldehyde | 50 | 15 |
| 3-pyridine carboxaldehyde | 200 | 32 |
| 4-pyridine carboxaldehyde | 200 | 30 |
| Dimethyl sulfoxide | 20 μL/disc | 0 |
| Sulfamethoxazole and Trimethoprim | 23.75/1.25 | 25 |
| Gentamicin | 10 | 25 |

### Example 3

### Antimicrobial Activity of Compounds of Formula Ia and IIa and Metal Complexes Thereof

Materials and Methods

All reagents and solvents (reagent grade) were used without further purification unless stated otherwise. UPLC grade solvents (optima label grade) were purchased from Fisher Scientific. 2-(Methylthio)aniline was purchased from Sigma Aldrich. NMR spectra were recorded on a 400 MHz Bruker Avance II spectrometer operating at 400.17 MHz for ¹H and 100.6 MHz for ¹³C. ¹H/¹³C NMR chemical shifts are reported in ppm and referenced to tetramethylsilane (δ=0 ppm) as internal standard. J values are given in Hz. UPLC-HRMS analyses were performed on a Waters Acquity Xevo G2 QToF using a C-18 column (Waters BEH C18 1.7 μm, 2.1 mm×50 mm) and ESI positive mode. Compounds were dissolved in 90 Vol % CH₃CN:10 Vol % nanopure H₂O for UPLC-HRMS analysis or directly injected using ASAP probe. Calculated theoretical isotope envelope for [M−1]⁺. UV-vis spectra were recorded in quartz cuvettes on a Varian Cary 100 Bio UV-Vis spectrometer and UV-vis spectrophotometric experiments were conducted with a Spectronic 20+D instrument by Spectronic Instruments using 0.5 in path length cuvettes (Thermo Scientific) test tubes. FTIR spectra were recorded on a Thermo Nicolet 6700 FTIR Spectrometer as KBr pellet (approximately 1.5 mg compound in 300 mg anhydrous KBr) in the 4,000 cm⁻¹ to 400 cm⁻¹ range with 2 cm⁻¹ resolution.

Sterile assay discs (6 mm diameter) were obtained from VWR Whatman and antibiotic assay discs (6 mm diameter) containing getamicin (10 μg/disc) and the combination drug sulfamethoxazole 23.75 μg/disc and trimethoprim 1.25 μg/disc were obtained from Becton, Dickinson and Company (USA). Dimethyl sulfoxide (DMSO) for microbial experiments was BioReagent™ grade and was purchased from Sigma-Aldrich. The nutrient broth and Müller-Hinton agar was purchased from Becton, Dickinson and Company (USA). Bacillus cereus, Pseudomonas aeruginosa (#27853), Klebsiella pneumonia (#13883), Proteus vulgaris (#13315), Staphylococcus aureus (#25923) and Escherichia coli (#25922) strains were obtained from the American Type Culture Collection (ATCC). All materials in contact with antimicrobial matter was treated for 40 min at 121° C. and 20 psi in a biohazard autoclave bag prior to waste disposing or washing. McFarland turbity standards containing barium sulfate were obtained from bioMérieux.

In Vitro Disc Diffusion Assays

The growth medium was prepared by addition of Müller-Hinton agar (38 g) to 1 L distilled water under vigorous stirring and boiled for 10 min until complete dissolution. After cooling to 25° C., the pH value of the medium was 7.3±0.1. The medium was sterilized in an autoclave at 121° C. for 15 min at 20 psi, cooled to 45-50° C. and dispended into sterile Petri dishes on a leveled, horizontal surface to give uniform depth. Stock solutions of the microorganisms were incubated at 37° C. for 24 h and the turbidity of the microbial colonies were adjusted visually with sterile saline (0.85% w/v NaCl) to 0.5 McFarland standards. Within 15 min after adjusting the turbidity of the suspensions, the Petri dishes were inoculated using sterile cotton buds. The prepared antibiotic assay discs were placed on the surface of the seeded Müller-Hinton agar using flame sterilized forceps.

Stock solutions of compounds and the Cu complexed compounds were made in DMSO and were micropipetted onto the discs for 25 to 200 μg compound/disc. The discs were dried at ambient temperature for 30-50 min before placement onto the seeded Petri dishes which were then incubated at 37° C. for 24 h. Averaged inhibition zone measurements (mm) have an accuracy of ±0.5 mm; adjacent overlapping ZOI were determined using radii measurements. Observed slight growth with sulfonamides/trimethoprim at the rim of the ZOI did not account in the measurements. Results of the inhibition experiments indicate that the compounds of the present invention have antibacterial activity against gram positive species Bacillus cereus and Staphylococcus aureus as shown in Tables 8-10. The activity profile of the compounds tested in Tables 8-10 can be compared to a control antibiotics Sulfamethoxazole (23.75 μg/disc), Trimethoprim (1.25 μg/disc) and Gentamicin (10 μg/disc) as shown in Table 11.

TABLE 8
Zone of inhibition (ZOI) experiments: Determination of minimum
inhibitory concentration for selected N,S-ligand.^{a}

| | ZOI (mm)^{b} | | | | | | |
|---|---|---|---|---|---|---|---|
| | L1 | L1 | L1 | L2 | L3 | L3 | L4 |
| | 50 | 100 | 200 | 200 | 50 | 75 | 200 |
| | μg/ | μg/ | μg/ | μg/ | μg/ | μg/ | μg/ |
| Organism | disc | disc | disc | disc | disc | disc | disc |
| B. cereus | 1.5 | 5 | 6.3 | 2 | 0 | 0.5 | 0.5 |
| S. aureus | 2.5 | 3.5 | 5.5 | 1.5 | 0 | 2.5 | 0.5 |

^{a}Following general method; 6 mm diameter discs.
^{b}Averaging multiple measurements;
accuracy ± 0.5 mm.^{c}

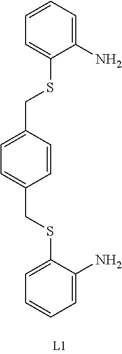 [SMILES: Nc1ccccc1SCc1ccc(CSc2ccccc2N)cc1]

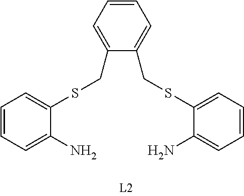 [SMILES: Nc1ccccc1SCc1ccccc1CSc1ccccc1N]

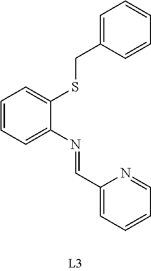 [SMILES: C(=N\c1ccccc1SCc1ccccc1)/c1ccccn1]

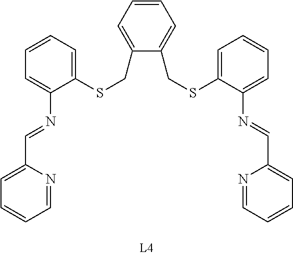 [SMILES: C(=N\c1ccccc1SCc1ccccc1CSc1ccccc1/N=C/c1ccccn1)/c1ccccn1]

TABLE 9
Summary of ZOI data collected for Cu complexes against Bacillus
cereus at 6.25 μg, 12.5 μg and 25 μg Cu.^{a}

| | Zone of Inhibition (mm) | | |
|---|---|---|---|
| Compound | 6.25 μg Cu/disc | 12.5 μg Cu/disc | 25 μg Cu/disc |
| ECPP-129 | 0 | 0.5 | 9 |
| ECPP-167 | 0 | 1 | 2 |
| ECPP-141 | 0 | 0 | 2 |
| ECPP-145 | 0 | 0 | 0.5 |

^{a}Following general method; 6 mm diameter discs. Averaging multiple measurements; accuracy ± 0.5 mm.

TABLE 10
Summary of ZOI data collected for Cu complexes against
Staphylococcus aureus at 6.25 μg, 12.5 μg and 25 μg Cu.^{a}

| | Zone of Inhibition (mm) | | |
|---|---|---|---|
| Compound | 6.25 μg Cu/disc | 12.5 μg Cu/disc | 25 μg Cu/disc |
| ECPP-129 | 5 | 7 | 10 |
| ECPP-167 | 4 | 5 | 9 |
| ECPP-141 | 0.5 | 2 | 5 |
| ECPP-143 | 0 | 0 | 1 |
| ECPP-145 | 0 | 1 | 4 |

^{a}Following general method; 6 mm diameter discs. Averaging multiple measurements; accuracy ± 0.5 mm.

TABLE 11
Zone of inhibition (ZOI) control experiments: DMSO, combination
drugs sulfamethoxazole and trimethoprim, and gentamicin.^{a}

| | ZOI (mm)^{b} | Sulfamethoxazole | |
|---|---|---|---|
| | DMSO | (23.75 μg/disc) and | |
| | (20 μL/ | Trimethoprim | Gentamicin |
| Organism | disc) | (1.25 μg/disc) | (10 μg/disc) |
| B. cereus | 0 | 0 | 9.5 |
| E. coli | 0 | 9.5 | 9 |
| P. aeruginosa | 0 | 7 | 7 |
| P. vulgaris | 0 | 9 | 9 |
| K. pnemonia | 0 | 9.5 | 9.5 |
| S. aureus | 0 | 9.5 | 9.5 |

^{a}Following general method; 6 mm diameter discs.
^{b}Averaging multiple measurements; accuracy ± 0.5 mm.

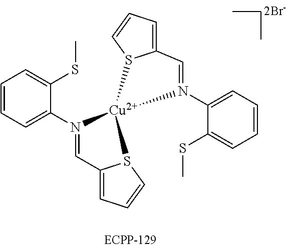

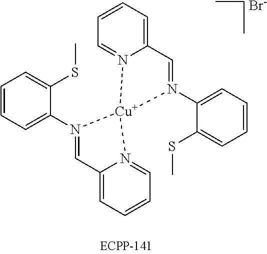

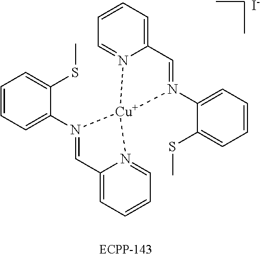

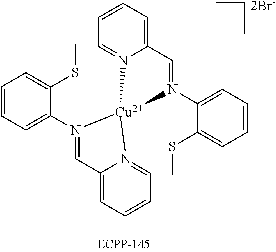

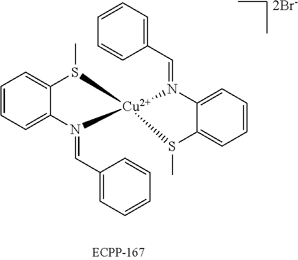

Tested compounds L1-L4, ECPP-129, ECPP-167, ECPP-141, ECPP-143, and ECPP-145 were not active against Pseudomonas aeruginosa (#27853), Klebsiella pneumonia (#13883), Proteus vulgaris (#13315), and Escherichia coli (data not shown).

### Example 4

### Antioxidant Activity of Compounds of Formula I and II and Metal Complexes Thereof

In addition to antibacterial properties, antioxidant activity was assessed in the compounds of Formula I and II and metal complexes thereof as antioxidant compounds play an important role in remediating the toxic effects of reactive oxygen species and other free radicals. The main characteristic of an antioxidant compound is its ability to trap free radicals. Highly reactive free radicals and oxygen species are present in biological systems from a wide variety of sources. Free radical damage is known to occur during bacterial and other microbial infections. Free radicals are produced by cells of the immune system during an active infection, and generally as a result of the inflammatory processes. Various antioxidant activity methods have been used to monitor and compare the antioxidant activity of compounds such as, e.g., 2,2-diphenyl-1-picryl-hydrazyl (DPPH) method.

Methods and Materials

DPPH (2,2-diphenyl-1-picryl-hydrazyl, 150 μL, 1 mM solution in methanol) was added to 3 mL methanol in a cuvette (0.5 in diameter) and UV-vis absorption recorded at 517 nm for baseline reading. In a cuvette, DPPH (150 μL, 1 mM solution in methanol) was added to a solution containing compound in 3 mL methanol. The reaction was allowed to stir at 25±1° C. for 30 min under exclusion of light. Absorbance was recorded at 517 nm against reference cuvette filled with methanol. The antioxidant activity, % Antiox-Act, was calculated based on ((absorption (baseline)−absorption (compound))/absorption (baseline). All tests were performed with three replicates and the results were averaged.

TABLE 12
Results for antioxidant tests

| | Mass of | |
|---|---|---|
| | compound | % |
| Compound | (μg) | Antiox-Act |
| α,α′-bis((o-aminophenyl)thio)-ortho-xylene | 50 | 60.8 |
| α,α′-bis((o-aminophenyl)thio)-ortho-xylene- | 50 | 56.1 |
| copper (II) chloride | | |
| α,α′-bis((o-aminophenyl)thio)-ortho-xylene- | 50 | 63.5 |
| copper (II) acetate | | |
| N-(2-pyridinyl methylidene) S-benzyl-ortho- | 50 | 65.8 |
| aminothiophenol | | |
| α,α′-bis((o-aminophenyl)thio)-para-xylene | 50 | 65.4 |
| 2-pyridine carboxaldehyde | 50 | 67.0 |
| Bis [N,N′-(2-pyridinyl methylidene)]α,α′- | 50 | 61.3 |
| bis((o-aminophenyl)thio)-ortho-xylene | | |

The embodiments and the examples described herein are exemplary and not intended to be limiting in describing the full scope of compositions and methods of the present invention. Equivalent changes, modifications and variations of some embodiments, materials, compositions and methods can be made within the scope of the present invention, with substantially similar results.

1. A method for treating or preventing a microbial infection in a subject in need thereof, the method comprising administering to the subject, a therapeutically effective amount of a compound of Formula Ia or a compound of Formula IIa, or one or two compounds independently selected from Formula Ia or Formula IIa complexed with a metal core, or a pharmaceutically acceptable salt, prodrug or hydrate thereof, 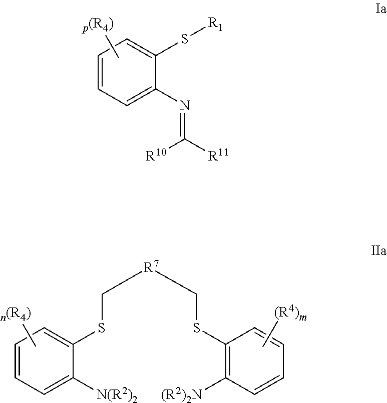
wherein,
R¹ is C₁₋₆ alkyl, C₂₋₆ alkenyl, aryl, heteroaryl, C₃₋₁₀ cycloaliphatic, or 5-10 membered heterocycloaliphatic having 1-3 heteroatoms independently selected from N, O, or S, any of which is optionally substituted;
R² is independently hydrogen, or each pair of R² groups is —N═CR¹⁰R¹¹, wherein one of R¹⁰ and R¹¹ is hydrogen and the other is optionally substituted phenyl or an optionally substituted 5-6 membered heteroaryl having 1-2 heteroatoms independently selected from N, O, or S;
R⁴ is —Z^{B}R⁶, wherein each Z^{B} is independently a bond or an optionally substituted branched or straight C₁₋₆ aliphatic chain wherein up to two carbon units of Z^{B} are optionally and independently replaced by —CO—, —CS—, —CONR^{B}—, —CO₂—, —OCO—, —NR^{B}CO₂—, —O—, —NR^{B}CONR^{B}—, —OCONR^{B}—, —NR^{B}NR^{B}—, —NR^{B}CO—, —S—, —SO—, —SO₂—, —NR^{B}—, —SO₂NR^{B}—, —NR^{B}SO₂—, or —NR^{B}SO₂NR^{B}—,
R⁶ is independently R^{B}, halo, —OH, —NH₂, —NO₂, —CN, —CF₃, or —OCF₃,
R^{B} is independently hydrogen, an optionally substituted aliphatic, an optionally substituted cycloaliphatic, an optionally substituted heterocycloaliphatic, an optionally substituted aryl, or an optionally substituted heteroaryl; or
two R⁴ groups together with the carbon atoms to which they are attached form an optionally substituted 5-6 membered ring having 0-3 heteroatoms independently selected from N, O, or S;
One of R¹⁰ and R¹¹ is hydrogen and the other is optionally substituted phenyl or an optionally substituted 5-6 membered heteroaryl having 1-2 heteroatoms independently selected from N, O, and S; and
R⁷ is an optionally substituted phenyl or an optionally substituted 6 membered heteroaryl having 1-2 heteroatoms independently selected from N, O, or S and
Each of m, n, and p is independently 0 or a positive integer from 1-3.

2. The method of claim 1, wherein the metal core is an alkali metal, an alkali earth metal, or a transition metal.

3. The method of claim 1, wherein the metal core is Cu, Ag, or Au.

4. The method of claim 3, wherein the copper metal core is Cu(I) or Cu(II) or combinations thereof.

5. The method of claim 1, wherein the compound is a compound of Formula Ia.

6. The method of claim 1, wherein R⁴ is independently H, R² is independently H, R¹⁰ is independently H, and R¹¹ is independently pyridyl, phenyl or thiophene.

7. The method of claim 6, wherein, R¹¹ is 2-pyridyl, phenyl, or 2-thiophene.

8. The method of claim 5, wherein one or two compounds of Formula Ia is/are complexed with a metal core.

9. The method of claim 8, wherein the metal core is Cu(I) or Cu(II).

10. The method of claim 1, wherein the compound is a compound of Formula IIa.

11. The method of claim 10, wherein R₇ is selected from ortho-xylylene and para-xylylene.

12. The method of claim 10, wherein one or two compounds of Formula IIa is/are complexed with a metal core.

13. The method of claim 12, wherein the metal core is Cu(I) or Cu(II).

14. The method of claim 1, wherein the compound of Formula Ia or Formula IIa, or a pharmaceutically acceptable salt thereof, is selected from:

| Compound Name | Structure |
|---|---|
| 2,2′-((1,2- phenylenebis(methylene))bis(sulfanediyl))dianiline | 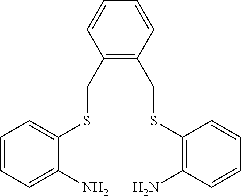 [SMILES: Nc1ccccc1SCc1ccccc1CSc1ccccc1N] |
| 2,2′-((1,4- phenylenebis(methylene))bis(sulfanediyl))dianiline | 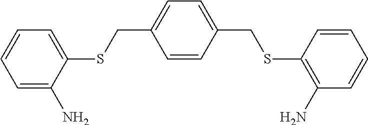 [SMILES: Nc1ccccc1SCc1ccc(CSc2ccccc2N)cc1] |
| (NZ,N′Z)-2,2′-((1,2- phenylenebis(methylene))bis(sulfanediyl))bis(N- (pyridin-2-ylmethylene)aniline) | 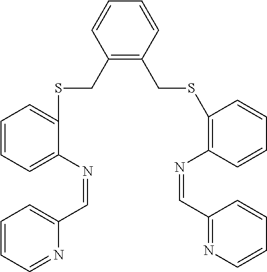 [SMILES: C(=N/c1ccccc1SCc1ccccc1CSc1ccccc1/N=C\c1ccccn1)/c1ccccn1] |
| (NZ,N′Z)-2,2′-((1,2- phenylenebis(methylene))bis(sulfanediyl))bis(N- (thiophen-2-ylmethylene)aniline) | 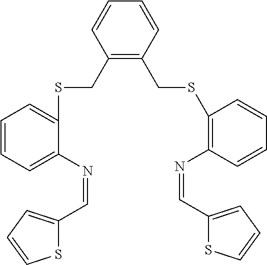 [SMILES: C(=N/c1ccccc1SCc1ccccc1CSc1ccccc1/N=C\c1cccs1)/c1cccs1] |
| (Z)-2-(benzylthio)-N-(pyridin-2-ylmethylene)aniline | 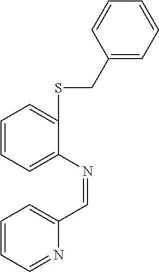 [SMILES: C(=N/c1ccccc1SCc1ccccc1)/c1ccccn1] |
| 2,2′-((1,4- phenylenebis(methylene))bis(sulfanediyl))dianiline | 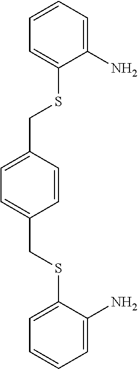 [SMILES: Nc1ccccc1SCc1ccc(CSc2ccccc2N)cc1] |
| 2,2′-((1,2- phenylenebis(methylene))bis(sulfanediyl))dianiline | 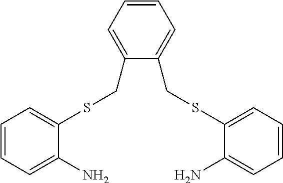 [SMILES: Nc1ccccc1SCc1ccccc1CSc1ccccc1N] |
| (E)-2-(benzylthio)-N-(pyridin-2- ylmethylene)aniline | 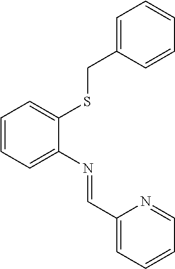 [SMILES: C(=N\c1ccccc1SCc1ccccc1)/c1ccccn1] |
| (NE,N′E)-2,2′-((1,2- phenylenebis(methylene))bis(sulfanediyl))bis(N- (pyridin-2-ylmethylene)aniline) | 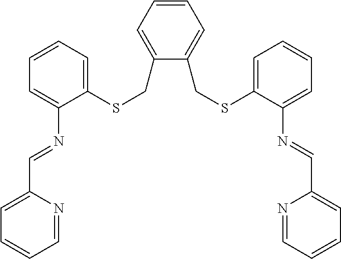 [SMILES: C(=N\c1ccccc1SCc1ccccc1CSc1ccccc1/N=C/c1ccccn1)/c1ccccn1] |
| 2-(methylthio)-N-(2-thienylmethylene)- benzenamine copper (II) bromide | 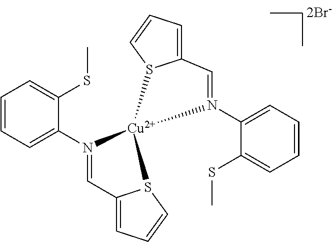 |
| 2-(methylthio)-N-(2-pyridylmethylene)- benzenamine copper (I) bromide | 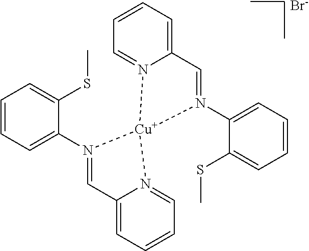 |
| 2-(methylthio)-N-(2-pyridylmethylene)- benzenamine copper (I) iodide | 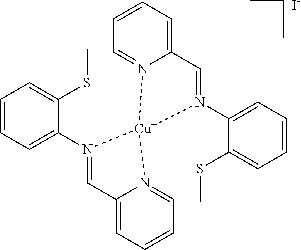 |
| 2-(methylthio)-N-(2-pyridylmethylene)- benzenamine copper (II) bromide | 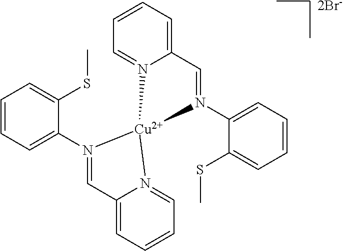 |
| 2-(methylthio)-N-(2-phenylmethylene)- benzenamine copper (II) bromide | 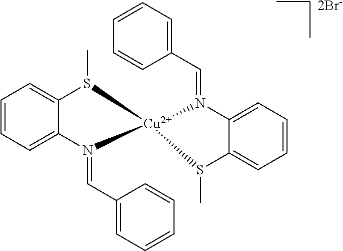 |

15. The method of claim 1, wherein the microbial infection is a bacterial infection.

16. The method of claim 15, wherein the microbial infection is a bacterial infection.

17. The method of claim 1, wherein the microbial infection is a fungal infection.

18. The method of claim 1, wherein the subject is a mammal.

19. The method of claim 20, wherein the subject is a human subject.

20. A pharmaceutical composition for use in the treatment of a microbial infection in a subject in need thereof, the pharmaceutical composition comprising a therapeutically effective amount of a compound of Formula Ia, a compound of Formula IIa, or one or two compounds independently selected from Formula Ia or Formula IIa complexed with a metal core, or a pharmaceutically acceptable salt, prodrug or hydrate thereof, or an antimicrobial composition comprising a therapeutically effective amount of a compound of Formula Ia, a compound of Formula IIa, a compound of Formula Ia or a compound of Formula IIa complexed with a metal core, or a pharmaceutically acceptable salt, prodrug or hydrate thereof, and a solvent or diluent 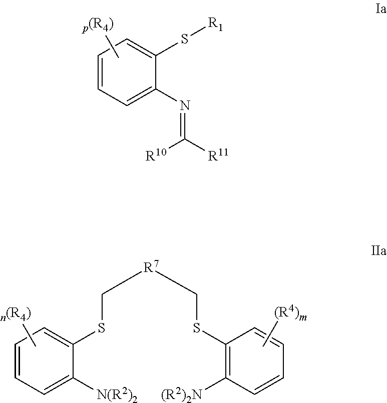
wherein,
R¹ is C₁₋₆ alkyl, C₂₋₆ alkenyl, aryl, heteroaryl, C₃₋₁₀ cycloaliphatic, or 5-10 membered heterocycloaliphatic having 1-3 heteroatoms independently selected from N, O, or S, any of which is optionally substituted;
R² is independently hydrogen, or each pair of R² groups is —N═CR¹⁰R¹¹, wherein one of R¹⁰ and R¹¹ is hydrogen and the other is optionally substituted phenyl or an optionally substituted 5-6 membered heteroaryl having 1-2 heteroatoms independently selected from N, O, or S;
R⁴ is —Z^{B}R⁶, wherein each Z^{B} is independently a bond or an optionally substituted branched or straight C₁₋₆ aliphatic chain wherein up to two carbon units of Z^{B} are optionally and independently replaced by —CO—, —CS—, —CONR^{B}—, —CO₂—, —OCO—, —NR^{B}CO₂—, —O—, —NR^{B}CONR^{B}—, —OCONR^{B}—, —NR^{B}NR^{B}—, —NR^{B}CO—, —S—, —SO—, —SO₂—, —NR^{B}—, —SO₂NR^{B}—, —NR^{B}SO₂—, or —NR^{B}SO₂NR^{B}—,
R⁶ is independently R^{B}, halo, —OH, —NH₂, —NO₂, —CN, —CF₃, or —OCF₃,
R^{B} is independently hydrogen, an optionally substituted aliphatic, an optionally substituted cycloaliphatic, an optionally substituted heterocycloaliphatic, an optionally substituted aryl, or an optionally substituted heteroaryl; or
two R⁴ groups together with the carbon atoms to which they are attached form an optionally substituted 5-6 membered ring having 0-3 heteroatoms independently selected from N, O, or S;
One of R¹⁰ and R¹¹ is hydrogen and the other is optionally substituted phenyl or an optionally substituted 5-6 membered heteroaryl having 1-2 heteroatoms independently selected from N, O, and S; and
R⁷ is an optionally substituted phenyl or an optionally substituted 6 membered heteroaryl having 1-2 heteroatoms independently selected from N, O, or S and
Each of m, n, and p is independently 0 or a positive integer from 1-3, and a pharmaceutically acceptable excipient,
wherein administration of said pharmaceutical composition to said subject provides for a minimum period of time of 6-12 hours a concentration in a target tissue of at least about 2-10 times the MIC of the bacteria.

21. The pharmaceutical composition of claim 20, wherein the compound of Formula Ia or Formula IIa, or a pharmaceutically acceptable salt thereof, is selected from:

| Compound Name | Structure |
|---|---|
| 2,2′-((1,2- phenylenebis(methylene))bis(sulfanediyl))dianiline | 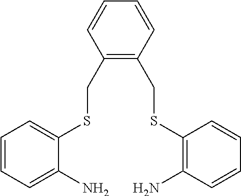 [SMILES: Nc1ccccc1SCc1ccccc1CSc1ccccc1N] |
| 2,2′-((1,4- phenylenebis(methylene))bis(sulfanediyl))dianiline | 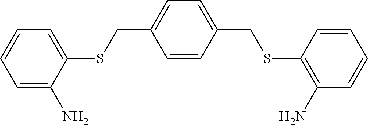 [SMILES: Nc1ccccc1SCc1ccc(CSc2ccccc2N)cc1] |
| (NZ,N′Z)-2,2′-((1,2- phenylenebis(methylene))bis(sulfanediyl))bis(N- (pyridin-2-ylmethylene)aniline) | 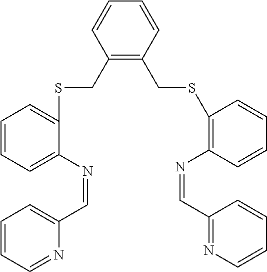 [SMILES: C(=N/c1ccccc1SCc1ccccc1CSc1ccccc1/N=C\c1ccccn1)/c1ccccn1] |
| (NZ,N′Z)-2,2′-((1,2- phenylenebis(methylene))bis(sulfanediyl))bis(N- (thiophen-2-ylmethylene)aniline) | 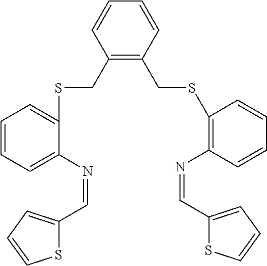 [SMILES: C(=N/c1ccccc1SCc1ccccc1CSc1ccccc1/N=C\c1cccs1)/c1cccs1] |
| (Z)-2-(benzylthio)-N-(pyridin-2-ylmethylene)aniline | 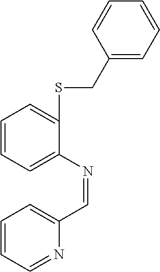 [SMILES: C(=N/c1ccccc1SCc1ccccc1)/c1ccccn1] |
| 2,2′-((1,4- phenylenebis(methylene))bis(sulfanediyl))dianiline | 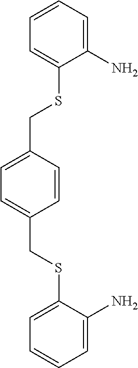 [SMILES: Nc1ccccc1SCc1ccc(CSc2ccccc2N)cc1] |
| 2,2′-((1,2- phenylenebis(methylene))bis(sulfanediyl))dianiline | 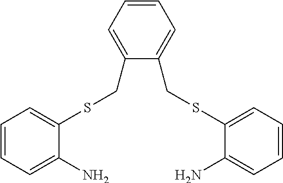 [SMILES: Nc1ccccc1SCc1ccccc1CSc1ccccc1N] |
| (E)-2-(benzylthio)-N-(pyridin-2- ylmethylene)aniline | 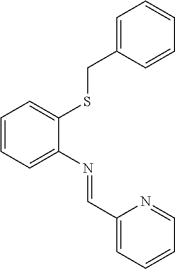 [SMILES: C(=N\c1ccccc1SCc1ccccc1)/c1ccccn1] |
| (NE,N′E)-2,2′-((1,2- phenylenebis(methylene))bis(sulfanediyl))bis(N- (pyridin-2-ylmethylene)aniline) | 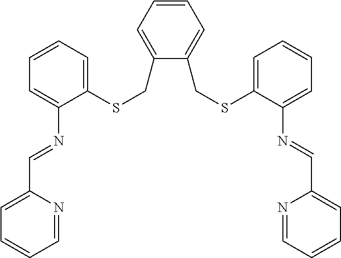 [SMILES: C(=N\c1ccccc1SCc1ccccc1CSc1ccccc1/N=C/c1ccccn1)/c1ccccn1] |
| 2-(methylthio)-N-(2-thienylmethylene)- benzenamine copper (II) bromide | 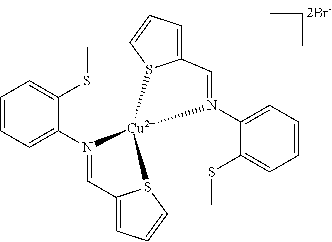 |
| 2-(methylthio)-N-(2-pyridylmethylene)- benzenamine copper (I) bromide | 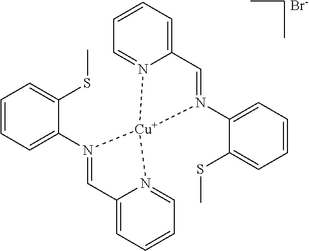 |
| 2-(methylthio)-N-(2-pyridylmethylene)- benzenamine copper (I) iodide | 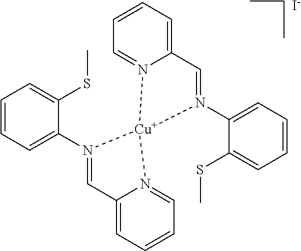 |
| 2-(methylthio)-N-(2-pyridylmethylene)- benzenamine copper (II) bromide | 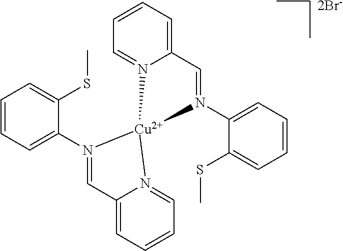 |
| 2-(methylthio)-N-(2-phenylmethylene)- benzenamine copper (II) bromide | 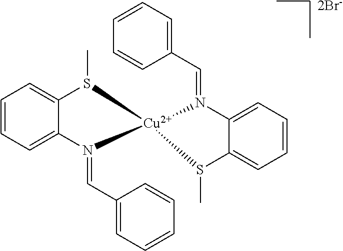 |

22. A method for sterilizing a solid surface, the method comprising: contacting the surface, with a therapeutically effective amount of a compound of Formula Ia, a compound of Formula IIa, or one or two compounds independently selected from Formula Ia or Formula IIa complexed with a metal core, or a pharmaceutically acceptable salt, prodrug or hydrate thereof, or an antimicrobial composition comprising a therapeutically effective amount of a compound of Formula Ia, a compound of Formula IIa, a compound of Formula Ia or a compound of Formula IIa complexed with a metal core, or a pharmaceutically acceptable salt, prodrug or hydrate thereof, and a solvent or diluent 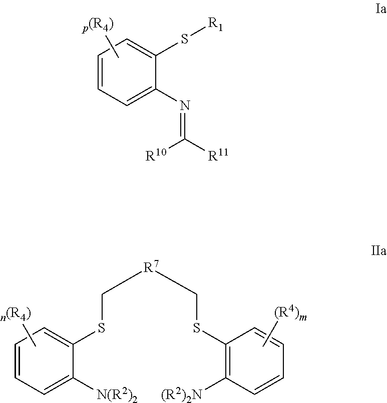
wherein,
R¹ is C₁₋₆ alkyl, C₂₋₆ alkenyl, aryl, heteroaryl, C₃₋₁₀ cycloaliphatic, or 5-10 membered heterocycloaliphatic having 1-3 heteroatoms independently selected from N, O, or S, any of which is optionally substituted;
R² is independently hydrogen, or each pair of R² groups is —N═CR¹⁰R¹¹, wherein one of R¹⁰ and R¹¹ is hydrogen and the other is optionally substituted phenyl or an optionally substituted 5-6 membered heteroaryl having 1-2 heteroatoms independently selected from N, O, or S;
R⁴ is —Z^{B}R⁶, wherein each Z^{B} is independently a bond or an optionally substituted branched or straight C₁₋₆ aliphatic chain wherein up to two carbon units of Z^{B} are optionally and independently replaced by —CO—, —CS—, —CONR^{B}—, —CO₂—, —OCO—, —NR^{B}CO₂—, —O—, —NR^{B}CONR^{B}—, —OCONR^{B}—, —NR^{B}NR^{B}—, —NR^{B}CO—, —S—, —SO—, —SO₂—, —NR^{B}—, —SO₂NR^{B}—, —NR^{B}SO₂—, or —NR^{B}SO₂NR^{B}—,
R⁶ is independently R^{B}, halo, —OH, —NH₂, —NO₂, —CN, —CF₃, or —OCF₃,
R^{B} is independently hydrogen, an optionally substituted aliphatic, an optionally substituted cycloaliphatic, an optionally substituted heterocycloaliphatic, an optionally substituted aryl, or an optionally substituted heteroaryl; or
two R⁴ groups together with the carbon atoms to which they are attached form an optionally substituted 5-6 membered ring having 0-3 heteroatoms independently selected from N, O, or S;
One of R¹⁰ and R¹¹ is hydrogen and the other is optionally substituted phenyl or an optionally substituted 5-6 membered heteroaryl having 1-2 heteroatoms independently selected from N, O, and S; and
R⁷ is an optionally substituted phenyl or an optionally substituted 6 membered heteroaryl having 1-2 heteroatoms independently selected from N, O, or S and
Each of m, n, and p is independently 0 or a positive integer from 1-3.

23. The method for sterilizing a solid surface of claim 22, wherein the compound of Formula Ia or Formula IIa, or a pharmaceutically acceptable salt thereof, is selected from:

| Compound Name | Structure |
|---|---|
| 2,2′-((1,2- phenylenebis(methylene))bis(sulfanediyl))dianiline | 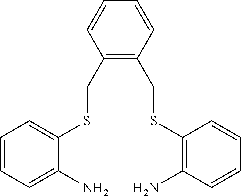 [SMILES: Nc1ccccc1SCc1ccccc1CSc1ccccc1N] |
| 2,2′-((1,4- phenylenebis(methylene))bis(sulfanediyl))dianiline | 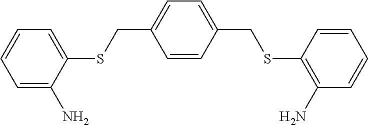 [SMILES: Nc1ccccc1SCc1ccc(CSc2ccccc2N)cc1] |
| (NZ,N′Z)-2,2′-((1,2- phenylenebis(methylene))bis(sulfanediyl))bis(N- (pyridin-2-ylmethylene)aniline) | 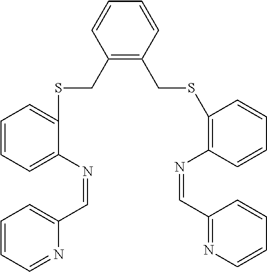 [SMILES: C(=N/c1ccccc1SCc1ccccc1CSc1ccccc1/N=C\c1ccccn1)/c1ccccn1] |
| (NZ,N′Z)-2,2′-((1,2- phenylenebis(methylene))bis(sulfanediyl))bis(N- (thiophen-2-ylmethylene)aniline) | 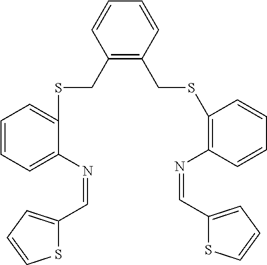 [SMILES: C(=N/c1ccccc1SCc1ccccc1CSc1ccccc1/N=C\c1cccs1)/c1cccs1] |
| (Z)-2-(benzylthio)-N-(pyridin-2-ylmethylene)aniline | 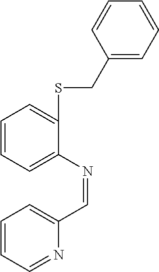 [SMILES: C(=N/c1ccccc1SCc1ccccc1)/c1ccccn1] |
| 2,2′-((1,4- phenylenebis(methylene))bis(sulfanediyl))dianiline | 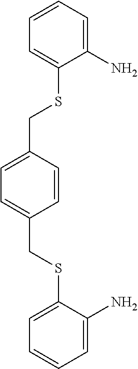 [SMILES: Nc1ccccc1SCc1ccc(CSc2ccccc2N)cc1] |
| 2,2′-((1,2- phenylenebis(methylene))bis(sulfanediyl))dianiline | 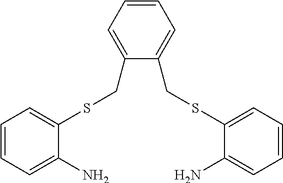 [SMILES: Nc1ccccc1SCc1ccccc1CSc1ccccc1N] |
| (E)-2-(benzylthio)-N-(pyridin-2- ylmethylene)aniline | 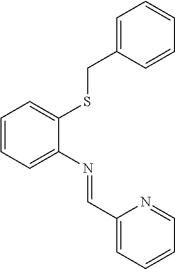 [SMILES: C(=N\c1ccccc1SCc1ccccc1)/c1ccccn1] |
| (NE,N′E)-2,2′-((1,2- phenylenebis(methylene))bis(sulfanediyl))bis(N- (pyridin-2-ylmethylene)aniline) | 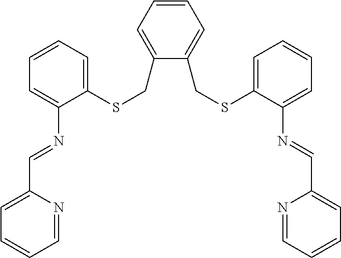 [SMILES: C(=N\c1ccccc1SCc1ccccc1CSc1ccccc1/N=C/c1ccccn1)/c1ccccn1] |
| 2-(methylthio)-N-(2-thienylmethylene)- benzenamine copper (II) bromide | 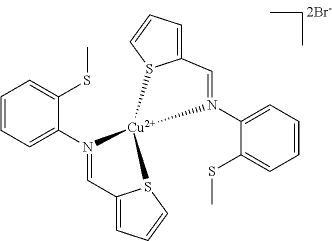 |
| 2-(methylthio)-N-(2-pyridylmethylene)- benzenamine copper (I) bromide | 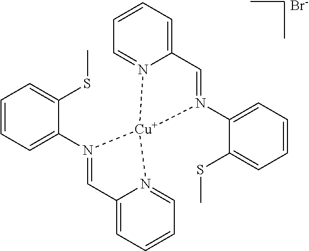 |
| 2-(methylthio)-N-(2-pyridylmethylene)- benzenamine copper (I) iodide | 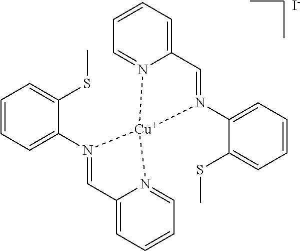 |
| 2-(methylthio)-N-(2-pyridylmethylene)- benzenamine copper (II) bromide | 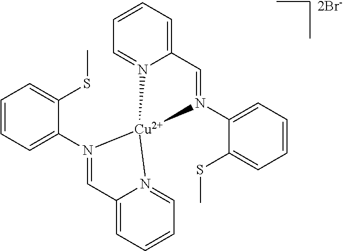 |
| 2-(methylthio)-N-(2-phenylmethylene)- benzenamine copper (II) bromide | 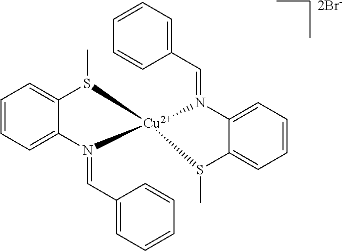 |

24. A medical device or a portion thereof, coated with or having adsorbed thereto, an antimicrobial composition, the antimicrobial composition comprising a compound of Formula Ia, a compound of Formula IIa, a compound of Formula Ia or a compound of Formula IIa complexed with a metal core, or a pharmaceutically acceptable salt, prodrug or hydrate thereof, or an antimicrobial composition comprising a therapeutically effective amount of a compound of Formula Ia, a compound of Formula IIa, a compound of Formula Ia or a compound of Formula IIa complexed with a metal core, or a pharmaceutically acceptable salt, prodrug or hydrate thereof, and a solvent or diluent.
